SEQUENCE LISTING (289 sequences)

SEQ ID NO: 1 - DNA, 177 bp, rat
  misc_feature (1)..(177): Clone #2  (MLSSH)
accaggagctgatgaacgtgaagctggcgcttgacatcgagatcgccacctaccgcaagctgctggagggcgaggagagcaggctggagtctgggatgcagaacatgagcatccacacgagaccaccagtggctacgcaggaggactgagttcatcctacgggggactcactagccc

SEQ ID NO: 2 - DNA, 533 bp, rat
  misc_feature (1)..(533): Clone #4 (MLSSH)
ataggcttgtaagtgcccgaagtgtaagcccaactacagagatggtctcaaatgagtctgtagactacagagctacttttccagaagaccagtttcccaactcctctcagaatggagcctgcagacaagtgcagtatcctctgacagatctgtcccccatcctgacgagtggagactctgatatatccagcccattactacaaaacactgttcacattgacctcagcgctctaaatccagagctggtccaagcggtgcagcacgtagtdtttggacccagtagcctgattgtgcatttcaatgaagtcataggaagagggcattttggctgtgtctatcatgggactttgttggacagtgacggaaagaaaattcactgtgctgtgaaatccttgaacagaatcacagatatagaagaagtctcccagtttctgactgagggaatcatcatgaaagatttcagccaccccaatgttctctcactcttgggaatctgcctgcggagttaagggtcccctctggtgtttctgccc

SEQ ID NO: 3 - DNA, 613 bp, rat
  misc_feature (1)..(613): Clone #5 (MLSSH)
acagagatatatttgtgcgacagaacattaggagtcttcccacagatgacattgcagctctaccgattggaagaacagttgcggaaactccaggaagagagaacgtgtaaagtgtgtatggaccgagaggggtccctcggtggttcattccctggtggccattctggtcgttgtgcaaaggaattgtggcccccttcttctgagggaagtggttccatctggcagagggactatccaaagggcacagtgcggcacaattttctctcctgaacaggacttggaactgatggtccaccggtttgtgaaccccaggccacggaaggaggcagaaggttcaatggttcactcccaggctcattaggaaactttgaggccgggccttggattgaacatgagagcaactggcctctaaaatagtgtaaaaaatggaaaaaacaattgggaaaaaaaaaaacctttttgttctaaaactcaaagaaatggatttactttatcataataatttttaatttggttttcctttaaaaagttggctattttattttcccaactcagaagaaactggtttttcagtaaaaggtattttttcatactattctgtcattctaaaagc

SEQ ID NO: 4 - DNA, 335 bp, rat
  misc_feature (1)..(335): Clone #6 (MLSSH)
acagatggcattgttcatgacgcactccaccaccatcttcccgtccttcagttttctcgttatcgtgctttctttcccttcccacttctggtgctggaccagggcaccgtctgttaaggtgcagaccgtctcatttttcctgccatcagctgtggtttcatcaaacttctctcccaaggtgcaagaaaacacggtcktcttcaccgtgctctcagttttkacggtgaggttgttgccgtctagggtaatgatgcagtctggtttggccattkgcacccatctccctaagagccagccctactcctagttccttcatgtagttctcaaacccgtgg

SEQ ID NO: 5 - DNA, 582 bp, rat
  misc_feature (1)..(582): Clone #7 (MLSSH)
actaatcattttcaggggacagagaaggcttgctgctttcctcatgctgcccctgagagctgactacccagtcttccacaggcacctgagtgagcaggggagggagggaaatcctggctcctggacttcctccacactgaaactgtgggcgtcttctgcatgtataacatgcgtgtctaaggacagcaagagagccctgccgtctcggtgagctctagctgcctccgcaggaggggcagtgcgccttcattttatggttagaaaggcacaaaatttttatcaacctgaaacattcctttttctttttgcctcaattatcatggagttttccaattctcttctggtttttgtttaacaaacgctttaacaatgtaatatatttatttttccccctattctggaggattcgtctgaaggattgttattgaaacaggatgcgtgggactctctgaatcatcttcatagtgagtctttatgactgtaagattgtaaatacagattatttattaaccctgttctacctggaattaaactgcatagagcaaatgtttgcaagcaggtagctgaggctgtcagcaaatc

SEQ ID NO: 6 - DNA, 289 bp, rat
  misc_feature (1)..(289): Clone #8 (MLSSH)
acaataagcttcccaggagacgtgtcaacatttttggtgaagaacttgaaagacttcttaaacagaaatatgaagggcattggtatcctgagaagccatacaaaggctcagggtttagatgcatacacgtaggggagaaggtggacccggtgatcgagcaagcgtccaaagagagtggtctggacattgacgatgttcgtggcaatctgccggcaggatctgagcgtgtggatcgacccgtttsaggtttcctaccaaatcggtgagaagggaccagtgaaggtgctgt

SEQ ID NO: 7 - DNA, 574 bp, rat
  misc_feature (1)..(574): Clone #9 (MLSSH)
cggccgccagtgtgatggatatctgcagaattcggcttagcgtggtgcggccgaggtactattttacctatgacccatggattggcaagttactgtatcttgaacacttctttgtgatgagtgattaccgaggctttggtataggatcacaaattttcaagaatctaagccaggttgctatgaagtgtcgctgcagcagtatgcacttcttggtagcacagtggaatgaaccatctatcaacttctacaaaagaagaggtgcttcgcatctgtccagtgaagagggatggagactattcaagattgacaaagaatacttgctaaaaatggcagcagaggagtgaggtgtgccggtatagacaatgaccacctccattgtgctttagaataaattctcaacttcccttgctttctatctcgtttgtagtgaaataatagagtgagcatccattccaaagctttattaccagtggcgttgttgcatgtttgaaattcggtctgtttaaaatggcagtcatgtatgtggtttgcaggcagaatcctgaacatcttttgatgaagaacaaggtggtat

SEQ ID NO: 8 - DNA, 565 bp, rat
  misc_feature (1)..(565): Clone #10 (MLSSH)
actctcagaaagaagacaagtatgaagaggagatcaaggttctctctgacaagctgaaggaggctgagacccgggctgagtttgcagagagatcagtaaccaaattggagaaaagcattgatgacttagaaggtaagatctttaatattttaaattcagtcctatggttgactataactctcttgataaagcagtcgtccaatccgagggcttccaccgggacacgcctgtctgttgtcacacttcctgtctgactttcctctggggttctcacctgtcaggatgaaactcttgaacccgagtcctcagttcttggggcgacttagctgccagacagcttccgtctaatttcttgcaattcagaagtgggtctgggctgcttttcactgagcgaagtaattatagaagaatgtgactctggttttctgtatctctggcaatatctcctaattgtcagaacagtggagatttgttttagacacagcccatcttccaagtgtgacccctgaagaagggcttgatttcagtcattcattggcaaaatattctatattttgaagggttg

SEQ ID NO: 9 - DNA, 381 bp, rat
  misc_feature (1)..(381): Clone #11 (MLSSH)
accaactccttccatatcccacttgactggactggctccgcccccatcctccagcatccgcagttccaactgcactggtttgcagcttcgggaaacagtctactgttgtaaatactccgctggggtggccctactttttggatgcggccacagcaattccccctgacggtgaccaggacagaaggaagagaaatctacaggctgagagaggcccggagagtccacggcaaacctcctcttcccgttttcctgaccaggctagaagattacccggcaggtggacaatagattcatcctctgagcgctgcgctttgcccggcatctgtgcaggaatctggtcgcctgacttccttatgagactgtgaacaactccacgacatg

SEQ ID NO: 10 - DNA, 599 bp, rat
  misc_feature (1)..(599): Clone #12 (MLSSH)
actaccttataaggaagcacttagtggactccttcaagttccatttgctaatgattaacacactgtctgggctggccagtttctcatgcatgcagcttggtgattgagcacagtcaggcctttgtattaaaactgaaaaatggaaaaaacaaattcaaactctcagatggcttctagttcaatctgttcagataaatgtcagcagccagtttaccgcaaacaaacatttaaaattggtttacctcaggatgatgtgcaggaaacgggtgaaggctaaactttgagacacagccctagatgtaggacagtcctcttcaagtgccccaatcccatggtgacagtgacaccctggtggcgagcccgagtgtgatggttcaccgctgtgcacattgcattggagtgaagtgaacaaggctgtcaccaaatcacacacgttttaataagaaatgtttactaagggagcatctttggactctctgttttaaaccttgtgaaccatgactcggagccagcagagtaggctgtgtctgtggacttgagcacaccatcaacattgctgttcaggaaattataatttacgtccattccaagttgtaaat

SEQ ID NO: 11 - DNA, 569 bp, rat
  misc_feature (1)..(569): Clone #13 (MLSSH)
gccgcccgggcaggtacaatgtaatgtttatttcaattgtttatatggatagtctgagcaggtgcggttctgattctgattgctatcagtaatgccccaaactttcttgtgaggatctgaaacccaaaggtggcagttatgagattgggacatggatcttgcccaatcagaagctgtttttttttaaaaaaaaaacctggagagtcaggagcaccataagatatgatggtctctatgtgccaacgggttccccgaagcgggtcacaaacacatacggcaacaccacatgtgaatgatacttttaaactgacaatttcgtcttctctgaaaaaagacatacacagagtttgtataacactttacttaacgacacttgctagaaaattacattctgtttttaaaaaaaataaggaaaatgtaaagaagaatatctaatatgtcagagaaagttggaaacaaatcacagctcgcatccccatagtcgcagatcacgatgtaaatcactgtaattaaaattggtgttaaatcctttgggttatccactgccttaaaatgacacttatttcctt

SEQ ID NO: 12 - DNA, 399 bp, rat
  misc_feature (1)..(399): Clone #14 (MLSSH)
accttcctcaggactagggctcatgtctcctgtcctcagaggaagagaaacaatgtgagatccctttgtttaaactgtgaatgtatcctaaagcatagtcacaccagcacggggtctaactaactttagaacccattaatacaggcatagaattgggcctttgtttgggagctttgggggaagggaggcccatggaggcttctggagtttcatgggaggcctgcgggggacttcaaatggcggcatttaaggtgggaatgtttgcccgggatatgctggtagctgaactctgataactaagcacaggttgcctcatcctctccctcatctttgcttaacagccaggcaatgcattagactggtctttgggacttgccctggagcaatacctaatgaaca

SEQ ID NO: 13 - DNA, 649 bp, rat
  misc_feature (1)..(649): Clone #15 (MLSSH)
acagcgttctgactacttgaatacatttgagtttatggacaaacttggagaaaacttgaaggccaaactagctcaggccaaactttaaggtcaaacctgggcttagggcaaatctttgtggtaactaagtccacagtttacatatttcctttttgttttaataacactcaaggttaaaaacaagaatcattttgtaatttgtttagaagacgaagttgaacttttctatatgtttacagcctattttctttttcatacagttattgccaccttaatgaatgtggtggggaaggtttttttaattgtattttactgtgtaatagcagtctaggaattgtgttagtatctgttcacaattaactgtcactttttctttgctctaatgtaaatgaccaaaatcagaaatgctccaagggtgaacaatcactacactatgtttccccgtaaaggtgaaggagaggctcacctcccctactgtccatgagtgtgaacacagggagcttttgtgtgcaaatgttgcactgtgtgtggaagagactggaacaaatagaatgtgtgttgctgaaatgcatggcagccatgtttgtagagagcacttatgtgttcaatatactaattatacgttaagaccactggagaattccaagtc

SEQ ID NO: 14 - DNA, 554 bp, rat
  misc_feature (1)..(554): Clone #16 (MLSSH)
actgggaaagttggttatcttgggggttcagcttcattactacttagggtttccctgcccactctgcaggagcagaatgttggacaggtagccagtgggatgccactgcttgccgccactgtccctgggcttagtttaaggggacgtgtatacctaatccacacacgagttagaagtatgagttggctggtcaacttgaacattgttacaggcgggtgggtgttagtggggggttattttttggtgggactagcatgtcactaaagcgggccttttgatatattaaattttttaaagcaaaacaagtttagattttaatcaagttcgtagggtttctaactttacagaattgcctgtttgtttcaatgactccttccacttggctcttaggggaactgaggacaggcctggagttaatacacttgtcattctgtgtcctagtgtcctcttcctccggcagactgtccccttccttctgaaaaagccgatagagtcttgttttatttttcttttataataaacacaccccacctccaaaaaaaaaaaaaaaaaaa

SEQ ID NO: 15 - DNA, 395 bp, rat
  misc_feature (1)..(395): Clone #17 (MLSSH)
acatagatcaracgcttttgccagtgatcatcctagaattgctgagtcctaatgctaaaacagcaggcagacaaagacagtgagaagttggaggaggcttgctcccccttcaatattccaatccagactacagcgggacatggctgaacaaataggacacggattccccattgtgggttcttcgaatggcttcagccagaatcattgaaatgtcgatcacctgaattttggaacagtgtttcattttgtcctcttgtggaatggtattggtgacaacaactgcttcaaaggcagcattatttattctggaaatagctggcccagagaagattccatgggtagaatagcataaactttgtggcaccagctgagagtaacttatctgcagcatgaca

SEQ ID NO: 16 - DNA, 384 bp, rat
  misc_feature (1)..(384): Clone #18 (MLSSH)
ggtcacacacggccatttatgtaaagtcctattttcttttttaaaaaagttggaatttgtttttccttcttagaaggaaacaatggtatttaacaagtttgcaaagatgagtttgttacctcgtatatgtaaaatggacagctgactacaagacaccatgtaggaaaccactagttaatgtattttattttatatgcaaattacttcaagagttagatcaggctttgtggaaagctgtagtgaaataccttaagctgttaagtgtaagtgcggagtaggaactgctcagtcgattggttgatttagacttcttatgtctgcatttgttactgtgctaataaaccatattaagatcaaaaaaaaaaaaaaaaaaaaaaagcttgt

SEQ ID NO: 17 - DNA, 114 bp, rat
  misc_feature (1)..(114): Clone #19 (MLSSH)
catgctcgagcggccgccagtgtgatggatatctgcagaattccagcacactggcggccgttactagtggatccgagctcggtaccaagcttggcgtaatcatggtcatagctg

SEQ ID NO: 18 - DNA, 553 bp, rat
  misc_feature (1)..(559): Clone #20 (MLSSH)
aagcattcgggaagaggcaaggggagtgttcattcacacacacagtagcttcgggtctgtgaggtctccttgttcccgggtaggtttctaaagacggagaaaaaaatgattctggttatcaagactactgtgaccgtattagatcccagaatgggcaagcatcagtgtgtgaccatgcgaacaaaaggaatttttttttttttttgaggaaatgtctgtgttttagaaggtatgtgtgtatcaaggaaattggggaaaaaaaaaacttactgtagaaccaaaccccctctcaggcacaggaccaatgtcaaactatctaaaaatatgaactgaaccaaagtattccaaacgtggttattccgatccatggttttgtttttctctttaaaaaaaaaaaaagtatttacagaaattgtataaaagactttgtgaattctgtgagagcttccagtcctcacatcccaaatgctggggttgcatatttgcctgtagaatttcaagactcataattttggccttgctaaagaccacgtttttaaatcatgaaaagccc

SEQ ID NO: 19 - DNA, 512 bp, rat
  misc_feature (1)..(512): Clone #21 (MLSSH)
acttggagcacgatattcttctgtctcacagccatagccagctccttggcgtgtcccatgcttaggccagatacctcatcacacgtgaggccaagaaagaggcctgtgggaagactggcaaagtcaacggtgccggtcacagcatggagaagtgcggcctcctggcttgtccttgtagtctgggtctgcagaagtggcaaccacccaagactaaggagaagcagtagaaagctgcggctgcagatggggcttccacaggaccccagcagggtttgggctgttggcaaggccatggtctgtggataacaatggttctgaggtccccacctgtcccacccctcaactcacaccactgtttggacacagtgaggggcctgttggcagtagtgtctaccgggcagatggaccttgtgaatgaaattccctttacctacgagcagctcagcatcttcaagcacaaactggacaagacctacccacaaggctatcccgagtcccagatcaagcagctg

SEQ ID NO: 20 - DNA, 556 bp, rat
  misc_feature (1)..(556): Clone #22 (MLSSH)
tgctggaattcggctttctgagcggccgccgggcaggtaccaggacgctcagccacaatcttaccaaaggcttcagcactcttggtccaggcattgagatcccactgaaacttcatctcaccatgaggctccacaggatccacaatcattttgagggcccgatgcagctggaaataaatcagcttcttagagagcaagagggctgtattgttatcactctccaaagcccaagaggcaaagcgcaaaatgtgttcctggtgcttctggattttggtcatggtccagtgctgacgctccagacgttcctgctgaccctcagtcaccttctgggcatcattgaccagaactcgtccccgtttattcagctccttcatgatctgcagaatggccatcttcacatcaacctgcactcgcttctgcacatcagacacctggcggatcgagcttcgaacctccttggtgtttttctgcagtgtggcatgtttgtccccaagacgtttcaccagtgaagccaagagtttacgttggttccttactgcatcttccaagaattggt

SEQ ID NO: 21 - DNA, 530 bp, rat
  misc_feature (1)..(530): Clone #23 (MLSSH)
gcaggtacaagctttttttttttttttttttttttttgagaagacataatggttatgaaattggcttgaaaccagttgtagggggttcgaatccttcctttcttatttaacttttacataggaaggttcttcgaatgtgtggtaaggtggggggcatccatgcagtcattctaggttagttgaagagtaggaaattgagagcacttctcgttttgatgcgaaggcttctcaaatcatgaagatcattacaaggacggccgtaagtgagatgaatgagcctatagaggagactgtatttcatgtggtataagcatctggataatcagagtaacgacgaggtatccctgctaatcctaggaagtgttgaggaaaaaatgttatgtttacacctacaaatataatggcaaagtgggcttttgctctgtgtcatttagggtatagcctgagaatagtgggaatcagtggacgaagccagctatgatggcgaatactgctcctatagataagacatagtggaatgagctactacg

SEQ ID NO: 22 - DNA, 869 bp, rat
  misc_feature (1)..(869): Clone #24 (MLSSH)
caacttgactcctcccactaataatgctcgctagccaaaatcacataaaaaaagaaaatataatgcatcaaaaactttacatctcaatacttattagcctccaaattttactcatcataacattctccgcaacagaactaattttattttatatcctgttcgaagccactctaatcccaacactaattatcattacacgatgaggcaaccaaacagaacgcttaaatgcaggaatttatttcctgttttatacactaattggctccatcccactcttaattgccctcatttcaatccaaaactcaataggaacactcaacttcctaatcctttccctcacaacacaccccctaccctcaacatgatccaacaccattctatgactagcatgtataatagcatttataatcaaaataccattatacggagtccatctatgattaccaaaagcccacgtagaagctccaattgcaggctctataattttagcagcaattctcctaaagctagggggttatgggataatacgagtttccatcattctagaccccctaacaaaatctttagcctacccattcatcatcctctcattatgaggcataattataactagctcaatctgcctacgccaaacagatctaaaatcattaattgcttactcatcagtaagccatatagccctagtcatcacagccattataatccagacaccatgaagcttcatgggagctacaatactaataatcgcccacggcttaacctcatcactcttattctgcctagcaaacaccaactacgaaggaattcacagccgaactataattatagctcgaggattacaaataatctttccattgatagcaacatga

SEQ ID NO: 23 - DNA, 435 bp, rat
  misc_feature (1)..(435): Clone #25 (MLSSH)
acttcctcattgagcgtggaaagaacatgtgtggcctggctgcctgtgcctcctaccccatccctcaggtataagccacggctgcacaggcaactgcttggcagacaaagggaggaactggtcctacgatgagaatgccgccctggagaaagttgttcagaaatccacccagaggccctctcactcctgagtctagacgcctaaagacagtaaggaagaacttgaccagcaacaagcccgcccgtgtgacgacatcaccagccatacgctttgtttgaatatggtttttaaatgacccaaaaccatgtggacctagaatcttctcttttcagctctcttcatatagggaaagctccgatggctaccttttctatgttgtatattcaataaatgcagtgaacgcctgcctgggctgggcctagaaaaaaaaaaaaa

SEQ ID NO: 24 - DNA, 689 bp, rat
  misc_feature (1)..(689): Clone #26 (MLSSH)
actttggtggttttagtgaggttgaatccatagagctgcctatggacaacaagaccaataagagggcggtggggttctgtttcattacctttaagggaagagggaaccagtgaagaagataatggagaagaaataccacaatgttggtcttagtaaatgtgaaataaaagtagccatgtcgaaggagcagtatcagcagcagcagcagtggggatctagaggagggtttgcaggaagagctcgcggaakaggcggtgatcagcagagtggttatgggaaagtatccagacgaggtggtcatcaaaatagctacaaaccatactaaattattccatttgcaacttatccccaacaggtggtgaagcagtattttccaatttgaagattcatttgaaggtggctcctgccacctgctaatagcagttcaaactaaattttttctatcaagttcccgaatggaagtatgacgttgggtccctctgaagtttaattctgagttctcattaaaagaatttgctttcattgttttatttcttaattgctatgcttcagtatcaatttgtgttttatgcccccctcccccccagtattgtagagcaagtcttgtgttacaagcccagtgtgacagtgtcatgatgtagtagtgtcttactggttttttaataaatccytttgtataaaaaaaaaaa

SEQ ID NO: 25 - DNA, 691 bp, rat
  misc_feature (1)..(691): Clone #27 (MLSSH)
cttctcacaagagctgaacaataaaggcggtgactgcagaakaatcctggtgttccaagtcgttcttgccggckagaccaccgtgcgggacagaaaatgagcatcacggacatcctgagcgctgaggacattgcggcagccctgcaggaatgccaagacccagacacctttgaaccacaaaagttcttccagacatcgggcctctccaagatgtctgccagccaagtgaagggatatcttccggttcatagacaacgaccagagtggatacctggatggagatgagctcaagtatttcctacagaagttccagagtgacgccagagaactgactgagtcagagacaaagtccttgatggacgcggcgbacaacgacggagacgggaagattggggcggatgaattccaggaaatggtgcactcttaaggcccaagtctacggaagagagagaggggaaggggcaaatctgcctgaaggtttcaaaagccctgggactgggaagctcccaacctctcccgaactactttaccgagtcccccgaagcccttctgcaccatgttccttgttgagggaatgcaatggtttgtgatggtgggtaggtcctgataacctctgtaagcacccccccctctggcagacaatgcctttaaaaaccacccaataaaatcatttctcatcaaaaaaaaaaaa

SEQ ID NO: 26 - DNA, 390 bp, rat
  misc_feature (1)..(390): Clone #28 (MLSSH)
acctcttgagccttgaactttgacctctgcagcaataaagcagcatttctacacatataaggtcatttttttaagaaaaagaatgcacagagttgttacatttttaagtgctgcatttaaaagatacagttactctagtttgattaaattcttgcaaagtatcccttctgtaaaatttgtgatacaatgctgtgccctaaagtgtatttttttactaatagtttattatggcacatcagcacgatttctgtttagataatacaccactacattctgttaataatcattaggtgtgaaaggatttcttttgtggttattaaaaatctcaaatttctaaatctgcaagaataaaactttttaaaaaaaaaaaaaaaaaaaaaaaaagcttgt

SEQ ID NO: 27 - DNA, 603 bp, rat
  misc_feature (1)..(603): Clone #29 (MLSSH)
acatcttcttttcctccacgaaacaggatcaaataaccccacaggattaaactccgacgcagacaaaatcccattccatccatattatacaatttaaagacctccttaggttgttattttattatttacttatttatttccttaattaacccttagttacttattctttcccaggaccttacttagggaggacccaggacaatttattacattccggctaacccccttcaacaccccaccccacatcaaaccagaatgatatttttctctttgcctacgctattctacgctccattcccaacaaactaggaggggtcgtagccctaatcttatcaatcctaatcttagccttcctaccattcctgcatacctcaaaacaacgcagcttaacattccgcccaatcacccaaatcctttactgaatcctagtagccaacctcctagtcttaacatgaatcggaggccaaccagtagaacacccatttatcattattggtcaactagcctccatcagctatttttcaattatcctcattctcataccaatctctggaattgttgaagacaaaatgttaaaatgaaattaacgaaaaaaaaaaaaaa

SEQ ID NO: 28 - DNA, 354 bp, rat
  misc_feature (1)..(354): Clone #30 (MLSSH)
accaaccaccccaagtcctaaactttctccaggggttgactcaagactaaagggaacctctggttgcccacccgtctctgcacaccgcacatccctccatgttccattcctggaaggagaggcccacgtccgcttgctgccccttgggtcaccagatcctgcctcttatctcctgagacccctcttgaccctcgctctggagcctcggttgacaagaggactgtcgggtctggagagatagatgggctctgggtggttggcgagctggctatgggacctctgctggcttgctacccaagttaacaagcagattccaaaatacatttgtgctctccactggaaaaaaaaaaaaaa

SEQ ID NO: 29 - DNA, 624 bp, rat
  misc_feature (1)..(624): Clone #31 (MLSSH)
acaagctttttttttttttttttttttttttattatgaatgacattttattcagtcattttctttacaactgaaactctgggaattcaaagttaacatccttgcctgtgagcttcttgtacacgccagaaaaagtttcgaccttatgctccacgttgttctgctgtgctttgtctaaatgaacttttatgagccggctgccatccagtttcacacggatcctcttgcccacaatttcacttgggaagaccaaatcctcaaggatggcgtcgtgcactgctgtcagggtgcggcttctggggcgcttttgcttatttttcgtacggctttttcgggttggcttgggcagaatcctcctctgagcaatgaagactacgtgtttcccactgaactttttctccaattcacgaactagccggacttggattttctggaaagatttcagctgaggaactggtacaaaaattatgatggcttttcgaccaccaccgacttcgatttcctttgtcgcggtgatgttgagttcccgcagctgcgccttcagatccgagttcatctccagctcgagcagcgcctgagagatgccagactcgaactcgtccggcttctcgccattgggcttcac

SEQ ID NO: 30 - DNA, 435 bp, rat
  misc_feature (1)..(435): Clone #32 (MLSSH)
acctcatcaccactcacgccacctagtatttgggttttgatgagcagctgtgactccatgtgtctcacggcaaacactcttctcttcctcatttttttctcaaagacgaatctggagggagtcaggcatctaggatatgctctgacaacaaaatggatggtttttgggcagagacttccaggggtcttcctgacatttggatttttccatttgtgaaaaattaggtgttgcgtatgttaaaggtctggacggtgctcttccaggaaaggctcaaacttgcttagatcactagctgtgagcaacggagacagtgaagaacagtgcagctatgtcttacagagtgtcggccccagtcccttctcagaatttgagttccaagacttggatgaaaagcaaaattcgtctctctggttatagcccccaatcctcttgagt

SEQ ID NO: 31 - DNA, 468 bp, rat
  misc_feature (1)..(468): Clone #33 (MLSSH)
actcttagccctctccaggttcagctttatgaagattttgctaagtcgcgtgccaagtgtgatgttgatgagactgtgtctaactgctgcactctcagaagaaactgaaaaaccaaagctgaaagctacaggtcacgtgttccaggctttgcaatatttacgtaaactctgcaaccatccggcattggtcttaacacctcagcatccagaattcaagaatactactgaaaaagctggcagttcagaattcttcccttcatgatatccaacacgcccctaagctctcagccctgaaacaattgctgttggactgtggcttggggaaatggcacttcttcggagagcggcacagagtctgttgtggcacagcacagaatactgaatcttcctgtcagcttgaaaagcatgctcgatataggtagaacatgatctgctcaagcctcacctggccctcttgtcactttactt

SEQ ID NO: 32 - DNA, 196 bp, rat
  misc_feature (1)..(196): Clone #34 (MLSSH)
accaggacgccaccgcagaagaggaagaggatttcggagaggaggcagaagaagaggcctaaggcggaagctgtgcttcacctcaggctgcttagttcccttagcttttttccaactgccctttgttttctttctgctgcctctgtcctgtttttgttccgtttggtttctccattggggggtaaacggtgcctgg

SEQ ID NO: 33 - DNA, 435 bp, rat
  misc_feature (1)..(435): Clone #35 (MLSSH) n is c, g, a or t
acagacagtgtctgaaggaagcacttaaagtataacaataaataaaacctaccatacccctgccccatatagtatatgtccacccaagaaaggatacccaggtcctgggctgtaaggactatccacttcagtaagtttgagagaatcttggagagtgctcaagtcaccaagtgaaactgttaaaggccctgaacaatgacattcccacccaacacccccagcacagtcctcacttcctgcagcccatgggctgttctcctttccctgggaggaagatttgttgggcccacgctttgctttggggatgaagcaggtcttagacaccaagggatcaggccaatnntggtaacttcttccatgacctcaagctggccaggtaggctgggcccaacatatgcctgtcattcaggggcaaaatgaagaggacttatgagt

SEQ ID NO: 34 - DNA, 758 bp, rat
  misc_feature (1)..(758): Clone #36 (MLSSH)
gtaccacactgaggactcactcttcggagaacctactgagttcgaatatttgcgaaaaatgctgtttgaatatatgatgggtcgagagaccaagaccatggccaaagttataactactgtcctaagattccctgatgatcaagctcagaaaattttggaaagagaagacgctcggctgatgtcatggctccgaacctcatcttgaagaaatggggagactgaggctgggcatccgctgctgaggaagctgccctcccacatgtggtgtttgcggagaatggcgctgtcatcatgggccattcgtgtagcttgaagacacatgctaccctggccacagatacatagcttttgaagcagacattcttggtggggaaatggtgatagttttgttctttttcattcagtttttttctgggaagttctctgtggttcaaaggctgttctgaccggccacccgacttggctgttcagtgtctttcttgggttcttctccctctctttaaagaactatcttccttatttattttagggtggtttctttttcttcttaaagacttgtgcaatacattttgaggtgaaacttagtgaatttttttctgataaattagagaatttaattgaatatttgctaaggcttgcttcttcaagctatgataagtcccagatggcagcacctcatgtttacctagcttttgtacctcggcgcgaccacgctaagccgaattctgcagatatccatcacactggcggccgctcga

SEQ ID NO: 35 - DNA, 209 bp, rat
  misc_feature (1)..(209): Clone #37 (MLSSH)
acaggcacagagatttccacgccgttggccaccgactgcttcttaaactgacccttggccttcaccatgatctccactcggctgtgggagaatttctcaatgactgactcgatccagatcagaggtttgacctgggtgctgagacggtaggacatgagttcgaagtcccatccgggggtatgaaggagatggtgcggtcgttgtcgaac

SEQ ID NO: 36 - DNA, 268 bp, rat
  misc_feature (1)..(268): Clone #38 (MLSSH)
caagctttttttttttttttttttttttttttttttaacagtttaacaaaactctttaataaaattcataaaaatgcatcttagagaacagttccgattgcttgccttttccactccccacaaaatacaaaattatyagcacccacacacatatacactcaaactacatacaaagggcacgctatcaacattagacaattaaactacagagaaaccaacagttctcacagttgatgcagtchtcatttgcttccaaaattatgtgagt

SEQ ID NO: 37 - DNA, 590 bp, rat
  misc_feature (1)..(590): Clone #39 (MLSSH)
acaagctttttttttattttttttttttttttcttttgtttctttccagcattaacataaaactttattgaagatgcacaaacaaaatcaatgcaaaggacaaaagggagcctacttcgggtgaattggttcactgtcacttacggggagggaacagtaaaaatgtttcaatgtgggcagcaagataaaatgcaacacgtttcaggacaagtagtgaaatacattcattcaactcaaacctgaaaacaaacttggctaaatatcaatcaacattgatgatacagtatgatattcatcattcaaaattggcagctgaaagggttcctttgccatataaacaaagtggagataaagaatagtttacaaggtgtgttgctgctatgctacaaagccaccaagttattcctggctctttgggaacatccgctcagtcagtttggtcacaaactttccaaccttgactctcacctggaaatcatggtggctttcaattcccaggactttatcagcacacacttgaaattcttttaaaaagaaaaaaagggaacccccactaaagtgattctgagcaggctcgttttcttcccatt

SEQ ID NO: 38 - DNA, 295 bp, rat
  misc_feature (1)..(295): Clone #40 (MLSSH)
taaattcttgtcagaatgtcttaatcgatcatttgttttaatattcttttctaaagaacacttttttaaagagaaactagtatttgctaagcattagtaatctcgtcgtcttccttgatgactgtaatgcttccaaaacaaatgccttcagccgtaggaatggcacagcttgtgaagcagaggccaggacaggtattctgggttgtgttccactgatatacacataaatactttcattgtgagaatgtatggttgattaaacctgtatgtgggtgtgcaagccatttttatagtt

SEQ ID NO: 39 - DNA, 639 bp, rat
  misc_feature (1)..(639): Clone #41 (MLSSH)
accaagactgcagcaaacaaaacaagttaaacttttgaggtcctcattgcctcaaggccctgacctgtattttgcacaggaagcagagcagtggttacaagccaatgggggaagtgtcgaatttccaccagcccttgcttaaaactagtttaactccacagtccatttcctagcatctctgtgtttctactctctgcattatcgtaatctgagcagtccaccttttaccatatcagttggacctgatacagcaagtagaaatgattcttatttataaaaaaaattcttggtagtttgtgttgttttaaaaaccagaagcagattacaaaagttacacttattacatttttaactgtaacataggtgtgtgctcatttggacattttttggcttatttctacactttcaaaaattgctataattcactttgtccctctatgatagaaatactatattagtattttgtaaccaaacgcctcctttgttaaacctggccctgagtttgacctgagtataaactttagaacttacaatgccacagagaatgtggctgctctggtgacctgagtctggtttcctagagatacaaggctgcagcgccctttccagggcttggggatcaggaagccagaatttacc

SEQ ID NO: 40 - DNA, 214 bp, rat
  misc_feature (1)..(214): Clone #42 (MLSSH)
acaaatgctgtatttttttacactgtcaacttattcvrmttgagaaaatgacttttttatagtaaagtgtattttggtaaattctggcttcctgatccccaagccctggaaagggcgctgcagccttgtatctctaggaaaccagactcaggtcaccagagcagccacattctctgtggcattgtaagttctaaagtttatactcaggtcaaac

SEQ ID NO: 41 - DNA, 779 bp, rat
  misc_feature (1)..(799): Clone #43 (MLSSH)
acagccaatcttaacacagccaggcttctgcgttgctgcacaaaatctcttctgcaaagtccacggcatctcagatctcctaccctagccaaagctttcaaactgaggcacttaagaacacaaccttaactgtcatgctttccagccagccaacactgtcttcctcctaaaagcaccacagcatcacccaggagcactgatcagcccgagaggctttgctgagtgcagggagcatgctctgccctaactcagaggaatcgagtcttgggccagcacccacaccacccggaccatttgttttagaacaaacgaggagacagagagcctctctgagccagggatgtttctagaaagtcggccaaacttttcctttaaagtttaaagaatatcagaggcctgggagggatagaggtggagaatctgaaagccaccggaaagggacagccttctctacctactgcagatagaggttgtatctctgctgggaccggacatctttactgaccgtaaatatccaggacgtttctgcctagagcaagactaaacgtcaaagaggaaagaagaaataaaacaaaacaaccaccacaaaaataccaccccgaaacacaaaaaaatgggtagggaaagagaggcagatgtggtagagcaaagagctttcagccatgtatcccagagtcctacctggtcacagagctagtgaggaatgaggcaacagcccagggaaaagccttaccacaaagcacagcgggaccaaagggagctaaaacctaacctgctcc

SEQ ID NO: 42 - DNA, 679 bp, rat
  misc_feature (1)..(679): Clone #44 (MLSSH)
accttaggtgggagctcagaaaacctcaagatgtcaccatcaacaggttccagtgaatactagcctgtaattttaaacatctagcagtaaactattgcagttgtaatgtaaaggaccctgtttctgtagaaaagaaaacatttaacataatggttgtgaaatgtaacatgaagcaactaactagtgttttgttgtttttaaaatcttccaggtttgtctttttttccttttttttaaagatacaatgatagaatttttgccaatttaaaatcttggtttaatttaatatattaatttgttcatgcagaaataacaccaacattaggaatgctagggagaatgaattgcagtttttataacaatttttttaagtttggtattaaaacattcatgtgtctgtcccaaaaattgtaatcacgttcaagtgcagtcatttgtggttataatcttgttttgtgtgcttccattacttaattcctcttaaggacacagagattagatggaagctcaatttatatcaaatttacattgtattgaatagacccaaaaagaactaaaaggagactttcacttggtgttggtcagatcataaagtgcagtgttctgttgttggaactgctccattcacaaacagctttgcagaggaaaggagctggcttaatgcagcagtttatactgt

SEQ ID NO: 43 - DNA, 218 bp, rat
  misc_feature (1)..(218): Clone #45 (MLSSH)
accattcccccccctttcccttctgaattgagccaaaattggacatcagaaagccgcctgtgtgccccgggagctggggagggggcgggggctactggtttccatttcctgtgctttgatctgcttgacttgtattggccctgagaaggcacccagataccaacactgtcaaaggtggccaaacgtcttttcaagaataattgctttcagcatgcaga

SEQ ID NO: 44 - DNA, 589 bp, rat
  misc_feature (1)..(589): Clone #46 (MLSSH)
ctcagaattctcagaaagccaaagatttctttttttaactgccttcagcagtgagcacaaagcctcatttcaaaggagactgacaaattgtccccagctgccagaaggagaatcccttcctggacgctttcctgtctccagtgagttgtcacccaaccctgctgcaagaatgaaactcatcaagttcctaggaggtgtggtcttcttcaccctcatgttctctggctactctgagcaaaaccaagtgaatgtgctgtgctcaacagattggttcatggtcaccgtccatcccttcttgctgaataatgatgtgttcgttcacttttatgaggtgcacttgggccttggctgtcctcccaatcatattcacccacatttctaccagttcaactaccgagttactgaatgtggcatccgcatcaaggccgtctctccagatgtcgttatctacagctctgagattcactatgcttccaagggctcatctgcgagatatgtgattcctgtgtcctgtgctactcctcgccggtccccttggctcactaagccctactccgcgaaagcactagcagtaacatgggcgcaaccc

SEQ ID NO: 45 - DNA, 223 bp, rat
  misc_feature (1)..(223): Clone #47 (MLSSH)
acaagcbtttttttttttttttttttttttttttttttaatttaaaaggcccactttattaaacacattctttctttcagaatcctgatttttaaaaaacctttatctactaaaaaacagacatataaatcaagcaaacagttaagactacaggaatggggctcctagtgacgctgaaggaggcgatargtggggaagggcagarggctgtggggagggaggt

SEQ ID NO: 46 - DNA, 271 bp, rat
  misc_feature (1)..(271): Clone #48 (MLSSH)
acaagcttttttttttttttttttttttttttttgtttataaaatagaattttattttgggtgtttccaaagactcattctaggttgttcacatgatagctattcagaggtctagaaaggtcatcatgtccagtaagaaactccttaaagggtaagattctgavtcctttggatctgtcatgtcaatcagtagctgtgattttkkgtccagacatcatccaaaaaagaacaagtbctgagccatctttaactcaggcccagatgaaacagc

SEQ ID NO: 47 - DNA, 201 bp, rat
  misc_feature (1)..(201): Clone #49 (MLSSH)
acaagcttttttttttttttttttttttttttagcactgcaacactttatttagcgtattggcgtggattagctgaagttaacaatcgccaggaagccaggtcagctggactgcataatacycgtcttcatttattgacattacttagcaatttattggacaaaggtcaaacttttgtttttattaagcacattccacagt

SEQ ID NO: 48 - DNA, 295 bp, rat
  misc_feature (1)..(295): Clone #50 (MLSSH) n is c, g, a or t
accaagactgcagcaaacaaaacaagttaaacttttttaggkcctcattgcttcaaggccctgacctgttattttttacaggaagcakagcagtgktttacaagccaatgggggaagtgtcgatattttcaccagcccttgtcttaaaactagdtttatactccacagttccamtttcctagtcagtctctgttgtttttctatctccccgtcaattamtcgttaaatcagtagtcagttccaccctttttaccaaantcagtttggtatcctgtatatcagtcaagttagcaaa

SEQ ID NO: 49 - DNA, 404 bp, rat
  misc_feature (1)..(404): Clone #51 (MLSSH)
acctgctgctgccacatcctcttgcctactacccagggccagccactcaactctacatgaactacactgcctactatcccagccccccagtttctcccactactgtgggctacctcaccacacctcctattgccttggcctccacacccacttcaatgttgtcacagccaggagccctggtccgaatgcagggggtcccctacacagctggtatgaaggatctgctcagtgtctttcaagcctaccagctggctcctgatgactacgccactctcgtgcctgttggtgacccacctcgcactgtgttacaagcccctaaggagtgggtgtgtttgtagaagaggaaaccaggaagcaggatgagctgacgttcccagctaacaagcctttcctgagtccagagt

SEQ ID NO: 50 - DNA, 440 bp, rat
  misc_feature (1)..(440): Clone #52 (MLSSH)
acttcagtagtcaaggaaagaagagagactgcagccctgtcaattattttggtttatattagtttaagagattatttttcttggaaatagtataagcaagccatgcataaaaggaacagtgtattccatgaatatctggaaatttctcattactataggtaggggtgtagtctgggtaaaatgaatatttggagctaacaggtaatttatccagtgattatatttccttaactaagtcatagcaagacttttggagtatgtgcatggtattggccactgaggagcaggacccatctggcatacaatgaggagcctgcctcagcattccacatctcagtctcttcactgctctcacaaagaacttgagtgccagtcagagctgagtcaggttccacagtgggctaatcatctttcaggctgtcgaaggaagcatcagcatt

SEQ ID NO: 51 - DNA, 224 bp, rat
  misc_feature (1)..(224): Clone #53 (MLSSH)
acaaaggaagaaataccaacggacagctgacgagaaatgatggtccgagctcccgtgctccggcggctgtcaacacaggagcatgggtgcacaggagcacgagtggtggtcgtcggagctgaatttggagtgcagttctacttctacatgaactgtaatctaamcctaaaatgctgatgcttccttcgcagctgaagatgattagccactgtggaactgatcag

SEQ ID NO: 52 - DNA, 359 bp, rat
  misc_feature (1)..(359): Clone #54 (MLSSH)
acaagctttttttttttttttttttttttttttaaacattggtgataattcaggtccactgccttttacccagctcaaaaataattttttagccaataagaaacaatgtagatacagcaacttgcctcatttctgatatgttaaaaggatttggtatctattaaagagaacaaataagtatttcactgtaacaaattatgaagcaagtaatttttatgggttgtcttataataaaactattttggtattagactattttggtattagatccaaattatagaaagtaaagatcaaacctctctcgaaaataatacagcattcatactgacatgacagcttatgagagctagatattacaa

SEQ ID NO: 53 - DNA, 283 bp, rat
  misc_feature (1)..(283): Clone #55 (MLSSH)
ataagaaacaatgtagatacagcaacttgcctcatttctgatatgttaaaaggatttggtatctattaaagagaacaaataagtatttcactgtaacaaattatgagcaagtaatttttatggttgtcttataataaaactattttgggtattagactattttggtattagatccaaattatagaaagtaaagatcaaacctctctcgaaaataatacagcattcatactgacatgacagcttatgagagctagatattacaaataattaaactgcattaaaa

SEQ ID NO: 54 - DNA, 628 bp, rat
  misc_feature (1)..(628): Clone #56 (MLSSH)
actgacgggcagcttcacgtcaggcagatcttgcatcccgaggcttccaagaagaatgtattactacctgaatgcttctattcctttttcgatcttcgaaaagaattcaagaagtgttgccccggctctcctgacctcgacaagctggatgttgcagccatggcagagagcctaaactttgagaagaatgactccatgtccagatacggagcctcccaagttgaagacatggggaacataattttagcgatgatctcagagccctacaatcacagattttcagatccagagagagtaaactacaaatttgaaagtggcacttgcagcaagacggagcttatcgatggcaacacggtggtgagggctcgaggtttgccctggcagtcttcagatcaagatattgcaaggttcttcaaaggactcaatattgccaagggtggtgcagctctttgtctgaatgctcaggggcggaggaacggggaagctctggttagatttgtaagtgaggagcacagagatctggccctgcagaggcacaaacatcatatggggaccgttacatcgaggtttacaaagcagcaaggcgaagactttcttaaaattgccggagggacatccaatgaggtgg

SEQ ID NO: 55 - DNA, 368 bp, rat
  misc_feature (1)..(368): Clone #57 (MLSSH)
acaaaaatgccaggaactgggcctcttctgtctttggtgtttgttaacagggagaagattaacccccagtcctctgtaaagccaagtgccaagtgaaaagaaccagacactttgcagactctgggcctagcagctaggaggcctagaccctggacagattgctccaccctcaggccactgtcagcagtaacgaaccgtcatgtataacaatggagatgcagactgaccgtctctgtccctgcaaggacctccagtgtagtctgcttcccttcacctccgagaatcacaagggaggtgatggtagtgaacctgacttttcacattgaaagcacataaaactattagatttgtaaaaaaaaaaaaaaaaa

SEQ ID NO: 56 - DNA, 371 bp, rat
  misc_feature (1)..(371): Clone #58 (MLSSH)
accgagcaccttagattacacattgttgaattaaactattggaattaatatatgtatcatgttatttacaatacataagtataattctcttgtataacagtgatttctgaattccagtgatttctgaaaatatataatgtgaaagaatacaatacaatttacaaatgcatcagcactttcctacaggtttgaagctggcctccactcttcaacattttaaaataggaaaccaaccaaaagtcaatgtcctatggtggtatttcctcaagcatcacctataaaatccactagtttcacagatgccccattatgaagaagtgcaaggcatagagccctcaccccccaccttcaagattatgatccatgagacc

SEQ ID NO: 57 - DNA, 207 bp, rat
  misc_feature (1)..(207): Clone #59 (MLSSH)
ttttttttttttttttttttttaacattacagtaaacatttattaagctgcaatgttcacaatattttgatccaattcttaaagtcgatctacaaagacctctaacatcaagccagtgattaataaataagaggaatgtatccatcactttgcgaaaaaatactaagcccgatgctttttttccctatatattcgtagaatcagctt

SEQ ID NO: 58 - DNA, 339 bp, rat
  misc_feature (1)..(339): Clone #60 (MLSSH)
accatcattcacaggaatgaacaaacccaaacacgataaaatttaaattctcatgtaactgctacacgtgaatgtaaatgaactaattatattatcatacccttcctttaatcataagaaaaagggaatttacatggcataacaaaaaatacacaaaatttaatgaaaacacaattctcttaaaatctcacaaacttactttaaaggatgcagaacgcacttgaaatgattgacttaagctgattaagtttttttttttggtttttttttttgtttttttgtttttttttttttttactgcaaactgtttaaacatcagcttaaccccacatgcatggt

SEQ ID NO: 59 - DNA, 363 bp, rat
  misc_feature (1)..(363): Clone #61 (MLSSH)
ggtacaagcttttttttttttttttttttttttttgcaaagaataatcactttattaattgagcacgattcagactggtctaaggacacaaagcatccggccaactcataagagtagctgaagtgatgagagtttttgtggccataggtccgggcagccatcacctgcaacatccactcccagaagcactggaggccgggcgaggacacctcagactcaggaggccgggaactctgtaagaacaggtgttccaggcctgagtgccgccaaccagcttggctggaacgaatgctgtgagaacaggctggcatcacaaaggtaccacctgaagggtgggcagagggcctcggggaggagctacgt

SEQ ID NO: 60 - DNA, 538 bp, rat
  misc_feature (1)..(538): Clone #62 (MLSSH)
acttttcagctgagggaggggatcacagtctacagactcatttacttatattagacaagattaacctcattcaaaacttactgcatgtttataaattcacacaaatacagaaattgatgcaattaaacaaaatcttgaggatctttttaattcttccattataactttttctgcaggctcaaattttctgcttcataatcaccaatggttatggttcaacttaactacatcaattataaacctcttatagtcttaaagaaatgtaagtgaaaattacagtttctttccaaaaggtttagggttcttcaaatttcaaatgtgtctccccctcccacctcttcagacatctcaaataagctaaaaataacccatctcacctgcaacgttcagtaacagcaatcacttgtataaaattttaactattattttaagacacaaaaaaatggctgtctagcaatctgcctgtcacaagttagaacactacacttaggaatcaacatgagggtttcaagtttcctccagtttaggcatttatacc

SEQ ID NO: 61 - DNA, 688 bp, rat
  misc_feature (1)..(688): Clone #63 (MLSSH)
acttgggctttggagctcctcacctgtgaaattttttttctttttctttctttctttttttctttttttttttttttgtgggttttaacctaagacccttgtcactggacaatttccgataggttctgcctggcatcagcaaatttcagccagttgcatcctcctgtgttttgctctttggtggtccctccgtagctaggacagacaaggagaacctgtctttctgtcctggtggcagaagaagcacattcttattgtcacgcggacagctatagcagccgatggcttgaccgactctccgcccaaggtctctgcacatttcaaacctttgctgttgtgtcagcttcttgtctgcatctgtgataaatctctgaatatcctgtaattaaattcatttcttgtgacagtgattagtgtgcagttaaatcccccatgaagtaattatttccctgtttttagctagggattttttttttttttaaaccaatgacttcttcacagtggacttcctgttttgtgtctcggcttagacgttttatatagtgtttagtttgacacagtagaaagccattccgagtgaacatggcggggcgcgtgcgcacacacgcacatgtgcacacgttagttgggaagctgattctacgaatatatagggaaaaaagcatcaggcttagtattttttcgcaaa

SEQ ID NO: 62 - DNA, 200 bp, rat
  misc_feature (1)..(200): Clone #65 (MLSSH)
attccgtcctgcgtgtctgttctgcggaacagtaggcagttgttttccgtccggcttctctcacactcaagtgcgcgcctccacctcatggaagactcgatggacatggacatgagcctcttaggctcagaactaccttttcggttgtgaactaaaggctgacaaagattatcactttaaagtggataatgatgaaatga

SEQ ID NO: 63 - DNA, 215 bp, rat
  misc_feature (1)..(215): Clone #66 (MLSSH)
acccacacagcactgctctgtctggcwwwrgcctaagtggctgaagctgtagccatgtagctgaaaggcctcccgcttaggcggggtctgagctattgcactatcacactatatttctgcttccaatcatggttcagagtcctagtgagagtgtaaacaaagatgaaagrvvtgttactccactttaatatagtctgtagtatcatacacatcgg

SEQ ID NO: 64 - DNA, 403 bp, rat
  misc_feature (1)..(403): Clone #67 (MLSSH)
acaagacctagaagccacatttagactccttgtggctcttgggacacttatcagtgatgattcaaatgctatacaattagccaaatctttaggtgttgattctcaaataaaaaagtatgcctctgtatcagagccagctaaagtaagtgaatgctgtagacttatcttaaatttgctgtagcagtggggaaaggaagctgaggggtttggttttttttgttttgttttgttttgtttttttccccacactttacatgactgattgtagatatttaaaaagaaagaaagaaagaaaagaaaactggattaagttcagatattataaagttgtgttgaggagatacaaattataaataaagtttttgcactgaaaaaaaaaaaaaaaaaaaaaaaaaaagcttgt

SEQ ID NO: 65 - DNA, 292 bp, rat
  misc_feature (1)..(292): Clone #68 (MLSSH)
acaaataataagcacattttaaatgctgaccagcaaaaatcttttgctaagtgtccaaacgtgttgatcatacctatctgaggttttccaacttgtttgaattatggttttccccttctcttcccaatctcttgcaaaaaagtaaaagtgggatctgctagtgaactgagcagaaatattttatacgccttttgagctatgtaacttaataattggatacttgatcatttgttttattatgtaatcgataaaatggtgatgtgtattaatgttagttcaaccataaaaaaam

SEQ ID NO: 66 - DNA, 380 bp, rat
  misc_feature (1)..(380): Clone #69 (MLSSH)
tctagatgcatgctcgagcggccgccagtgtgatggatatctgcagaattcggcttagcttttttcgcggccgaggtacatgtatatgtatttaaagactgtttactgcagttactcaggaactgcttttcattcacgttaagctgctttcaaaatttaaaaaaaaaaattaaaagaaaaaatacttaaaacggtgcattgataaaatctaagagttttttcagttttgttttctaggtaaatttttgcgtaagttttgtggcacagggtagccctaagtatccctcctcggtcccgcactgggatcctcaaatgctcctgaattctagttgtttctttcattggcacgggggagagtgctttaaggcagatgaagcgct

SEQ ID NO: 67 - DNA, 714 bp, rat
  misc_feature (1)..(714): Clone #70 (MLSSH)
gcatgagactgagacagctcaagaggttcagaagagctgacgaagttaacagactatgtttagttccaatcgtcagaaaattttggaaagaactgaaaccttaaatcaagaatggaagcagcgaaggatacagcctgtgcacatcatgacttctgtgagctcattacgcgggactagggagtgttcagtgaccagtgacttggattttccagcacaagtcatcccgttaaagaccctaaatgctgtcgcatcggtgcccataatgtattcttggtctcccttacaacagaattttatggtggaagatgaaactgttttacataacattccttatatgggggatgaagttctggatcaggatggcactttcattgaagaattaataaaaaattatgatggaaaagtgcatggagacagagaatgtggatttataaatgatgaaatttttgtggagttggtaaatgctcttggtcaatataatgatgatgacgatgatgatgatggagacgatcctgatgaaagagaagaaaaacagaaagatctagacgatagtcgagatgataaagaaacttgcccacctcggaaaatttcctgctgataaaatatttgaagccatttcctcaatgtttccagataagggcacagcagaagaactgaaagaaaagtataaagaactcacggagcaagcagttccccggtgctctacctcctgaa

SEQ ID NO: 68 - DNA, 389 bp, rat
  misc_feature (1)..(389): Clone #71 (MLSSH)
actgggtataaccgagagctttcaagtgaagcggcaggttcttttgagtgcggcggaagcagcagaggtgattctacgagtggacaacatcatcaaagcggcaccaaggaaacgtgtccctgatcaccacccctgttaagcattcccatttgctgttgaactctggaccagttcatagcaaagctgtatttgaaagacttcaccctttaaagaagactatggaattgctgtgtccatgacagccttaagtttgaacatttagctgaccttctgtgttaaacatgggtctaatttatttgctatttcattttccatacagttcagttgatttacaggttcatttctcatactgtgtattaaaataaaaaatcagttacttagtccttaat

SEQ ID NO: 69 - DNA, 527 bp, rat
  misc_feature (1)..(527): Clone # 72 (MLSSH)
acaaggaaaagttcgggtttgatccattcattagcagtaacattcaatatttcctggatcggtttttataccaaccgcatttctttccgcatgcttattaaccagcacacccttctgtttggtggtgacactaaccccgctcatccgaaacatatagggagtatcgatcccacctgtaatgtagctgatgtggtgaaagatgcatatgaaacagccaagatgctttgtgaacagtattacctggtagctccagaactggaagttgaagaattcaatgccaaagcaccaaacaaacctattcaggtagtttatgtgccgtcacatctgtttcacatgctatttgagttgttcaagaactcgatgagagcaacagttgaactatatgaagataaaaaagaaggctacccacctgttaaaactcttgttactctgggtaaagaagacttgtccattaagataagtgacctaggtggtggagtcccacttcgaaaaatagaccgtctttttaactacatgtacctcggccg

SEQ ID NO: 70 - DNA, 201 bp, rat
  misc_feature (1)..(201): Clone #73 (MLSSH)
acccatgccagtccatagcaattctactgttttaccaacctttttctctacctcctgtttgcgtgatgttccttgtatagtgccactggagahatgccccaacggtatgccgccactaacgtttctctgttgcataccttatgttttaccattaaagactcttccctgccctgtatttggttacgtagagccacattagct

SEQ ID NO: 71 - DNA, 227 bp, rat
  misc_feature (1)..(227): Clone #74 (MLSSH)
cttacctcactgcttttcctagvdddgcacctcctccgccagcttcctcctcagaccgcttttcccgcgagccgaccggttctgtcatgtcgaccctcagtcccagcgtcgtgattagtgatgatgaaccaggttatgacctagatttattttgcatacctaatcattatgctgaagatttggaaaaggtgtttattcctcatggactgattatgggcaggactgaa

SEQ ID NO: 72 - DNA, 189 bp, rat
  misc_feature (1)..(189): Clone #75 (MLSSH)
accagctgagacgctctacgatgtctgcacgacatagaataccgaaagawgtgggacagtatgtcatcgagacattcgacatcgctcgactgactgtcaacgctgacgtaggatattactcctggaggtgtcccaagcccctgaagaaccgkgatgtcatcaccctccggtcctggtccccatgggcgc

SEQ ID NO: 73 - DNA, 616 bp, rat
  misc_feature (1)..(616): Clone #76 (MLSSH)
acaaaatggacaaggggaaacatctattgtcctttgaagacaccatggccccggcatgagccctgacatctcctcaaacggttgccaggatgatgtgtcttatgtattaggtggatgacgcggttttgtttgtattctctttatgtcagtatcgggcatccatgttaatgatccacgagcggctggctgtgaagttacaccaaatagaaatgttcagccactttgataaagtaaggctgtggggatgggagcctcccaggggctgtagagacagaactcaaaagtgcattctgctgggaaccaaactgctaccaggaggttgtgcccactaagacaggaaagtccctggatatatgctgtggcaaagttagcaagggcaagaggacaagagacccttttttgggcactgagggaggacgcacattccatcactcgattaagttggattagcctcctcgtgagccagctccagcatcagccatgagccgtgggccaaatccgtgaaggtgaccccctccttattggtttctgctcatttttagaaggagaaatttacctttcctatttattttcaagcattaagagaaatagcccccctgtctggtttgttttgttt

SEQ ID NO: 74 - DNA, 555 bp, rat
  misc_feature (1)..(555): Clone #77 (MLSSH)
actctgttagggggaaccacagctgatgccatgtgtcctatcctagagtttgaagcagatcgccgagctttcatcatcaccatcaactctttcggcacagagctttccaaggaggaccgtgccaagctctttccacactgtggccgactctaccctgagggcttggctcagctggctcgggctgatgactatgaacaggtcaagaatgtagctgattactacccggaatacaaactgctttttgagggtgcaggtagcaatcctggagacaagaccctggaggaccgattctttgaacatgaggtgaagctgaacaagctggccttcctgaaccagttccactttggtgtcttttatgcctttgtgaagcttaaggaacaggaatgtcgcaacattgtgtggattgctgagtgcattgcccagcgccatcgcgccaagatcgacaactatatccccatcttctagagttccgacttaaggttcccaagtgcactgtgtgtgtctgtgtgctgtggtgaagcctctggttcacttgtctgacttggtgaggtgtag

SEQ ID NO: 75 - DNA, 523 bp, rat
  misc_feature (1)..(523): Clone #78 (MLSSH)
tagcatttaaagtgaaagctttttcttcctggtttgctagtgttctgcgtgtcttctttagttgaaataggttagtggagaagttaagaaagcggtggccgtgttacgctggtggtgaaggccagggccgcttacaaactctcagttttgttttgttttgtttttttataacagtcccactctgacaataatgagttcactccttggataaggttgttaatgagggaagagcccttcagacctaatcagctcttactggacccagctgtcaacactgttgcattggggattaagtttccaacccgtgaatcatgggacagactcaaaccgtagcactgggcatgtcgcctaacaatataatgtgactcagtattctgtgagacatgggataatattcctgacctacctccacgggtaggtgcgagcatgagcaaatgctgctttataaaacatttggggatcctcagcagaggagtatttaagtgctgcacatttttatagtaaccgtccaccatggacttga

SEQ ID NO: 76 - DNA, 305 bp, rat
  misc_feature (1)..(305): Clone #79 (MLSSH) n is c, g, a or t
aaagcagcaaaggtccaaaaactgtctaanaatgaagtgctcatggtcaacataggatccttgtcaacaggaggaanagttagtgctgtcaaggctgatttgggcaagattgttttgaccaatcctgtgtgcacagaagtaggagaaaaaattgcccttagcagaagagttgaaaaacactggcgtttaattggttggggtcagataagaagaggagtgaccattagagccgactgtagatgatgactgaagattccacgttaaacaatgccttggatggagttggaatttcttttaatagccta

SEQ ID NO: 77 - DNA, 298 bp, rat
  misc_feature (1)..(298): Clone #80 (MLSSH)
gcagtgaccggggacggcaagtggatggtgatcatctgtagcagggcgtcaccagcaggcagccagcgacgcatcacagccttcagcagtggtttgccctccttgtccttgtcctcactgtccagcttgatgtccagcttctcgatcagcttggctgtctcctccttcctgaagttcatggtggcatcaaacaccttgaagatggggtccaagatgagctggcagaaggtgcggggaagtttcttcccgtctgggctattggctgacttgctgaacttgccgttggccgggtcaaagt

SEQ ID NO: 78 - DNA, 534 bp, rat
  misc_feature (1)..(534): Clone #81 (MLSSH)
actagtcttagatatggatatatacaatatacacacatgcacacaatcccgcttctctaaagctacctakbaaattcgaatccacgggagtatatcccacagtccactccctgcctctccccctcctcggctacttagaaaagactcaaaagcttggaggggttttaagtggcagctggtagaactgaagtggcaccaagcaatgccacatcttacacaggaagaggggaaaggaaacgaacaacgtcctcctaggacacaatccatcaaccgtgtggaaaaacccaagtttccataggcacactgaggcacttggctgactctgcacccctccccctaagtccactcccatttaaccagagactgctgagaagacccttccttcccaatggctaaatcccggttgtgagaagctatggcttctctaaccagctgtcacttggagaggcactagggcagaagagaaaaagaaatcaggtccccaacttaccaccattggtaagggcagcccagggagtttatcctcttctcccc

SEQ ID NO: 79 - DNA, 508 bp, rat
  misc_feature (1)..(508): Clone #82 (MLSSH)
actggtagagctgtggctcgaattccaagagttcgaggtggtggcacccaccgttctggccagggtgcctttggaaacatgtgtcgtggaggccgcatgtttgcaccaaccaaaacctggcgtcgttggcatcgcagtagtgaacacaactcagaaacgatatgccatctgttctgccctggctagcctcggccttaccagctttggtgatgtctaaaggtcatcgtgttgaggaagttcctgaactgcctttggtggtttaagataaagtttaaagttataagaataccaaggaggctgttcagctgcttaagaaacttaaagcttggaatgacatcaaaaaggtctatgcctcccagagaatgatagctggcaagggcaaaatgagaaaccgacggcggatccagcgcagggtaccctgcattatctattatgtaatataatggaatcatcaaggccttcagaaacatccctggtattactctgcttaatgtaagcaagctgaaca

SEQ ID NO: 80 - DNA, 369 bp, rat
  misc_feature (1)..(369): Clone #83 (MLSSH) n is c, g, a or t
taggtctccgccatcatgggtcgcatgcacgctcccgggaagggcctgtcccattcgtgtctctgccctaccgccgtatcttgcccacatggcgtaagtgnacgtctgacgacgtgaaggaacaaatttacaagttggccaagaaaggcctgactccttcccagataggtgtgattcgtagggactcacatggtgtggcacaggtccgttttgtgaccggaaataaaatcttgagaatccttaagtccaaaggccttgcccctgatcttcctgaggatctctaccatttgatcaagaaagcagttgctgtcagaaagcacctgakaggaacagaaaagataaggatgctaaattccgctgattctaatt

SEQ ID NO: 81 - DNA, 196 bp, rat
  misc_feature (1)..(196): Clone #84 (MLSSH)
actgagcaggattaccatggcaacaacacatcatcagtagggtaaaactaacctgtctcacgacggtctaaacccagctcacgttccctattagtgggtgaacatccaacgcttggttgaattctgcttcacaatgataggaagagccgacatcgaagggatcaaaaagcgacgttccttttgaaacgctttggcc

SEQ ID NO: 82 - DNA, 182 bp, rat
  misc_feature (1)..(182): Clone #85 (MLSSH)
actttggatctggatttgctgctcctttctttatagtaaggcaccagctacttaaaaaataaggatatttaattcattbttttaacagaatgaagaaagtttaagagatatgatctggaaattggattaaactcttgaactcttatactagaaaaaaatgtaaataaactaatgatataaat

SEQ ID NO: 83 - DNA, 20 bp, rat
  misc_feature (1)..(20): Clone #86 (MLSSH)
acttgtatttgggtggggag

SEQ ID NO: 84 - DNA, 195 bp, rat
  misc_feature (1)..(195): Clone #87 (MLSSH)
accgtcctcccattttgtttttggaagattaaatgctacatgtgtaagtctgcctaaataggtagcttaacctatgtcaaaatgtctgcagcaagtttgtcaataaagtttagtcctttcttaatcaaaaaaaaaaaaaaaaaaaaaaaaaaaaaaaaaaaaaaaaaaaaaaaaaaaaaaaaaaaaaaaaaaaaa

SEQ ID NO: 85 - DNA, 238 bp, rat
  misc_feature (1)..(238): Clone #88 (MLSSH)
acaagcttttttttttttttttttttttttttttttttttttttttttttttttttttccattcataaaactttttttccacttacatgaactttaatacatgtgttcttaacaattatgcttggattgttcatgaaaatttcataagacattaaaacaaagctagccgtcatctcaagttatttccctgttaactatttttacagcacatgcatgttaggcaagtatcaaaaagaga

SEQ ID NO: 86 - DNA, 163 bp, rat
  misc_feature (1)..(163): Clone #89 (MLSSH)
caggtactcaattctttaagaaatgacaaattttggttttctactgttcagtgagaacattaggccccagcaacacgtcattgtgtaaagagaaataaaagtgctgcagtaactgaaaaaaaaaaaaaaaaacaaaaaaaaaaaaaaaaaaaaaaaaaaaaaa

SEQ ID NO: 87 - DNA, 220 bp, rat
  misc_feature (1)..(220): Clone #91 (MLSSH)
acaagctttttttttttttttttttttttttgcagtagacagtattaatgttttattaatggccagctacattcgctcacacatgcgcttctaagatttatatccaaacagcatatggtatttacatgcaatggacagattagaacaccaaattatgaaccttggctgcggtaagactatatttagttccacattaaattaaatacacaatagtctttac

SEQ ID NO: 88 - DNA, 199 bp, rat
  misc_feature (1)..(199): Clone #92 (MLSSH)
acctcagctccgagagacttatccctcagagagtcatggaccagcacaagctcagcagggaccagtgggaggaccggatccaggtgtggcacgcagaacaccgagggatgctcaaggacagtgctatgctcgatacctgagattgccaggacctggaaatgtakgggatcactatttcgggtcaaaaccaggarggaca

SEQ ID NO: 89 - DNA, 218 bp, rat
  misc_feature (1)..(218): Clone #93 (MLSSH)
actatgacgagatcatcaatgctttggaggaagaccctgcagcccaaaagatgcagttggccttccgcctgcagcagattgccgctgcgctcgagaataaggttacagacctctgaccatcagtgctgcctcaggattcagaggatgcacccaaggcttctggagagcgtgtggtgaacccacctcttgtagactatagcgtctttctcctgagcaat

SEQ ID NO: 90 - DNA, 214 bp, rat
  misc_feature (1)..(214): Clone #94 (MLSSH)
acttcctatgggacacaaaatactgtcatttgctccaaactggcatccaagtgcttggtgatgaaggcggagatgactcacagcaagtctgggaggaggatgaaacccgagggggcgatccagaacaacgacggtctgtatgatcccgagtgtgacgaacaagggctcttcaaagccaagcagtgcaacgdcaccgccacgtgctggtgcgtga

SEQ ID NO: 91 - DNA, 218 bp, rat
  misc_feature (1)..(218): Clone #95 (MLSSH)
gtgaaagtcgtgtatgctttcaagagattgaagggtttgggggttttttttgtttgttggtyggttggttggttggttggttttggagttaactttttatacccacttcagatatttcaggcaaccacattgtagcaatcctaaggtggtattgaggcagggaggtggcattagtttcagggctggggagccaatttaaaatacctcaggctatttac

SEQ ID NO: 92 - DNA, 250 bp, rat
  misc_feature (1)..(250): Clone #96 (MLSSH)
acagagtctgaatggaccgatgtagaaacaaggtgttctgttgctgttgaaatgagagcaggatctcagctgggacccggatatcagcaccacgcacaacccaagcgcaagahaccttgaactgacaaccctggtgacagaaggaggttttttgctcgtgttggtgatttacccaaagccgcaccagatgcagtgtcttgagtcgtcttgcagaagaatgtgtctagatcacattgccttttttattgtg

SEQ ID NO: 93 - DNA, 254 bp, rat
  misc_feature (1)..(254): Clone #97 (MLSSH)
cgcaggtgatggataytgcaawtcgcttcgagcgcgccggcagtacagcgtctgactactgatacattgagttatggacaacttggagaaacttgaaggccaactagctcaggccaactttaaggtcaactgggctagggcaatcttgtggtactaagtcmcagttwcwtattccttttgtttaataacatcaaggttaaaacagatcatttgtaattgttagagacgagttgactttccaagttmgccaattc

SEQ ID NO: 94 - DNA, 563 bp, rat
  misc_feature (1)..(563): Clone #98 (MLSSH) n is c, g, a or t
cgggcaggtacttcttgtttgtgacctttacgtgttcttgttatgatgtagaaagaattcagctaccacccactcctgcaaccgagcactgagaaatgtggtaaggcagaagctaaactctgcgtcctctagatgtgacacacaggcttggtgcctgvggtctgctcagttggctgacactcctttgaggtgtgtgtgctgtggtgctaactgggactgagaggagcagagtcagtggggaggcctgggacactgtctactatcttggctttattgatttagcacaccaaggtctcctcactgtggtccctcacagaacatgctagtgcccctctcaacctcatgggtcctgcttgtgtggatgagaaagctgcatgtgtggcagagtcaaagcatttacaattgagatacagttttaaatgcttacctcccagcttaaacctgactacccaaggcccgtgctttttagcataaatgactttaacatggggttggggatttagctcagtggtagagcgcttacgtaggaagtgcaaggccctgnggttcggtcccmagctccg

SEQ ID NO: 95 - DNA, 281 bp, rat
  misc_feature (1)..(281): Clone #99 (MLSSH)
cttttatctttagaaattttatttttttaaaatccgaggcaaatgcctcacactaacacccttcattttcttgggacctcttcagttgtcagcctccccttctaacttcaggcctagggaaagggctagggcttgttccctataacattcacctaagataggtgctccccaattcccaagaggcagcaagtggagccctggaactctgagtagtagaaaccaatcctttatatttggggattttttttatatcaataaaagtggacttcagggaaaaaaaa

SEQ ID NO: 96 - DNA, 272 bp, rat
  misc_feature (1)..(273): Clone #100 (MLSSH)
actcctttcagcaatcaaaaataactcactcttattgctgtcaagtagcaaaggaaaactttactctcaagaacttcagtccaacaaggaagaatcagcagtcataagactgagatctggagtagactaaaaccgaagtctccacagatctatcagtggagcaacgtgtccatgtgctttcaaagaatgaagtagtgaaacaaagacccagttcgccactgtgatgctgcggtgttgctgacgatccagtcactctgactaggcactgaagt

SEQ ID NO: 97 - DNA, 576 bp, rat
  misc_feature (1)..(576): Clone #102 (MLSSH) n is c, g, a or t
acctgagtcaktgcagctcaagtatggttggwtctcagagtggcttaaaacatactaagcttgggtccgtatcctgattctggtgtcanaactgggatatgctctgaccataaggatttaaaaatgccctggtttcagaatgctaccaaaggttgatagcagttgtgctcaatttcccttagaggtttcccatctacaaagtaggaaattcttgctcccctcactacctcacaaggatacagagggtgcacgagagcgcactgggagtgttggcgttttgggtagaggacgctgtagaagtgcaagcatctgtcacagttctcaatttcagctgcagccgcgatcgaaaacggataattgaattttttgttgttgtaggaagctcttaatgtgattccgtgaggaagcatgtaggggtggagcaaacgttgatgactactcttattcactatggtcttctagcctacttgaaactggcggttatattatctaatttttcataggaaagaagaaatattaaggcttaattctgtaatgttcattkdgctcataattcattaaaagttcatacaaggg

SEQ ID NO: 98 - DNA, 520 bp, rat
  misc_feature (1)..(520): Clone #103 (MLSSH)
ttttttttttttattagaaacttcctttattcttctaaacaggtgaaaataagcaattcttatatttctcacttgtaagatttttaaattcttaaaaatgcaattttcttttcaaagcacatgccatctttaaaaaattctcagcaatatacatttgcacccaagaaatacatgcagcatcactgccgtctgacaatgtcctgcactaacccaccgactcctgcacatgtccgttctactttgggactcagaacacaggcttcagtgcaacacttatttccgtaggaaacacaggcccagtggcgtcttctgacaactgtttccgaatggctgagcacagcctccgatctgccttaaagcactctcccccgtgccaatgaaagaaacaactagaattcaggagcatttgaggatccgagtgcgggaccgaggagggatacttagggctacctgtgccacaaaacttacgcaaaaatttgcctagaaaacaaaactgaaaaagcgcttagattttatcagt

SEQ ID NO: 99 - DNA, 256 bp, rat
  misc_feature (1)..(256): Clone # 104(MLSSH)
gaattgggccctctagatgcatgctcgagygkcgcagtgtgatggatatctgcmrdarttcggcttabrtcgtcgcgcgaagtgtacagcttttttttttttttttttttggcgtccmcagcattayybmhchvcacatagaaaggagcatccgagcttcctatatgagagagctgaaagagagatctaggtccacatggtttgggtcattarrrvsccatcaacatgcgtatgctggtgatgtcgtaaagggcgg

SEQ ID NO: 100 - DNA, 215 bp, rat
  misc_feature (1)..(215): Clone #105 (MLSSH)
acaagtccctctgccccgtctcatgggtctcagcctgggatgaccgcatagcagaaggcacatttcctgggaagatctgacctggctccgcccacctctcctctgctctttkaccttctccccgatagaaaagggggacctcagtatatgatggtccctaccctgggaccctgaatcatgatgcaactactaataaaaactcactggaaaggttt

SEQ ID NO: 101 - DNA, 579 bp, rat
  misc_feature (1)..(579): Clone #106 (MLSSH)
gtccccctcccgctcacgatcccggtctcggtcccgctcttcctcccgctccaggtccaggtaggcacatggggaacctcagaaaacccgggagcactattcctgcccatgccccattgtcctgttgcccccacagaagccgcagccggtcacgttcctccagaagccgctcacgctccaggtctaggtccaggtccaagtcatactcccctgggaggcggcgtcgctccaggtcccggagccccaccccaccgtaagccacaagtgtggaagttgtcaaatatacccctgttgctaaagagtggggtggcacccctcccaccactgggccaacccacagccttttccctcaatgcttaactgactgactgctctcagtgctcaggggcctgttgtagaggttaaagcttagagagtgtctcttcattccagctctgcagccggcctgggttctaactcagcacctcccataccagactcaaggcttggagaagagaataaaggccaccagatgctggtgaagatgggtgagtagacccgaccagtgccagctccctgctaaccatatgttgggatact

SEQ ID NO: 102 - DNA, 236 bp, rat
  misc_feature (1)..(236): Clone #107 (MLSSH)
tggcttacctgcctgattacaggatgcaagagtgggcccgccgggaagctgagagacttgtgaaataccgagaactcaacggcctccccatcatggaatccaactattttgatcccagcaagatccagttaccagaagatgactgacgaaatactgagcagggcttaagaagcattacttattcgcccctctctgttaatttcctggttcctcagaacaccttattaaagtaattg

SEQ ID NO: 103 - DNA, 255 bp, rat
  misc_feature (1)..(255): Clone #108 (MLSSH) n is c, g, a or t
accgcgtcttcatcaaaaatgagaaagggcagtatatttctccatttcatgatgttccaatttacgcagacaaggatgtgttccacatggtgnttgaggttccacgctggtcaaatgcaaaaatggagattgctacaaaggaccctttaaacccgattaagcaagacgtgaagaagggaaagctccgctatgtggccaatcttgttcccttacaaaggctacatctggaactatggcgccatcccccaaatttgg

SEQ ID NO: 104 - DNA, 447 bp, rat
  misc_feature (1)..(447): Clone #109 (MLSSH)
acatgatgacatgaggttccaggcttgcaatgaagttcatatggtcctgggtcaggacttccagtagtgaataataagactggctgagcttgggataatccaggaggtcactgtgaggaatagaggggagcagtttaatgaaggtctgtagggcattttccaaggcatcatctccatagagacggaagactccaaaattgacgtagctcccactgagggcagccttcagcatggagaagcagatggagatacccttgagcttcaaagcatagacctgatcctttggcacctctcccccgtgtcaagataagattagccatacattgttatcatcttgctggtttctcggaaaagtaagataccactgggagatgaaacatcaaattggagtcgctgagatctgttatgaactaattcagccatcagcttgagcacaggtgtagtacc

SEQ ID NO: 105 - DNA, 270 bp, rat
  misc_feature (1)..(270): Clone #110 (MLSSH)
aaatagaatcatggttggagctgatgttggcgatttaagacctgctgactaagtgagtgaacaaaatttgctaatatcaattagtgccctgcatgcctttcctttaccctgactcattccttacgggtaggatcaatcttttcagtatttgcattctggttcaattagccagaaaaggaactttgtagtgtcagaatgtcatccaactgtatatttactttattgtaaataatggtgaacagtggtcaataaatagttttatattccttt

SEQ ID NO: 106 - DNA, 199 bp, rat
  misc_feature (1)..(199): Clone #111 (MLSSH) n is c, g, a or t
gccgccagtgtgctggaattcggctttcagcggccgcccggcgcaggtacaagcttttaancttttttttttcatgaagggagaatcaagccaccagattttnattttcatgccagaaataaagttccctatcactcccattttcttacataatacatgaaatacagagaagcagaaattcatttggtgaactatcagc

SEQ ID NO: 107 - DNA, 321 bp, rat
  misc_feature (1)..(321): Clone #112 (MLSSH)
gtaaagcttttttttttttttttttttttttttttttcttttttttttttttttttttttttttttttttttttttttttttttttttttttttttttttttttttttttttttttttttggatcccttttttttttttttttctttbcttttttctttccaaaaacaaaaagggaagaaaacccctttccccaaacctaaagttttgaaaaaaaacccccctgggggggcttccattttaatcccccccaaaccctttttcaaaggktttttgggrdtcaaaaagggttgggraaaaaaacccccccccccccccccvgg

SEQ ID NO: 108 - DNA, 613 bp, rat
  misc_feature (1)..(613): Clone #113 (MLSSH)
acaagcttttttttttttttttttttttttttttttgtaattaagtaacatttattttacataaaacaattttcaaagaatacatttaaaaaccatctagatactgattttatagcattacaaaaaaattgagaggcacaattgaagtattcaaataaaaggtgaaggtgaaggtgggggtctctacttgctatccactgtgtttcctgggactcctcctacaggagccttcccatgaggcagtagtgcactttgaataaatcgaataaatcacataaaatatatacttttatacagaaagtgttgctgtgtcatcactttcaattgcttataaaaaacttcattttgaattgcacaaaagttctctttaaaaccggtaattcccagaaggcagggcttagaactatatgtctaaataattcattacaaattaattagatctctatttctccacagataaaaaaaaacaatctgtgaagaattacatcatttaagtgggacatttaaaaattttttttcataggaaaataggtttatacatttgcccctagtcagctcaactttctccccccactttaaaacaaatcaaagggcgttcagaacttttcagacg

SEQ ID NO: 109 - DNA, 287 bp, rat
  misc_feature (1)..(287): Clone #114 (MLSSH) n is c, g, a or t
actctacaacttaaaattgtgtctgatgctttgaaaagtgtgctcatgcctttaaaaatgtttaaatgngatctacacttagaacgttgcaattggttaaaaacaaaatatgaagatattataacctaaaagaattcatgtcacacatattttattgggggtgatgtgcctttgatttaattggggacacttttagaaggaaagtttgtgtttgtgattatctttgaagagcttggaaataaaatttttgcctaattcatcattttccatgacaaaaaaaaaaaaaa

SEQ ID NO: 110 - DNA, 182 bp, rat
  misc_feature (1)..(182): Clone #115 (MLSSH)
acaagctttttttttttttttttttttttttttttaacaatacttgggtttatttatcgaaagaaaataaatattttaaagagtaaaaagmvtgttgaggatggaaaggtccaccaaacggaggccaggggatgggagaattccccaggggaatgaaaaataaaatkaggttgggtagagat

SEQ ID NO: 111 - DNA, 506 bp, rat
  misc_feature (1)..(506): Clone #116 (MLSSH)
acaagacctatcttgcaggcccttccatatttttgaccggtttggacttattgttgttccattgattgttgttaattgaacaggcgtatttcacttagcttgttggagaatgtttgcttaaacattccagccttccttccccgggcgcagttggattccgagttgctcttttggagagatcacacggattttaaaccatatcatggctgtcaccacacatgccctggacattggtgccatgacgcctttcttctggatgtttgaagaaagggagaagatgttcgagttctatgagcgggtgtctggagcccgtatgcatgctgcttatatccgaccaggaggagtgcaccaggacctacctcttgggcttatggatgacattttatgaattttccaagaacttctctcttcggattgatgaggtgtaggagatgctgaccaacaatagaatctggcgaaataggacagtcgacattgtgggtgtatctgcagaagatgcactgaac

SEQ ID NO: 112 - DNA, 274 bp, rat
  misc_feature (1)..(274): Clone #117 (MLSSH)
acttgggggttttggagggatgaggggaggtgaataattgtgtgccacagtaggaagtcagaggccaacctgtgggagccggttctccatgggagtccagagattgaactcaggttatgagctttggtggcaagtgccttcacctgataagccatctcaccagcgctgactcggtaatgtcttggtgaattttcattgacaaagtattcttctatgaaaacatgcactgctctgttaaaggcccgaatgcaaagggatgctttaggaagccgaa

SEQ ID NO: 113 - DNA, 488 bp, rat
  misc_feature (1)..(488): Clone #118 (MLSSH) n is c, g, a or t
acttcatccagccatgtatttgctctcttcctgacttaaaagaaggtcagctttctgttgctaatagggcgtgtactggagacatgatttaaacatcagaacacagtggagggacttgacttgtaggaaacaccagcttaagagcacaccgtggggcaggcatttctacagctgtgcttgaatttnatggggcttctcgcagcgaataaaaggctacagccaaagctgttattttagaaaactaaaaactaaaaattaaccttactatgtggtttctctgattctttgagtccttttgaaataatgtaatatttaaacagtaactgttttacaatacaattgacgctgagttcaaagggggattagtgccggcnaaggcagagagacaaaggccaagtgcagtccagggctttacttgccaagtttccctaggtgaagttccccgaggtatgagttcttgtaaataagtaacagagtcctgagaagcc

SEQ ID NO: 114 - DNA, 284 bp, rat
  misc_feature (1)..(284): Clone #119 (MLSSH)
gcctctagatgcatgctcgadbggcgcaggtgatgatatctgcadattcggcttcgagcggcgccgggcagtacttgtgggatacggggggatggagwgatcagagatctagctacagaggctacttgtagacttctccctcgatacctgctctgtgtctagctggyatggctctgcwghgwgwttgtgccacactgaaagttgggactcatgavgcctgctcccatgagcattcttctgttctaacagtttacgaggagacagatggtttgtatagtcatcag

SEQ ID NO: 115 - DNA, 278 bp, rat
  misc_feature (1)..(278): Clone #120 (MLSSH)
gtacagccggaccgtggatcatgagtgtgggttctgatgtcctacacccagtaggagtgtgtggggacactcttcactcctctggtgcgcctggtagaaaggtctgcactattggggattgatttgatttgatttcctaagactccaactgaaacaggacacgttaacttagggtaatgagtaagtctggggaggtgaggagccaaggagtgggtgggcagagactctcttgggtcttttcttactggcacacttggatgttcacgtgacatccgccc

SEQ ID NO: 116 - DNA, 195 bp, rat
  misc_feature (1)..(195): Clone #121 (MLSSH)
gaggthhavbbytkwwtttttacactgtcacctattadhyttgagaaatgactttttatagtaagtgtatttggtaattctggctcctgatcccaagcctggaaaggcgctgcagccttgtatctctaggaaacagactcaggtcacagagshbccacattctctgtggcattgtaagtctaaggttatactcag

SEQ ID NO: 117 - DNA, 414 bp, rat
  misc_feature (1)..(414): Clone #122 (MLSSH) n is c, g, a or t
acttaagaacttactatcagagtgaccaaattataccaaagaacgcaaaagagagcactttcaaaatattttcttgccttgtnnnntttctaaaattctgtccgaaagagaaaagctgagatacttatgttcacttggaatgacataaatatcacccagtttgacattccaaaacactctgtgcagggctagggatatggtanacagttgtcgttcacaatgccccgggttccatctccactatctcagctaaaaatgaaagctagatttgtttaaagaaagttttacttctccctagagacttctcattttgctttgttcagtataccacaaattcaacattgtgctttttgcaggaactgaggccttcagtcctttttgtattagtaactgtcaagaagcctgtttacctta

SEQ ID NO: 118 - DNA, 179 bp, rat
  misc_feature (1)..(179): Clone #123 (MLSSH)
acagakagggttagaacagaacccagaacttctacagaatagaaaacccacccttgcctatttgcatggarataccacccacagagctaataagtcttacagttatccggaggcctatgtgctgtttttaaactcaggactctaattttgattgsmmcatagatgattgataaaagtca

SEQ ID NO: 119 - DNA, 238 bp, rat
  misc_feature (1)..(238): Clone #124 (MLSSH) n is c, g, a or t
gggcctctagatgcvgctcgatnycgcagttgatggataccgcagathcggchagcgtggcgckgccgaggacaggwwaggtccgggcacaggagcmagcacmccttcacaaagkdacccaagggagcaggcagcgcgagacaggcttcctaccgagayhgggagaggagagakggacgtygchcaggagtcccagtgagagagagtaccttagacaggggaagmagatccdgggcag

SEQ ID NO: 120 - DNA, 377 bp, rat
  misc_feature (1)..(377): Clone #125 (MLSSH)
acactgcttccagcaaattgcggtgtatgtgggattttctgtaaaatgggtgtaatttttccctaaagaaggtgtgtgacaacaagagaggttgcctgcatgccagtttaaattgtttatgtaaaaatgctgttataagacatagatttatcctagttccttaatttttttccttatatgaaactttaaagttctttataaaatacagcagtggaaaactaacaagttgaacgtaaggttggaaatgttcagttttatgtgtgcagacacataagctatggggtaacaatcaaaacgtaatcttttgtagatagccattacatttctttaaactatttcaatgccaatgtatattctataagagaatgggttcctaa

SEQ ID NO: 121 - DNA, 223 bp, rat
  misc_feature (1)..(223): Clone #126 (MLSSH)
aatctttacggggtaagggttctttctagtttctatatgaagtatgactttcttgagactgtggcagaactcagatgttggtgtctttacagggaacgtgcttaccactcatcaaattattcatgggaaaattaaaggtggtttctggtaactgggataaacagagaccaccaaacttaacaattatcttaagggggcaccccctaatccaabcccagccaca

SEQ ID NO: 122 - DNA, 643 bp, rat
  misc_feature (1)..(643): Clone #127 (MLSSH) n is c, g, a or t
actctcgggcctgtagacagggaatagaatagaagttcctgggctctcccagaggaagctttggaggccacaagcctttgaggaagctaccaactccttacaggcccctagcctgagcaggtctgtacccacaagccccaccccctctgaaggctgctgggaggcgcggcagtcccacacacaggtactagctacacttcatcccagctcttcagaactcacccctaccagttgttcttgctgccacctccacctctggaaagccagggacatgttcctggaaattccttattgavvgtccagaccgacatccacaaacaagaagataacaaggacaggtgnnccagctaactcccaagatttggaaaccacagttccaactcaggttcctaagaacctgtctcctgaggaakagaagccacagccaccaagtgtccgtgggctgacccatccgcttcttgaggtccccagccagaactggttatgggagtctggatgtgagcaaatggagaaagaggacccatccctgagcatcaccgaagtgcctggtttctcaggcaacaggaggcaggacattctatgcagagcccaccatctgagcccagaaacccggcccagcctgctctggagaagccaagaactt

SEQ ID NO: 123 - DNA, 194 bp, rat
  misc_feature (1)..(194): Clone #128 (MLSSH)
acttaaaagtggaatgctcaggtaaatacgattaadctcaaaatggaaggtgggaaagacattctgcttccatggacgccagaatcaagaatacttcccctacgacgcaaactgttaactttagatagcctgttggwactttataaaatagttacyttawactggccaatagrgatcatgttttattgtaaaaa

SEQ ID NO: 124 - DNA, 401 bp, rat
  misc_feature (1)..(401): Clone #129 (MLSSH)
acaactgtcccctggataaaggtgggctcactcagcttaacagaagcaacaacgcagagataactagatggcactcagtccagcaatgcaggaggggcaaaaagcagcttgtagagtgatcgatccatctattcccagattcccagccagcatgagaccaggcttcctgtaggacttcctctcacagatgatttggaaaacgtttcacttctctggagtttatcttttcttctcactgaagggatgagtgaagctcacttggagtaggctgcaggacagtggtccactcttccgtccacggtttgagtttattttaggaattcagatataggtcagaagacatcacttcatgggaggaccatgaccggccactcggcagaaacagcttaggccttgtttgt

SEQ ID NO: 125 - DNA, 326 bp, rat
  misc_feature (1)..(326): Clone #130 (MLSSH)
accccatgaatattcagacaggacacaagctgcaagttcgctggagacagtccctgtggggtttttaactacttcattacaagtatgaaacatacacaggccatctgtttaaagaaatatccagtcgctatgttcacaagagaatatacaggtctagcttgtagcttcaaaactaggtatcaaaactttaggccagggtatggaggagcaatcccttacttctagcacgggtgargcagaggcagtgggatctctgagatcaaggccagcctgatctacaaagtgagtttcaggacagccagagcaatttagtgagaccctttctt

SEQ ID NO: 126 - DNA, 563 bp, rat
  misc_feature (1)..(563): Clone #131 (MLSSH)
acactttctcattgacaactcccacggtgggaagacagtttattacttagtcttactttttttggacagctcattcctgcacaagtgagagacatttgaagagtaagtctgtttgcgatctgtcatatttgaacccttctacaaaggagagctccctaaattgaacttcccgaaatctaactttcctcaatttccttcctaagacttaaaaacatcagtaattgagggcatctcctgattaaaagtcccctagaaacagagccaccaacagctggggccacctgaacaagagtggaaggttccaaacagaatactgttcaatttcatgcaaactctgactcacgaatgaacagttctccctcccagtctgaggagagaagtctactcttatcctgacagcaattagctggctctccttaagaaacagtgatcaaaggcaacatgagcttatgaaatgttcagttacaccatgagtcacatgagctggcgaccagactcagtgacaatggaactcagactcacgaatgagtgaacctaattcctcaacaaataaattctgaaaa

SEQ ID NO: 127 - DNA, 233 bp, rat
  misc_feature (1)..(233): Clone #132 (MLSSH)
actggactcacccatccacacgtcaccttagtgtcacttattggcaacgccatagaacagcaggcccttgcattctacatttcttttggaagttacccattcatgagtaaaaacaggaaaatgtcacttggtaaatgcaatctttttgttttaaaacctagaagacgggtgaatgggaaggggagtggttctgatactkgttgtctggaacaaagctttaatgaagatatgtt

SEQ ID NO: 128 - DNA, 354 bp, rat
  misc_feature (1)..(354): Clone #133 (MLSSH)
accctacaaaatgkagactctaaaggacccactaatgtcctgactgctgcagtttcccatcakaagaccagactcaaacaagagactcttccaggccttttctttatatggagtcttgctatgtgacccgaactcaagctcaggctcaagtgatcctcttgccttagtctccccatgtgctgggcttggattattagggtgcccttaakataattgttaagtttggtggtctctgtttatccagttaccagcaaccaacttttatttttcccawgaataattttaagagtggtaagcacgttccctgtaaagacacacaacacactgagttctgccacagtctcaagaaaggca

SEQ ID NO: 129 - DNA, 413 bp, rat
  misc_feature (1)..(413): Clone #134 (MLSSH)
accagtgtttacctcaggccagcatgcctatgcttttaaaggttttagttccttgacagttgaggcgttagagagggaaaagggtatcaatacacacaagcattcactttggttagttatattgctttctggaatacactgttcaaataggcttatttttgtgacattaaaaactatgggatttctctttattaaagtccaaaccattaacaatgacaaaagcacaatattaaaataataacacgggaagccaagcagtcagctacagctcaaactaggaactaagactacaaggagctgcttcatttttggtttcaggttttggcccctacttcattcttatttacagcggatacctgcagagaaaaacacgcagcaaggccttttacaaggcgcggtttgtaaggcgcacc

SEQ ID NO: 130 - DNA, 408 bp, rat
  misc_feature (1)..(408): Clone #135 (MLSSH)
acatgtgaattgattatgatctaatacaatcccaaacaaaagtttagagacatttaaattttccctccacgaaaacagatgagatccccaaactgtacagagagttcgagactgcccagagttcaggccagctgagcagcactaacgggtcctcaggcttgggcacaccacctgtcaagtacacataaagcacaacttcccgatgggagggcacccgaccacagtgcttcaagtaaacacccagactttagagtatgataacagccatcactgcaaaaatacatccaaaatgaataaaatgtgtgaggaaagtggtgcttgtatcacttcaaaccaacccttaaacatttcaagtcactttctaaccggcattgtgtgtgcagttgttattcaaagtaaaaaaaaaaa

SEQ ID NO: 131 - DNA, 205 bp, rat
  misc_feature (1)..(205): Clone #137 (MLSSH)
acaagcttttttttttttttttttttttttttttttccttgtatgaactttttaatgaattatgagccaatgaacattatcagacattaagccttaatttttcttctttcctatgcaaacttaaataatataatcgcccgbtttcaagtttggctatgaagaccgatagatgatataagagttagttactccaagtttgaatcca

SEQ ID NO: 132 - DNA, 134 bp, rat
  misc_feature (1)..(134): Clone #138 (MLSSH) n is c, g, a or t
acaagcttttttttttttttttttttttttttttttccttgtatgaactttttaatgaattatgagccaatgaacattacagaattaagccaaaattttacttgctttnccttatggcaaaacttaagatacta

SEQ ID NO: 133 - DNA, 185 bp, rat
  misc_feature (1)..(185): Clone #139 (MLSSH)
gcatgtgatgtytkcratcgctacgtgcscgcgagttttttttttttttgagagaatgattatggtgtttaagcatgctgcaaggggggtctcagagtacaggctaccacatcacaacatgcattcctcacaggacatgtgcagaggctcggggaycgtaagtagtcgggargtgggactccagt

SEQ ID NO: 134 - DNA, 216 bp, rat
  misc_feature (1)..(216): Clone #140 (MLSSH)
actgcggacttaaataaaggtttttttaaggtattgggtcagttgacagagactggtgtcgtcagcccggagcagttcatgaaatcttttgagcatatgaagaagtctggggattactatgttacagttgtggaagatgtgaccctaggacaaattgtggctacagcaactctgatcatagaacmtaaatttatccattcatgtgctaagagaggg

SEQ ID NO: 135 - DNA, 208 bp, rat
  misc_feature (1)..(208): Clone #141 (MLSSH)
acaagctttttttttttttttttttttttttttcaaattggttaaataaactccatactttccttttaaaatcttaatattaaaatattattatctcagtaagctactctattaagtttagagttctagtttccttttaaatctttttaaatagaaacttatctgcgaatgcctcaggtgagtctcctgcctcaccaggcgactgccg

SEQ ID NO: 136 - DNA, 527 bp, rat
  misc_feature (1)..(527): Clone #142 (MLSSH) n is c, g, a or t
acacttgtcaaaacagaaagatgggctgtccggaaaatgagatgccaattctgtgaacaatgggagtttttttgtgtttgcttatttttagtttttttttaatattttttatattttttttctaaaaagaacagctgaaaaaccctttcaacgacatgctaactggttgtcaccctttgatatggtcacactatggtcaggatgggtgctaagggctcggaatagatgcacattataaaattcttgaaaaagggatgttccttgtggcttttttcttggttaagtgcctaggggcaggagaggggttaacaaaacactgctcgatgatgaattttcttttacacaccagtccgttcccaaaaggccctcatattgcangtggttctatcagaagttaaaatgaagacaagaaaaagaaatatcctttcaaataacagagtcgacacttttttccccytaaataaatcagcataagttttccacataatgtaacaagagtaaaaatgcaccttgcaaaatccaaaagt

SEQ ID NO: 137 - DNA, 269 bp, rat
  misc_feature (1)..(269): Clone #143 (MLSSH)
gattggcctctagatgcatgctcgagcggcgcagtgtgatgrtatctgcagaattcggcttcgagcggcgccgggcaggtacagcttttttttttttttttttttttttttaattaaaggccacttataaacacattcttcttcagaatctgattttaaaaacctttatctactaaaaacagacatataatcaggcaacagttaagactacaggaatggggctctagtgacgctgaaggaggcgataggtggggaagggcaragggctg

SEQ ID NO: 138 - DNA, 198 bp, rat
  misc_feature (1)..(198): Clone #144 (MLSSH)
aagcdttttttttttttttttttttttttcgatgccaatcaaaactttattattgttttaaaagatacaaaatagtcaaaaaagttacatcagctatatatgatcgccctttcaaaatccctaaagagctgtatggcattattagctgrgagtaaaatactgctactaattcttagaacaggattttcagagcctcaa

SEQ ID NO: 139 - DNA, 137 bp, rat
  misc_feature (1)..(137): Clone #145 (MLSSH)
acaagcbttttttttttttttttttttttttttttgagcaatatcattgaagcatatatttvrtgattattattatcatcatcatcatcatcatcattattattttcacattttaaatattacatttccttctttcc

SEQ ID NO: 140 - DNA, 188 bp, rat
  misc_feature (1)..(188): Clone #146 (MLSSH)
acttgggctttgggctcctcacctgtgaaatttttttcytttttcyttcttcctttttttccttttttttttttttgtgggktttaacctaagaccctgtcactggacaatttybataggttctgctggcatcagcaaatttcagccagtgcaccccccgggtttgcccttggtggtcctcgtactag

SEQ ID NO: 141 - DNA, 553 bp, rat
  misc_feature (1)..(553): Clone #147 (MLSSH)
tactacaagcgaaaggaaatcttcaagttcagaaacacgtcattttcaagggacagtgcggttkaattagtctgccgtcatgaggcctcagaagcaccttcatttaactgttctgaactttggtaggtaacgccttactgtgggtggtcattgtttgcaggatttgctgcatccataaagtagtcattacattgctttaagttttgttatgatttatattacagtttcctggccatttttcaatccacagtagtcacaattctaatgaactatagtagttctaataccttgctggagacagtagaaacggtatctccccattcccttattagtggacgtggaaattgaaaatcagtttaaaatagtgaagctttgtaaagtgcaaggtaaaactaaaatttagggctttacagtattcagccctagactattattccatgactattttataacttaatttgtttttgcaattgtataaataagtcattgagtctcagtcccaagtctgtagtgtgttgctctaatgagttgatctctgtgaacgtgggcctgt

SEQ ID NO: 142 - DNA, 137 bp, rat
  misc_feature (1)..(137): Clone #148 (MLSSH) n is c, g, a or t
tctggagagctgaagtctgagcgctgtatatttggactcctaagattgaagatccggcatcagagaaactgtcnaaatactggaaggtgaaataaagacttwtcctttcaaaaaaaaaaaaaaaaaaaaaaaaaaaa

SEQ ID NO: 143 - DNA, 202 bp, rat
  misc_feature (1)..(202): Clone #150 (MLSSH)
ggaacgtctaggcaacgtggtctccccgcccgcggdtaggcaaaggcgtttgcgcttcccagcgtctgaggcctagggagccttcagtagccgaaagttagtcttttgcagtggagtaagggctcgcggttagccgcgtagccccgggdwctggcctcaccatgttggtcctatttgadacgtccgkbggctacgccatctt

SEQ ID NO: 144 - DNA, 155 bp, rat
  misc_feature (1)..(155): Clone #151 (MLSSH)
acatcgctgcataaaatgcaatctaaatgtcctvttgattttatgaatgbbwgggcttgtgctttatgaatctcaagtttttagacgacagtagtgggagarbcattaaagataatgaacyggtttgtgtggtttttttttttagaaaataaatt

SEQ ID NO: 145 - DNA, 215 bp, rat
  misc_feature (1)..(215): Clone #152 (MLSSH)
acatctagaagcattactgtagaatctgagcagattatgtgtaaccaaaataagaaagagtataagaaataatcctgaagctaggtatgtctcacaattttgtagaatcttacagcatatttgataaatttctcagtgaaaatgttggctaggcaagttcagttaaaatatagtagaaatgtttatcctggtttctctaagtatacattaattgt

SEQ ID NO: 146 - DNA, 705 bp, rat
  misc_feature (1)..(705): Clone #153 (MLSSH)
acacatgtaaattaaaaactammmgttacaaattttcaagatcttctctagtatgaatcttgtaacccatagaacattatttccttttcaattctagacctattttaacatttactcatatggccttttaaatgttggtatataagaactttctacattataattatacaatttttcaaatttcagagttattatcaaaacctgaagtatttgcttgaatattctcatgggtgtcaaaccttcacttagtgaagataaataatttttttttcaaattttcaattctggtcaatattaaatgtgattgaactgggattccattgcagaccagatcaaaagattatggttataaaaacccatgttataaatctcatatcaatttgatggtaatcataaaagaaacgtttcaaaatgtccttagggtatcataccattctcagaaaaagaagtcatagctcagtaatagccacaaaacattgccactaatgtgactatgaacattgtatcttattagtttccaaggtcagtctcaaggctttatactactatttgtaatttaaacacttggatatattgattagtcaaaactttaacaataaagtattagtttaccttttttaaatagttctgacagatttaaaagaccaatatgtataattatatgtaaaaacctttaattgctaatatatatatgtatatgacttc

SEQ ID NO: 147 - DNA, 283 bp, rat
  misc_feature (1)..(283): Clone #154 (MLSSH)
aatgggctctagatgcatgswhgaagagcgcagtrtgatggatatctgcagaattcggctttcgagcggcgccgggcaggtctcagaattctcagaaagccaaagatttctttttttactgccttcagcagtgagcacaaagctcatttcaaaggagactgacaaattgtcccagctgcagawggagaatccttcctggacgcttcctgtctccagtgagttgyaccgacctgctgcaaaatgaactcatcagttcctaggaggtgtggcttcttcacctcat

SEQ ID NO: 148 - DNA, 350 bp, rat
  misc_feature (1)..(350): Clone #155 (MLSSH)
taccctgccccctagggttggttccacctgttttttagacaggctctccctaatggcgctctgactgacctccagctctcgattttatttttgattgtttgtttttttttcctgtctcaccctgagtccgaactccgcctatcgctccgttactggcatttctcgcttttacctactgtagctggagggattttaccctgagttcctgtgtctttattcattctggggatctactgtgcaaagtgttttaaggctgcctgtgaatctggcctttgctattctcaaaatggcttgtcccgagatttcctgctgtggctggaggtaactcacaggacagacacgcccgctcc

SEQ ID NO: 149 - DNA, 592 bp, rat
  misc_feature (1)..(592): Clone #156 (MLSSH)
accagaagcctgcagagaatggaagtagtatctttagtgtcctttcctggcatgtccatcttgtgtcactgcatagcaactggagttttgctcggatctggttataaattcttgtacttactggagataaacatgaaacagctgttagtgttagtttgtcctgtggacattttcatagaactatgaatatcctggaacttatcaaccagaagtcagacagtggatgtgctgaacagttgagacagcttgccaggagaattacagaggaccatgtgatccagcatgggctggtagtagacgggaccagcttatctctggcactcagggagcatgaaaagctatttatggaagtttgcagaaactgttcggctgtattatgctgtcgcatggcacctttacagaaagcaaagtgataagactgataaaaatctcccctgagaaaccgataactctggctgttggagatggtgctaatgacgtaagcatgatacaagaagcacatgttggcataggaatcatgggtaaagaaggaaggcaagctgcaagaaacagtgactatgcagtagctagatttaaatttctcgccaaatta

SEQ ID NO: 150 - DNA, 372 bp, rat
  misc_feature (1)..(372): Clone #157 (MLSSH)
accagttgagataaccatcagtttattaaagagggaaatggaccaaacaatggctgccaatgctcagaagaataaattcttgattgatggctttccaagaaatcaagacaaccttcagggttggaacaagaccatggatgggaaagcagatgtatcttttgttctgttttttgactgtaataatgagatctgtattgaccgatgtcttgaaaggggaaaaagtagtggtaggagtgatgacaacagagagagcttggaaaagagaattcagacttatcttgaatcaacaaaaccaattattgacctatatgaagaaatgggcaaagtcaagaaaatagacgcttctaagtctgttgatgaagtttttggtga

SEQ ID NO: 151 - DNA, 529 bp, rat
  misc_feature (1)..(529): Clone #158 (MLSSH)
acaaagcatacataatgtgctttaagcatcctttcatctttccttagcgcccgtcctgtattctccatgtgagtgaaagttgtaaccatccatgttctggacgtggcatccctcaggcattgctgttagtcatccttcakagcgtcccctgcagtcattcactttccagtcaagctggtctttgaaattccctttttctgtgcttgctgctcacctccattcttgaagctgcacagttgtctggttgtgcttacatttttccaatcaatctcatttcattatctgcattctctcatttttatctgagccatctcttttgtttcatttgtttgttttgtctagaaatatctaatcctattacttaaacattactgctgtctttagtttggctactcaacctgtgtctatctgaccaagttactgtatagtttctcatttactcagttaattttcatattttakagactttctatgtaaaaaagcaaaatgtcaataaacctcttttatgagttccaaaaaaaaaaaaaaa

SEQ ID NO: 152 - DNA, 577 bp, rat
  misc_feature (1)..(577): Clone #159 (MLSSH)
accaagactgcagcaaacaaaacaagttaaacttttgaggtcctcattgcctcaaggccctgacctgttattttgcacaggaagcagagcagttggttacaagccaatggggggaagtgtcgaatttccaccagcccttgcttaaaactagtttaactccacagtccatttcctagcatctctgtgtttctactctctgcattatcgtaatctgagcagtccacctttttaccatatcagttggatctgatacagcaagtagaatatgattcttatttataaaaaaaattcttggtagtttgtgttgttttaaaaaccagaagcagattacaaaagttacacttattacatttttaactgtaacataggtgtgtgctcayttgggacattttttggcttatttctacactttcaaaaattgctataattcactttgtccctctatgatagaaatactatattagtatttttttaaccaaacgcctcctttgtttaaacctggcccygagtttgacctgagtataaactttagaacttacaatgccacagagaatgtggctgctctggtgacctgagt

SEQ ID NO: 153 - DNA, 619 bp, rat
  misc_feature (1)..(619): Clone #160 (MLSSH)
actaagctttcttagaaatgaaaaagttccaaatattagtgatggtaaaaatatagattagggcttgttaactgggaagggagaaaattacagaagtaatctacttttcagtagattacaaatatatttgcttcaacctgtaacagagcctatcactgtttggcttatcaggcagacagaaaataaaacctcaataatactcctgataagatcttggtctaattacacactgtatcctccaacacacctcactgatgccagcacaaaaactttgatcttccattattcaagaataaagagatcatctcacaagaggctgtggtctgcagctgaaatccctatccaaatatcacattcctcattctgttctcacaatatgcaatgaactttataagattctgccccagtgtttatgattttaagaaagctgcttcaaaatattatataatattaataactttctgtaatttttctattcagttccttatgattcttgatagaatcacacattttcataaaggttgatctctaatatgcatttaataatgtttgtctatcactactaaaataagaatgaattaggtattcatatttacttcgattccataaaaaaatcaca

SEQ ID NO: 154 - DNA, 191 bp, rat
  misc_feature (1)..(191): Clone #161 (MLSSH)
acctcatgaaggctgacatgatgccgttcctgtattggaatatgatgctaagaggctactggggaggtccagccttcttgcgaaagcggtttcatctgggcctgagttaaagatggctcagcacttgctctttttggatgatgtctggaccaacatcacagcttactgattgacatgacagatccacagga

SEQ ID NO: 155 - DNA, 187 bp, rat
  misc_feature (1)..(187): Clone #162 (MLSSH)
gaggtaagctttttttttttttttttttttttttcgatgccaatcaaaactttattatkgttttaaagatacaaaaatagttaaaaaaagttacaatcrcctatatatgaatcgccctttccaaaattcccctaaargrrctgwatgcatttatttrcgtgrgdtaaraatcktcatactaattctt

SEQ ID NO: 156 - DNA, 206 bp, rat
  misc_feature (1)..(206): Clone #163 (MLSSH)
cgattggcctctagatgcatgcygsgcgcagtgtgatgatactgcratcgcttcgagcgcgccggcagctttttttttttttttttttagggytagccagccagcagcgtcacgcattattkkkkvacatagaagtagcatcgagcttcctawwgagaagtgaaragagatctagtcamwggtttgggcattaaaacaaatcaaaa

SEQ ID NO: 157 - DNA, 250 bp, rat
  misc_feature (1)..(250): Clone #164 (MLSSH)
gattggtcccwgakcakcvgagtgtcgcaggtgatgawvttcakaatyggcttcgagcggccgccggcggacaagcvttttttttttttttttttttttttttaattaaaggccacgttwkwwhacatcttcttyagaaccgattwaaaaccttwyyhcswrrrhdgcaatwactacaacagtwgmcaggaagggccattghccgwagagcraaggggaagcaaggccttggagggagcatgaatgccca

SEQ ID NO: 158 - DNA, 512 bp, rat
  misc_feature (1)..(512): Clone #165 (MLSSH)
acaagctttttttttttttttttttttttttttttctgaatagagaacaaattttattctaaaaatagatcttagtaacaatgaaataccaaaattggtcattacaatagaaaaatggcaagtctttctcctgaagatacaaaattaccacaccataaaatatgttgccaactttcatatttgaattagtccttttatggatttactcactgttttatcttaaaaaaaacaactcattctctagaaaagctaggaaagtaagcatttgcttaagctagacttggaagacaatttaggcttaggaaacacgttcaatttttacagattaaaaagaagtctcttcaacagaaatcctggtatttgaaagtaacaaattagaaagacattacgtaaattacttttaaccccaactcggtggaaaagtaagaagcaaataatgaccttgctttaagaaagttgtttgtatttccaaagtgctagagaaataagtattcacgacaatcttggagggt

SEQ ID NO: 159 - DNA, 63 bp, rat
  misc_feature (1)..(63): Clone #166 (MLSSH)
acctacctttgtttatttgctattaaactgtttgagaagaacaataaaaaaaaaaaaaaaaaa

SEQ ID NO: 160 - DNA, 252 bp, rat
  misc_feature (1)..(252): Clone #167 (MLSSH)
ttgggccctctagtatgcatgctcgabcbtcgccagtgtgatggatatctgcagaattcggctttcgagcggcgccgggcaggtacaagcttttttttttttttttttttttcccttttttttttttttttttttttttttttytttttttttttttttcaagaaacaaatcttatgacacttctgtgcagavagaacacatagagggcagrgcaagacctgtcgkgagttgttcacagtctcctaaggaga

SEQ ID NO: 161 - DNA, 296 bp, rat
  misc_feature (1)..(296): Clone #1  (PLSSH)
acacaggattacatggaggcagcggtctggcatgaagggtgtggacggacagaaggttctgctttttgctagtcagacaggatgcgaacaaaggacactggaaagacacccttcctttcagactgtccttcgatatgcccaatccaccactcctggtcctcttccccagtgacaatgatcacttcaccctcaataaatgtgagctcatcatcattgtctgcctggcagtcacaaatggttttcacacgcctcactttatttttccccgtattgatttttctgggcagtggtacctg

SEQ ID NO: 162 - DNA, 187 bp, rat
  misc_feature (1)..(187): Clone #2  (PLSSH) n is c, g, a or t
acagaagcagatgcatagttacagggattggtggattcaaacgaggcacatagacatgttcacatatggttggctatttcacatgatgaggtaatanggtatagctacacacattggcaggttcggggcgtcctggatgtcgggggctsggggcttatctcttcctgccaggtggcctgtgagtbgt

SEQ ID NO: 163 - DNA, 131 bp, rat
  misc_feature (1)..(131): Clone #3  (PLSSH)
accttcgcgtgtctcttccaaatatgggagaccctgcacacctggcaactacggcgaatttgtccaagttcctggctacccatttcctgcctatgtcgtctattcaggtcagcacacgatgactgcatggt

SEQ ID NO: 164 - DNA, 389 bp, rat
  misc_feature (1)..(389): Clone #4  (PLSSH)
ggcgattgggcctctmvatgcatgctcgakckgccgccagtgbtgatggatatctgcacsaacwtcggctttcgatrcgtgccgcccgcgtgcaggtadctctgaaaarggggacgtcactctggcaatgaadtgtcactttgccagctgtgttcaaagctcccatatcgaycggatattgtbamcyytgtttcacaccaaccttgbagcgvagcarcaatagyagcagacccctaatgtctcgttcaggtgtaatytagycagsgabtcaktcaggaccsacattgcmtgcagttccctbgtgvgataccgcccccmtcycgyccccacgacymcttagagbcccssaaatttcccagaccatcacctkggcccsgcbcbmttthtvt

SEQ ID NO: 165 - DNA, 90 bp, rat
  misc_feature (1)..(90): Clone #5  (PLSSH) n is c, g, a or t
acaagtgtagggcgcatcttctcctctgtcagcatgcaactgagccatgactgancactcagaccccaggtccaaatatttggatactgt

SEQ ID NO: 166 - DNA, 350 bp, rat
  misc_feature (1)..(350): Clone #6  (PLSSH)
acaacatgtatatagctgctgttacagaccaagactcatggaagggccactcaggaggggaatatagtatgtattgtaaatatcctgtggccttctgccttcaccagtattagggtaacctcgggacctggtgcgccttactggtttgccaaagccatccttggcatctagcacttacatctctctgtgctgttttatgagcaaacaaaaaccaggagtataggaactgctggctttgcaggtgaagagtctccagcagctgtcaaagggtgcggaactgacttctaacaatgcagtattcccagtggcgttactgaacatgcaacattagcagaggtgggttctgtgct

SEQ ID NO: 167 - DNA, 169 bp, rat
  misc_feature (1)..(169): Clone #7  (PLSSH)
agatatacaagattccctcacagtatctaatgaggtgcgaacagcaaaagaatttataaagattgtagaagctgcagaaaatgaataccagactgccatcagtgagaactatcagacaatgtcggacactaccttcaaagccttacgtcgacagttgccagtgacacbc

SEQ ID NO: 168 - DNA, 206 bp, rat
  misc_feature (1)..(206): Clone #8  (PLSSH)
gtacttgggggtaatcaaagacctggtgctggtggcagcgctttcggacggwwaggacgccctctgagagaktggtgcatttgtvmaggactatayayggactctctgatgtttggghctgggagggctccctagwttccctctcatgtracgcchcttctcgaaacyaygacyctaaagcsygaattyccagkcacactgggcgg

SEQ ID NO: 169 - DNA, 102 bp, rat
  misc_feature (1)..(102): Clone #9  (PLSSH)
gagcaccatgtgttgctgatgaactgagttcaatcccagggacccacatggtgagaggttagaatggacttgcaagtttaagcaacactgtctgtttctgta

SEQ ID NO: 170 - DNA, 125 bp, rat
  misc_feature (1)..(125): Clone #10  (PLSSH)
actaaagaaaggtttgtattcaaccacgtagtaatatttcaagctactaaaggaatagtttacctatttagacagcagcaatttctactacattttttataagcttaaaacacactatgtataca

SEQ ID NO: 171 - DNA, 120 bp, rat
  misc_feature (1)..(120): Clone #11  (PLSSH)
atattagtgcagagttgtgcagtgggtctgcagggcagatacattagtgcagaggtttgcagtgagtctgtagggcaggtatattagtgcagaggtgtgcagtgcatctgtagtgcagga

SEQ ID NO: 172 - DNA, 295 bp, rat
  misc_feature (1)..(295): Clone #13  (PLSSH)
acaaagggcaatcctgcacgatggagggacttccactcagccacaatgctgggcaatcacatgtatgtcttcggggggccgtgctgatcgcttttgggccatttcattccaacaatgagatctactgcaatcgaattcgagtctttgataccagaaccgaggcctggctcgactgtcctcacacgccggtgctgcmtgaggggcgcagaagccattcagcctttggctacaatggagagctgtatatctttggaggttataatgcacggcygaatcgccatttccatgacctctg

SEQ ID NO: 173 - DNA, 270 bp, rat
  misc_feature (1)..(270): Clone #14  (PLSSH) n is c, g, a or t
acgattaaatcaccgtgtctagctttgtctacagacttcacggggcctttcagcgacacagcgtagagaacaggccactgggtaggctcttytnaaatcatttgcacctactgaaatctggaagcatgaatccagccattgcctccagcacttaggaggcgtctgctgagatgacttgtctagatttttgaaatcagtacattcccaggcatgattttctcttgatagctgaagacccttggatgtgtgacttaycaagctgtgggtact

SEQ ID NO: 174 - DNA, 88 bp, rat
  misc_feature (1)..(88): Clone #15  (PLSSH)
caaaggagcccttatttattatgtgcagggccctaggttcaacctccatacccccaaagaccaaaaaaattaaacggatgtgtaatgt

SEQ ID NO: 175 - DNA, 173 bp, rat
  misc_feature (1)..(173): Clone #16  (PLSSH) n is c, g, a or t
cctyctngtctgttctnnnttcctttccttttatggtctgttagttttcacggtgactcggccctaggtatcnbggcgtgcctgggctcgttcaagcctgttgcaaccctgagttaagactcatggggtgggtctttnattttcccccattttcttttagacttgagtaaaat

SEQ ID NO: 176 - DNA, 227 bp, rat
  misc_feature (1)..(227): Clone #17 (PLSSH)
acatcttatggcgagtggccacccctgagatattacctcattatgcagaagtaacatacccttcatgactgttttgacaaaagcttaaaaccacatctgatggaagtccaactttcaggaaccaaggactgccagaaaatatctgcctctacgttatgcatgcatctagacgcttacctgaattctgccttttataaaggracaaacagtatgaagaagtgaactgt

SEQ ID NO: 177 - DNA, 99 bp, rat
  misc_feature (1)..(99): Clone #19  (PLSSH)
acaaatgtccactggggatgctgtgcgctccaggaatcgtctcgatgtgagggtccaccagccggtgacccggatagamatgctcatccacaggtgtgt

SEQ ID NO: 178 - DNA, 260 bp, rat
  misc_feature (1)..(260): Clone #20  (PLSSH) n is c, g, a or t
acggwatccctctctgccaagccatcctggatgaaaccaagggagactatgaaaaaaatcctggtggctcctgtgtggaggaaactaaacatcccaactgctctgtaagattccgaggadkvcatctcttagccgttgttttcttcctabtgcraggcttnagtagghaagttvgctttgtcagtaagtctymattgaccttbctttgaataatggtagcccataaattatggttttagatcattcatctgtacctgccc

SEQ ID NO: 179 - DNA, 981 bp, rat
  misc_feature (1)..(981): Clone #21  (PLSSH) n is c, g, a or t
acaccaacattattctaataaacgccctaacatcaattgtattcyaggcccttatatcatatcaattaccctgaattgtactcaaccagcttcataacagaaacactacttctatccacaactttcctatgaatccgagcatcctacccccgttttcgatatgaccaactaatgcacctcctatgaaaaatttcctcccactaacactagcattctgcatatgatacatttccctgccaattttcctagcaggaattccaccctacacataacagagaaaarncccnnnnggggnncaaaagggnaacaaaaaacctgccccggggggggcgcttccccagccgggcttcttttcgcgaacccatccccccccgggggcccgcccccrgcaaggcccytcgggggggcccaattcggccccwaaggggagtccggatttaaatttcccctggccggcggcttttttaaagggggggaactggggaaacccccggsggtttccccacttaattggccttgcgggaaattcccccctttggcccaaatgggttatttmgccmaggggcgcccccgcttgggcttttccaccaatttgggggccccaaagggggaatgtaggggccctttttrgggccsttttttcccgggggggtcttggggttaaangagcsccgggctaaaccttttagggccctagggggggtccctttcctttttttccctcccttttgccccctttccggggttttcccyagaggttaawtttgggggccytttaggggtcccctttggccctttgggccctgtttccccnaaaattttttggggggggntccaggtgggmccccccccttaaggcgnttttccccctcgccggggntnccccccttttaaaggggactcttttcaagggggaaaaatttgcccccgggggaaactcttttttttagggagttkgccctttgcccctgkttnaaaaaa

SEQ ID NO: 180 - DNA, 351 bp, rat
  misc_feature (1)..(351): Clone #22  (PLSSH) n is c, g, a or t
ggcggacktgcgtgccgtctaratgcatgctcgagckkctgccagtrtgcatgcgatatckgcagamktcggcttatggctgtgagtgcggaccgngctgtgtacccaaggayttggcggctttatttagtagcaytggcgcgctcgtctnnnnnnnnntacgaacgtcgaagaatgaagtctcttgctactaccttgaaacatttgttcaacctctctttcdgtttgattctcaactactaccattaaccctgctgcaagatttgtcaccgttctgcctgattgctgagactccgttttgctgctgtgcaagaagaaagaggcaggcctaagggagcatgcgtctcagca

SEQ ID NO: 181 - DNA, 447 bp, rat
  misc_feature (1)..(447): Clone #23  (PLSSH) n is c, g, a or t
agagattagaagcagcattaagaggccttctgggagccttgacnnscacaccatactcccccacccagcatctagagcgggagcaggctctggctaaacggtttgcagagattcttcacttcacactccggtttgatgaactcaagatgacaaatcctgccatacaaaatgatttcagctactatagaagarcattgagtcgtatgagaattaataacgtcccggcagaaggagaaaatgaagtaaataatgddttggcaaatcggatgtctttgttttatgctgaggccaccccaatgctgaaaaccttaagtgacgcaacaacaaaatttgtatcagagaataaaaatttgccaatagaaaataccacagactgcttaagcaccatggcaagtgtatgcagagtcatgctggagacgccggaatatagaagcagatttaccmatg

SEQ ID NO: 182 - DNA, 192 bp, rat
  misc_feature (1)..(192): Clone #25  (PLSSH)
acacgataattctgtaacgtgtggtaattctacaagagttggagctgtgaaacgcaagtyttccgagaataatggragtkcagtttctaaacaagcaaaatcttgctctgargggaggaactatwtgccatgactggtatagaagacctytcagggaaaactcttcaggktttctcccagtatgtgtcctgt

SEQ ID NO: 183 - DNA, 243 bp, rat
  misc_feature (1)..(243): Clone #27  (PLSSH) n is c, g, a or t
accgcacagggattgtcaatgcatggacagccagcagctatgatagcvagccctggntccaggtggactttctgcggaakatgcgggtatcaggtgtgatgacacagggtgccagcygtgccgggagggcggaatacctgavgaccttcaaggtgkgcttacagbcytckatggtacgcaggttcgwrtttcatccaggatgaaagcgtgaaccggagacwaggaggtttatgggtaatcagg

SEQ ID NO: 184 - DNA, 162 bp, rat
  misc_feature (1)..(162): Clone #29  (PLSSH) n is c, g, a or t
acacaagtcaagcagagttccaataaatgtatganagtgaaaacaaaggcctatctgcagtcttaaaaaatatccccaagggcagagctgttcacgtgagcagggggaataaaaarccttttggggtgtcccaggcagggcaggattctkggagactcaggt

SEQ ID NO: 185 - DNA, 157 bp, rat
  misc_feature (1)..(157): Clone #31  (PLSSH)
acacagtttacagctacaaaaacctactttggtatttattacagaaaagtgttcagttaattgtgttattccttcagcaaatattcactgacacaaaatcctctgcagcattttacagcatatgcagcctcgtgcaagtttagatgagtggatttgt

SEQ ID NO: 186 - DNA, 288 bp, rat
  misc_feature (1)..(288): Clone #33  (PLSSH)
acaaacgaaactgactttgtggtgaacttcatgaccagcaatagaaactctcacacctccttcatctttaatcaataaggactgcaaatttgggcccacagccaattagttcattttgttagtttatattctcctcccattccaactttcttttttcttaccaataaatgaatgaaggcatttttgttggaaatgccgaaattggaagaaaaaatttgaacaatgtaaatttgatcaacagaagcaaattgaatagagagaaaggaacttaacaaaatttcagtaccg

SEQ ID NO: 187 - DNA, 212 bp, rat
  misc_feature (1)..(212): Clone #34  (PLSSH)
acttttatctgcactgccatcttgctggbccacttttctgtccctccgagtaaacggtcttagtcgtctgggtccccatcacctgcaaggactgcgcttatttatgaacagaacgacgtggaacgcgtgtttatttattgaagtctgaagacttaacagctggtatttattgtaattcattctgactttctcagtctattctgacagagagt

SEQ ID NO: 188 - DNA, 183 bp, rat
  misc_feature (1)..(183): Clone #35  (PLSSH)
acaaaaattctcccagtgaatcttttaatttgtaagagttgaaccaaaacttatatacatctctatgaagattagttatatacttcaaaatagtctgtgtgttcttgctacgttgtgtgagtgtaagctgcctgtcacttgtgagacttgtaagactacaagggcagtatgktacctgcccgg

SEQ ID NO: 189 - DNA, 105 bp, rat
  misc_feature (1)..(105): Clone #36  (PLSSH)
acctcaaagtagttctgggagtgtccctccggtaggtaggtagagtgcctacctagcacgtggaaggctctgtgcctgattgcctgcactggggggaaagtaagt

SEQ ID NO: 190 - DNA, 157 bp, rat
  misc_feature (1)..(157): Clone #37  (PLSSH) n is c, g, a or t
acagcttcactggaggtatgtctgacttcaactacctacacaccaactgctttgagatcacagtggagctgggatgtgtgaagctcccaccagaggaggcgctctacggcctgtggcaacacaacaaggagcccctactgancttcctggagatggt

SEQ ID NO: 191 - DNA, 193 bp, rat
  misc_feature (1)..(193): Clone #39  (PLSSH)
ggtactgattttggatggtgccattctgtggagctcggcaattgtaagtgacttggagtttccaccctcttttctcttgcaggacttgggctccattcggtgcaactgttggggtctagagtccatctttcacattcctgtccacgaaatttctcagggcagccatttcagattcagacagtgagtacctgcc

SEQ ID NO: 192 - DNA, 202 bp, rat
  misc_feature (1)..(202): Clone #40  (PLSSH) n is c, g, a or t
actancgggcgacttgggcctctcatgatgcatgcatcagagcdgaygcacagtgbtgatgtgcathcdgctgcaggaattctcgtgcctttcgacgccgcgtgccgcchcggtgcaggcgttacttctgtgtgcctatrtdtacattatatatayatatatatgtaatatayatgtayctcatatgtcctcacagaatcta

SEQ ID NO: 193 - DNA, 225 bp, rat
  misc_feature (1)..(225): Clone #41  (PLSSH)
acgagctgtgcccaatcctgtgatcaacccctaccagccagcacctccttcaggttacttcatggcagctatcccacagactcagaaccgtgctgcatactatcctcctagccaaattgctcaactaagaccaagtcctcgctgactgctcagggtgccagacctcatccattccaggatatgcccggtgctatccgcccagctgctcctagaccaccatttagt

SEQ ID NO: 194 - DNA, 505 bp, rat
  misc_feature (1)..(505): Clone #42  (PLSSH)
acggtgcagtctagtcttgtgtctaagtttacaggtatcctcttggacatcccgcatcccacacctttccaattagcaacagagagcatcctgtcggagagacctgaccagtcatgaaggaccgcctgtggaagagaagagtctgcaggaatcatgctacatattcagctggagagggaacacgcatctaatgggaatggattgttttgcttgtttcagagttttgctgttgagttgtcaaacattgcattgtttttcttttttgtktttttcttttttcctatagtcaattaagaataggggatagataatcataagtattttgggctgggagggactgttaagtaatcataggtgggtgggkaatttaggaaattaagaagttaggataagttaggataagttaggataagttaggataagttaggataagataggatatgttaggataagttaggataagataggataaggtagatcacataggataagatagattacatag

SEQ ID NO: 195 - DNA, 180 bp, rat
  misc_feature (1)..(180): Clone #43  (PLSSH) n is c, g, a or t
actgcatatttgccaacttctccgtgacaggtgggaagtggatcgknnntggctctgaagataacctggtatatatctggnatctkbagaccaaggagatcgtaccccccacacaatacactctaggggaaggknnnsgctggtgtgcagcagcaggctggttaccbantacctgcccgg

SEQ ID NO: 196 - DNA, 182 bp, rat
  misc_feature (1)..(182): Clone #44  (PLSSH)
accatgttgcatgtggcttcctctggatatatctaagcccctctgcacatctacacttagatggagttggtcaaagggaacatctgggttatgccttttttacagtagctttaggaaccgtcggcatgttgctgttgaagtgtggagttgtgagcgtggactgtggacagtcgacagcgtgt

SEQ ID NO: 197 - DNA, 198 bp, rat
  misc_feature (1)..(198): Clone #46  (PLSSH)
accgaaaccaatagcatgtttctgagattctccctgttcttcccctcagkcccatggtttctgggaatgcttaatcccttyttcatgttctttccacygaggtgagaaccctgtggagagcagtcagttcatggctctaaagaagtctcagccaccagcccctgattgytagkttccttgcagttcatagggagttct

SEQ ID NO: 198 - DNA, 256 bp, rat
  misc_feature (1)..(256): Clone #48  (PLSSH) n is c, g, a or t
acttaatcggcttccttgctcgtccggattttgctcgaggagccacagtgtcatctaagncacatggnaatcccgctcctctcccttgggtttcttgcttgtggcaccttthganatcgccttctcaaactcagagtccgagctgtcagaagaagatggcttcctcttgcgactgttcttggctttggcaggagcgggctttttggagacatgagcattcaaggctgaatctgacttggtctcctttggtacctgc

SEQ ID NO: 199 - DNA, 448 bp, rat
  misc_feature (1)..(448): Clone #49  (PLSSH)
acaatctcaattctatcaatttggcctttaaaaatcaccaagagaattatcatattaaaagaagaacctaattggctattgattcttgttcactgggaatcagaaagcttgtcaatcactgagtgttttccagtcttactttgaaatggtgcatttgtgtttctgatttactttatgaaacattgcttcagtttacctcagatctccatcctatccttcatgcattttttaactcatttgaacttcaagctgcagagggcctagaaatgggtcatcatctgcagccccggcatatacacgcggacatttgccactcccgcaagcaaatgattggagaagttgaccacctcagtcaagaagatcatgctggtgtgagttcatagctcgcagaggacatccctgacactggattgttctgaagcttgccagtgactgtttggcagtgtgt

SEQ ID NO: 200 - DNA, 241 bp, rat
  misc_feature (1)..(241): Clone #50  (PLSSH)
acccatgccagttcatagcaactctactgttttgagaataagactgacaagcataatctctggtcttggtttcaatgggtgctgtccaatgagcaaggggcatatacatccctgaccccctttgctaggttgatgggagagaacttcgtgctgtataatgtttcagctaccagaaaaaggtgataccaggttgatcaatattcaatataataacctcttggtaaataatactgccactagt

SEQ ID NO: 201 - DNA, 222 bp, rat
  misc_feature (1)..(222): Clone #51  (PLSSH)
acactgaggcctcagtctgccctccagacagccactgaggatgaccgcgctggcgaacgtccatctgcgcaaaggagcaggtgtcccccacaccgacgacctccacagtaaagtttcggaagadgtctatgakytccagggcccgtgggcgtaggcagaagatgagaatgaggggtgtaacaatgggactcagcaactcctccaagatgaacactgccttgt

SEQ ID NO: 202 - DNA, 104 bp, rat
  misc_feature (1)..(104): Clone #52  (PLSSH)
actgacgaatagcaaagaagaggccctaaccatggccttccgtggtgagcgagcacaggggagaatatcctggaggatctgaccaaagccatcattgaggatgt

SEQ ID NO: 203 - DNA, 149 bp, rat
  misc_feature (1)..(149): Clone #54  (PLSSH) n is c, g, a or t
acagtttgttcaaggaatttttgttgaaaaatatgatcctacgatagaagattcttatagaaagcaagttgaagtagatgcacaacagtgtatgcttganatcctggatactgcaggahcggagcagtttacagccatgagagatytgt

SEQ ID NO: 204 - DNA, 255 bp, rat
  misc_feature (1)..(255): Clone #57  (PLSSH)
acagtaagacacacacccacacacgaactgtatgtttccgcataagtatgaacaatatgttttcaacggtataataaacagttaaaatttttataggagttaaaacaatgccccattatttaaaaactggctcagtaagtctaaattcaagatgcagctgtgtcaggcttctctaaaccagatgacggaaacgcagggaagacaggggcagggttctgactagcagttacaactggaggaagggatgaagacaat

SEQ ID NO: 205 - DNA, 160 bp, rat
  misc_feature (1)..(160): Clone #59  (PLSSH)
acaagcttttttttttttttchttttttttttttttctcctttgagagtataatgaacatggtcacaccacaagtaaagtctgaagtaggacagaaaacgctccgaaggctggtttggtcacccgttatcattaaaaatggcagacccctaacaatatgt

SEQ ID NO: 206 - DNA, 115 bp, rat
  misc_feature (1)..(115): Clone #60  (PLSSH)
acacttagtgtaattgcaacactccaacaggaatcaaatcgaaccagcagaaccgtttctgcatctatgacttccatgtagagcacacatgcacacacgtctatgacttccatgt

SEQ ID NO: 207 - DNA, 243 bp, rat
  misc_feature (1)..(243): Clone #61  (PLSSH) n is c, g, a or t
actcataaatacaagcagcttgacattggcattaacaatmwtaanattagacgagtgtcttacacatgcaggtctggcgtggaactcagaacttagcctgccaagataggrgagcttatgcttttcgttgaagtgtgtgtgtgtgtgtgtgtgtgtgtgtgtgtcataaacagcccacagtagaatacgatgactactctcattcttgtgattaaagctgcccttctacacactgagtcttgt

SEQ ID NO: 208 - DNA, 135 bp, rat
  misc_feature (1)..(135): Clone #62  (PLSSH)
acctcagctccgagagacttatccctcagagagtcatggaccagcgcaagctcagcagggaccagtgggaggaccggatccaggtgtggcacgcagaacaccgagggatgctcaaggacagtgctatgctcgagt

SEQ ID NO: 209 - DNA, 285 bp, rat
  misc_feature (1)..(285): Clone #63  (PLSSH) n is c, g, a or t
acttcatcagcaagcgcctcaaaagcaggcaccacctgcaggatgatgtccgggtagacctctatgaagcagccaacaagtgggtgacagccgtgggcaaagaccggccattcatggggggtcagawgcctaaccttgctgacctggcagtgtatggtgtgctgcgagtgatggagggtctggaagcctttgacgacctgatgcgacactcacacatccagccttggtacctgkcccgggnsggccgctcgaaagccgaattccagcacactggcggccgttact

SEQ ID NO: 210 - DNA, 265 bp, rat
  misc_feature (1)..(265): Clone #65  (PLSSH) n is c, g, a or t
acactatgagaactacagamgcagaaaactagcagcagtgacctacaatggagtagataacancaagaacaaagggcaacttaccaagagccctctggcgcagatggaggaggagagaagggagcacgtggccaagatgaagwwbatggagatggagatggagcaggtgtttgagatgaaggtcaaggaaaaagttcaaaagctgaaggactctgaagccgagctccagcggcgccatgagcaaatgaaaaagaatttagaagca

SEQ ID NO: 211 - DNA, 347 bp, rat
  misc_feature (1)..(347): Clone #66  (PLSSH)
actaatggcatggcacgtgtcacatgcagtccatgtgggattctacatgaactgagtgagttttctgacgtcgcccatccctttgatgctgaaggcagtgcatctgtggaaagaagggactgcagattggggtcactgctctctgcgtcttccagtttctctgtgttatcttcccagttaatgtggaccttgtgactctgggattagaagccaaaatattcctttgaagcttagaaaagtctggttttactggtgggttttcccttaattctgtttcagtatattcattatttacatagtagccaactctaataaattcttaacctcggtaggtgcaggtaatcagt

SEQ ID NO: 212 - DNA, 906 bp, rat
  misc_feature (1)..(906): Clone #67  (PLSSH)
acccaaggacttggggcttttttagagcactgggcatcgtcttgaagatagaacgtcgaagaatgaagtctcttgctactaccttgaaacatttgttcaacctctctttctgtttgattctcaactactaccattaaccctgctgcaagatttgtcaccgttctgcctgattgctgagactccgttttgctgctgtgcaagaagaaagaggcaggcctaagggagcatgcgtctcagcacagtctcccattgccaaacacagtttgagcagtaggacataacatggtttttatttcagtttacctgtcgaagaagaatcttgatggtgaagtttctcttccaccagcgttatctctttcttaacctagcatgttttaagtagtttagtggaattttttttctcattggtaatggtcttggtaatttgttttttttttaaaggrttttaaagtgttgactatgccttcagaaattctgttgratcacagtacccgcctccggaacaagccctaagccgaatctgcagatatcatcaaactgggggccgtcgagcatgcatctagaggggccaattcgccccaaagggagtcggatacaatcactggccgccgttttaaacgccggrctggggaaaccttgcgttacccacttaatcgccttgagaaaaccccctttgccaattggggaaaacgaaaggcccgmccartagccttcccaaaattgggcgctggatagggaatggggscccttaagggggttaaccsscggggttggggtaacccgggmccgtaaaatttaaggcttaacgccccgttwagtgttccacccttttccaaattcccggttccccgaaagaaaattgggccytaaggcgccttaaaggtttaggcktscccaaaaatwtta

SEQ ID NO: 213 - DNA, 84 bp, rat
  misc_feature (1)..(84): Clone #68  (PLSSH)
cctcgtgcatgwccwcwwcaaggcccagcatctccagcagtggggagaacgagtctgctcagagcaccgcaagcacggtgcagt

SEQ ID NO: 214 - DNA, 96 bp, rat
  misc_feature (1)..(96): Clone #70  (PLSSH)
aacgggtcctagctaagtcagagaaagcagggaaacaaactggcatctgcacaccgtcttcaagtaaacagagtccctgacgcacccggagagtgt

SEQ ID NO: 215 - DNA, 164 bp, rat
  misc_feature (1)..(164): Clone #71  (PLSSH)
acaactcttatgctgaaaagcccttggagggaagtgatcatgcagggaggttgtcagggtgtagaaaaaaactccacaaactgggaagattaaccccgaatgcttgaatggataggtgtgccctcaaaccagctgcattatcacccccacccagccagaaatgt

SEQ ID NO: 216 - DNA, 127 bp, rat
  misc_feature (1)..(127): Clone #72  (PLSSH)
actttagttttataggccctcacaagtgttaattctcctgtaagtttctctacatgagccactgtagtaatgtgcttagatcttagacatagtctttatcataacaatattaactagcaccagaagt

SEQ ID NO: 217 - DNA, 317 bp, rat
  misc_feature (1)..(317): Clone #75  (PLSSH)
acggtgatggaggacctgcctctgaagcagagcagcgatccatccaatgctgcctggatgagcctctgcctgcttgctttgaacaactcctgagagtttatacctatttatttcccccaaattctatgttaatattttacatgtagaaaaaaaaaccctaagtgttdattgaatctacttgcaagattgcatcttatttaaagtttatttaatgtcaggtctaagtttaaccaatccttattcttaatttgaaggttaaaggtttccagatcccctcagggaattgtattaatatttcagaggcaattttscccggc

SEQ ID NO: 218 - DNA, 90 bp, rat
  misc_feature (1)..(90): Clone #76  (PLSSH)
acctggtgccacaggcccgcaccctgtdtggcctagacgagagcaaggcccaactgtcagcgacagtgctcactctactcatcaaacagt

SEQ ID NO: 219 - DNA, 229 bp, rat
  misc_feature (1)..(229): Clone #77  (PLSSH) n is c, g, a or t
actcaagaccaaagcacataatgctaatgtagggctcagaggggaaatacagttctcctgcataattgaggaaatgtggagtcctttcaagaaaatgtaataaacataatcatcgtagcctgctgacaccaaggaaaaaggcccctattcgctctttcttttatgcwgtaatttgctggtccctgctgatcttcaaattggatcactgtagtaaattatccntgctggt

SEQ ID NO: 220 - DNA, 83 bp, rat
  misc_feature (1)..(83): Clone #78  (PLSSH)
gacagcagccaggaatggattcttagcatcgaaggggggtttctggttctcgtagctcttcagacggcccatctcacccgtgt

SEQ ID NO: 221 - DNA, 217 bp, rat
  misc_feature (1)..(217): Clone #79  (PLSSH)
ttcgctggcgtcagtgtctgagtctgatctgattctgtattatcaagtaagctgtatggattgcggtctggatctttgagcacagaactgatggaggatttgccaccacgtgtccaccacgacctcgtccccagaaacacttctgcctcttcctcctgggcgtctcctgctgtctcgctgatgtcgagtctctcgatcatcttctcctgcatgacat

SEQ ID NO: 222 - DNA, 172 bp, rat
  misc_feature (1)..(172): Clone #80  (PLSSH)
gaaacagtatcattctgcttgagggtaatggtgggtgtmataatactctatgaccacgtccatccagtgggagcatttgccaaaacttctaaaattgatatgaaaggttgtatcaaagttcttaaggatcaacctcctaatagtgtagaaggtcttctcaatgctctcaggt

SEQ ID NO: 223 - DNA, 94 bp, rat
  misc_feature (1)..(94): Clone #81  (PLSSH) n is c, g, a or t
ttgaaatggctcggaaacttgactcgccgttatcaaggaggcaagadagcggctactataactganattgagaggtgcctcctgcaccgggagt

SEQ ID NO: 224 - DNA, 243 bp, rat
  misc_feature (1)..(243): Clone #82  (PLSSH)
acaacacagttaatttaaacattttcattttggctgcacatgaaaaagcggcagtagaaaataaatttatgagggatttaaatagcagaataggcagttttgccatgcaggagaagcaatattaaatattagttttcaaaaaaaatccacatttgaaaatatttagttcaagtcacagaacttttctcagtagagacctcaatgcaatgcgtaattagctgccttagatggcctgttttgaaa

SEQ ID NO: 225 - DNA, 126 bp, rat
  misc_feature (1)..(126): Clone #83  (PLSSH)
actgctgcagtcgaagcgcggtcycatctcgcttctccagaacccgcacaaagctccagagcggatcctccacctccggctccacgacggacggtattcccgctcctcggcggacgatattccagc

SEQ ID NO: 226 - DNA, 269 bp, rat
  misc_feature (1)..(269): Clone #85  (PLSSH) n is c, g, a or t
actgtccagacgccttgtatgaagtgatgctaaaatgctggcaccccaaagcagaaatgcgcccatcgttttctgaactggtctccgggatatcctcaatcttctccactttcattggcgagcactatgtccatgtgaacgctacttatatgantgtaaaatgtgttgctccatatccttctctgttgccatcccaagacaacattgacggcgaacgaacacatgacggataagaggcccgccagcccccttctaagaaacagtacctg

SEQ ID NO: 227 - DNA, 148 bp, rat
  misc_feature (1)..(148): Clone #87  (PLSSH)
actgtggttcttgctgggcccacggacgcaccagtgccttggacgatcgcatcaacatcaagagaaaaggtgcatggccctccaccctgttgtcggtgcagaagtgcatcgactgcggcaatgctggttcttgtgagggggcaatgac

SEQ ID NO: 228 - DNA, 179 bp, rat
  misc_feature (1)..(179): Clone #88  (PLSSH)
acaggatttatactataacatacttgattgggattttgaaaatagtttaaacaggaagatggaaaacaaaactttcatctagagactaaattggaacagaaacagtctgttaaggagccggccgttccgcctgcttcacccagcttagggaggttaagtctacattcccaccactgagt

SEQ ID NO: 229 - DNA, 288 bp, rat
  misc_feature (1)..(288): Clone #90  (PLSSH)
tccagaagcctcgacattagatttggtgacacgatccctctctctctttgggtcccctgtccccgactcccattaacaatagctacttcccacacctgggacaccttgaagtccgacgacttctcacactgggaatagctcccttgttcttgtggaatccacctgccaaccacccatccacggactcatccatccttccatccgtcatccatccatccgtccgtccgtccgtcctgaytttcctagtgccactgtctggctaggcaytacccactatcaacctggttc

SEQ ID NO: 230 - DNA, 221 bp, rat
  misc_feature (1)..(221): Clone #91  (PLSSH) n is c, g, a or t
acctttggtggcttgggatcaaagagcaaaggtcattatgaggttactgggagtgatgacgaggcaggcaagtbacaggggagtggagtgtccttggcctcanagangtcccgactgtcttcctcctccagcaatgacagcgggactaaacttggcatccagcttcctgaggtagaaatatcagtttccacaaagaaagagtagtgggccttcatgcatgt

SEQ ID NO: 231 - DNA, 89 bp, rat
  misc_feature (1)..(89): Clone #94  (PLSSH) n is c, g, a or t
actattattthnmattgtaaagaggcaggaaagtctcccggctttgcggatgacaaatgaatatnagccgcttagtttgtgccgtaggt

SEQ ID NO: 232 - DNA, 320 bp, rat
  misc_feature (1)..(320): Clone #94  (PLSSH)
accggaagaagcagctggcgaacagctccctgcacatgaccaggacccttctaagtgccacgagctgtctcccaaadaggtgaaggggatggaacagttcgtgaagaaatataagagtgaakctctgggcgtgggcgacgtgaagcttccttccgagatgaatgctcaaggtgacaaggtgcacaaccctgccggagacagaaacaccccagcagcagtgggttccaaggacaagtctgcagaagccaagaagacccaatattcctgttactgctgcaaaaacaccatgagggaaggagccccagccatctatgctgaaa

SEQ ID NO: 233 - DNA, 252 bp, rat
  misc_feature (1)..(252): Clone #95  (PLSSH)
acttatacgtcgtcatccaagccgcaaagcccaagtattactttgcctggacattggcggacgcagtgcacaacgcagccggcttcgggttcagtggcatagatgcggatgggaattcttgttgggatctactatctaacctgaacatctggaagatcgagactgccacgagtttcaaaatgtacttggaaaactggaatattcagacatctacatggctgaaatgtgtgtgctatgagcgggttccctggt

SEQ ID NO: 234 - DNA, 296 bp, rat
  misc_feature (1)..(296): Clone #96  (PLSSH)
actgtttttttcaaatcttagcagtgaatttcagtattatcatctcacaaggtttcaaagtccacatcttaccagaagatttgtggatttcttcaatttactttaatttagggacccttcttcaattagctttaaaaacctgtaagattggtcataagaaaatataaaatgaattgacatgtcttcactggagggaagtagacaaaatgttgcaggtctcattgtcaccactgtcatttaatctgctgtgatggaagcatgacctgtatcttaatgtagccacattgacactggac

SEQ ID NO: 235 - DNA, 134 bp, rat
  misc_feature (1)..(134): Clone #98  (PLSSH)
accatgagagtctggcttgcgagcctgttcctctgcgccttggtggcgcactctgaaggtggcagtgaacttggagcttctgatgaatcaaactgtggctgtcagaacggaggagtatgtgtggtcctacaagt

SEQ ID NO: 236 - DNA, 856 bp, rat
  misc_feature (1)..(856): Clone #99  (PLSSH) n is c, g, a or t
yccctccytataccacaacaaaatacaatctagcaacaacmngcagygtgctggaattccggcttacgagcgkggtgcgcgaggrcggtacttcckcaaatcattagcwgcacragtgtgctgcgtatgaaaacgagttactggagatakccgatcacacgtgkcrrccgcwcgagcakgcawctrgackgcgcaattcgccctatagtgagtcgtatgacaattcactggctgtcggtttactaacgcagtgattkggaaagrccaattcgttmccaacttgaatcgcttgcagtcattgscgttggtaacagggtaatactgggaarcccgcaccgttagcccttccatcagttgcggccagcacsgtkaatcacgtgcctagtagcgcsgattaaggaggggggnctgtgcgtacagtgggacctggtatcactatcagcgcccatggtacgggccgtttartttttcgcttcttgttttacacgttggacggtttacmcntgaaagtcgaawaacgggggtcctttagggttctgacttagttgtttacggaagttcgacsctaaaacgtcaatacgggawacgtcsccctgtkacgtnccctttaaagcttgtttgccccttcccataarrtcactacgktaaagtnggtttggttcaacccggaaaacgdattttacccggcatngtttcatttaaaagatttgcggtttgscatgggaaaaacaacggtttcggaaattwtccgatttaagraattcggtagggtttataaaggaccgttagacaatttaaaagttttaaccktggttacggaccggattcggaaggaaaaaccccgaaaaggggttcc

SEQ ID NO: 237 - DNA, 272 bp, rat
  misc_feature (1)..(272): Clone #100  (PLSSH)
acgtgtttctagcgggcggtgttttcgtctccttgttgctagattcctctcagatatcccggaaccwtccagggcccaagcagtatgacgtctgcctttggagaactacatcaacctgaactgttgcagtaataacttctgatggcwgaatgatcgtgggaaccctgaaaggttttgrccagaccattaacttgattctggatgarakcyacgwgcgggtgttcagctcttctcagggagtagaacaagtggtatwtggggtgtatgccatt

SEQ ID NO: 238 - DNA, 871 bp, rat
  misc_feature (1)..(871): Clone #101  (PLSSH) n is c, g, a or t
ccagcgctyctarcttagcgaatcggtgactwcttganagaggtccacngtayttytgagynytcaagctrccygcrgkcaagaaactgctkkattttatgccatttgawggycgaakgcctgwttycagggggcattccatgcckgaccamtgggcttaacgcccacgtcggcactgccctccagrccwcagcgctaagscgggattctkcagatatccttttacactgggggccagctccarcatacatctagagggcccaattcgccctctagtcgagtcgcttacaattcactaggccgtcgttttmccacttcgtgactgggaaaaccctggcgnyaccctacttaaacgcctttcagtaaatccccctttcagcaacttggggaataaccgaaaagcccgggacagatagccctcccgaaaagttgcgcctttgaagtgcgaatkgactccccctgtattgccgrttaaaccggccgcgttttcctgttaagccccgkaccgctacaaattacaagccccttacggacgctcccttggctttttacctttctttttcgcaagttcgccggtttgcccgtcaacttttaaatcggggccccctttagggtcccytttaggscttaaggccctccgtcccaaaagtctttttgggacggtcctcttgcccatcsccggatcaacggttttcccctttaagtccaagcccggtttaaaatggncccttttcaactgggaaacccaaccacctngggtttcttngcttaagggttttcctttgggcttggcnaaataacccctaaaaattytcccsgtttcaaattgggccaggtcctagaaggaggccaacccccccaaaggtggacc

SEQ ID NO: 239 - DNA, 96 bp, rat
  misc_feature (1)..(96): Clone #102  (PLSSH)
actgtggccttcccaggatgaaggacatcaatgaccatctgtdtcctctgargcagacggtttgtvatggacttcctggtccggatggttaccgtg

SEQ ID NO: 240 - DNA, 109 bp, rat
  misc_feature (1)..(109): Clone #103  (PLSSH) n is c, g, a or t
tctttagagmgcttcattgtcgacgtgtgcttctagcccagtggtcagtcatggbatgccccctgggtcaggccatcggccttcnvgnggctatcttccacaagthcct

SEQ ID NO: 241 - DNA, 282 bp, rat
  misc_feature (1)..(282): Clone #104  (PLSSH) n is c, g, a or t
acccagggaattataactcatgctgatgtcaccttaatgccatgccttgactcagttgtttgcatgatttccaatgttaccagatggatactctcttgtgctacctctgagcttgctatagacacaaaggctgtcttctttgcctgtgcacgcgcatccctgggtattttttaatgttttccgtgtatgctttcatacataactatgcactataaatattaaatggatgacttatatgatattgaaataaagcctctttcactttaacaaaaaaaaaaancc

SEQ ID NO: 242 - DNA, 165 bp, rat
  misc_feature (1)..(165): Clone #110  (PLSSH) n is c, g, a or t
accttgttgatctcatcctcatacttattcttgangtcctccaccaggccctgchtgttgccaagctccacctccagcttcagcttctcctggcccagggcttccagctgccgacggaggttgttgatgthacccttcaaacatgtttgtccakgttgctcctyg

SEQ ID NO: 243 - DNA, 210 bp, rat
  misc_feature (1)..(210): Clone #111  (PLSSH) n is c, g, a or t
acatattgttaggggtcagccatttttaatgataacgggtgaccaaaccagccttcagagcgttttctgtcctacttcagactttacttgtggtgtgaccatgctcattatactctcaaaggaganaaaaaaccttgtaaaaaaaacaaaaacaacaacaaaaaaaaacaatcttattctgagcattccagtaactttttttgtgtatgt

SEQ ID NO: 244 - DNA, 170 bp, rat
  misc_feature (1)..(170): Clone #112  (PLSSH) n is c, g, a or t
acgcaccaagaaggagcatcagaaagcctgggctcggaacctgaacggttttgatgtggaggaagccaagatcctcaggctcagtggaaaacctcagaatgcsccagaaggctaccagaacagattganagtttttgtacaagcwwttbnnnngagctcggtaycaaggt

SEQ ID NO: 245 - DNA, 504 bp, rat
  misc_feature (1)..(504): Clone #113  (PLSSH) n is c, g, a or t
acccgaattcccccaanknccatcaccctgggcggccgctacagnatgcatttasagggcccaattcgcccnataggggatcggannaaantcaagggcgacgtwtacaacgcgtgacngggaaancccggngttagccacntaatcgncttncaaacancccccctttnccagntgggcaaaantanaggnccgnaccggagcgccncccaaaagttaaaccnggatnccnnaaggggcccccgaancggccannaanccngnggcnnaganggtancgggggacggcnaaaagtgccagnccagcncnggcntttsncctnncccgtccccaanntnanggtnccganccaaaanngggnnccncagntccganaagnntnagccggnccaagaaannnagnnnnaaagggnnagggccaaaacgttnnccagaagggcagggntaaaaaggncnggcaacggaagncaccnaccnnagatngagaangcgggngcaanaaa

SEQ ID NO: 246 - DNA, 234 bp, rat
  misc_feature (1)..(234): Clone #114  (PLSSH)
acaggccgagcatttcccggtccagctgccacagcagctggctctcaatgggcaggtccacggtgctggtcttcacttttgccttctttgcttgaggtggttggtccatctttttatcttttgatccagcttgagaggtctccatctcctcagactccgccttgttttctgctggwgtctgtggctgttgctcttcagtatgtggttcttcctggtccacctgcatcttcttga

SEQ ID NO: 247 - DNA, 149 bp, rat
  misc_feature (1)..(149): Clone #115  (PLSSH)
accccgatacaggtgatgatgatgggcaacgtcaaagtggcagctctcctgctctcctatggcgcagattcgaactgcgaggaccccaccaccctctcccgaccggtgcacgacgcagcgcgggagggcttcctagacactctggtagt

SEQ ID NO: 248 - DNA, 78 bp, rat
  misc_feature (1)..(78): Clone #116  (PLSSH)
acgttgaccatctttgcagcagaagtgtctgcvatgaaaacgagagaccggcagcagcgatccccgcctcaaccgcac

SEQ ID NO: 249 - DNA, 389 bp, rat
  misc_feature (1)..(389): Clone #117  (PLSSH)
acgagatcaccgaccagtctgggggcgctggctgcctgcgcacccacctcaagatcacagabtctgctggccacattctgtatgccaaagaggatgcaactaaagggaagtttgcctttaccacagaagactatgacatgtttgaagtatgctttgagagcaaggbccasggcggatacctgaccaactcgtgattctagacatgaagcatggagtagaggcgaaawattatgaagagatygcaaaabttgagaaactcaaaccactgnaggtggagctacggcggctcgasgacctttcagagtctattgttaacgactttgcctacatgaagaagcgggaagakgagatgagggacaccaacgagtccacaaacacccgcgtcctgt

SEQ ID NO: 250 - DNA, 217 bp, rat
  misc_feature (1)..(217): Clone #119  (PLSSH)
acaaatttatatgactaagagtaattctaaactcttgaaaaactacagttatygatattttggtbaatatctaatatttatagacaattttctcaatgatttcttttatcattgacatcatttataacctgctataatttaatagtcgtaaacacytatgctaatcacttggcactgcatgtagtttbsssscataaaagtctacttttatacacgt

SEQ ID NO: 251 - DNA, 187 bp, rat
  misc_feature (1)..(187): Clone #120  (PLSSH)
tttacagacgtctcctgcacactaagctctcagtgtactacggagttcagaggttctgctcggatgttccccaagtcgagtgcagaatctgtctcttcatctatttccccaatgactgctacattatctcctctcacaatgtataacccaataccacttgttctactcctgagaagagctgaagacc

SEQ ID NO: 252 - DNA, 270 bp, rat
  misc_feature (1)..(270): Clone #121  (PLSSH)
gatctctttccttttgcggccatcgctggattgcagccgccaaaatgaagttcaatcccttcgtgacttctgaccgaagcaagaaccgcaaacggcatttcaatgcaccatctcacattcgaaggaagattatgtcttctccgctttccaaagaactgagacagawktataatgttcggtctatgcccattcgaaaggacgacgaagttcaggttgttcgaggacactacaaaggccagcagattggcaaagcggtccaagtgtacctgg

SEQ ID NO: 253 - DNA, 81 bp, rat
  misc_feature (1)..(81): Clone #122  (PLSSH)
acctgttgtcatcacctggtgtccaaatatgcaccgccagcctccagtgccccagtatcctcctccacagccaccaccgcc

SEQ ID NO: 254 - DNA, 167 bp, rat
  misc_feature (1)..(167): Clone #125  (PLSSH)
cagagaaatagcaagtagattttgtatgctttgttttccggagagcttctggcaaaatgcaagtagatgacttagaccctgtttgaaaagaaatcatgagaaatcagaaggcacttaggtcgtattctaggtataacaagtattccggtcacacagaataacaagtt

SEQ ID NO: 255 - DNA, 106 bp, rat
  misc_feature (1)..(106): Clone #126  (PLSSH)
gcaaccgtcagcctcagtcgcagccgggcctcgctcctcaacttggcaaaaatgcctacagagactgagagatgcatcgagtccctgattgcwgttttccagaagt

SEQ ID NO: 256 - DNA, 222 bp, rat
  misc_feature (1)..(222): Clone #127  (PLSSH)
cggttgggggatccatgaagtatgagtgaggaactggtagtgaattcagtattggccaagtccatgttgtgtgctcagaagtctctttaccaaagaccaggagcaggagctaagcatgtccagtcctcctcttgtgcgacctttgacagtggtctggtccacggggctgctcagawaagcgctgctcttgtatgtctcactggtattggaaawtttwcctgt

SEQ ID NO: 257 - DNA, 218 bp, rat
  misc_feature (1)..(218): Clone #128  (PLSSH)
cggtacttgagcatgactccccaggactcctgcctgcttcaagactcgctgtcacccccgagtcagcaatgcccacacctcagctccagagcagaagcagcgctcacaccgagcacctcagabcctgcctctcaggcctgctgagatggtaagcatgaggacacaggaacttgaaagggccagccagtcctgacaaaaccaaagttgtacctgcccgg

SEQ ID NO: 258 - DNA, 245 bp, rat
  misc_feature (1)..(245): Clone #129  (PLSSH)
acatagacaaaggtaaagagaaacaaccgaaaaagacagagctattgcctaagagaaagaaaaattatgtaatgtgtatatgcattccacagaaatatatacagggaaaacttccagaaacctcagtaaaccaagccacacttagaaaaaggaaagctgaattttattttatcaacagccattcttaaacatgaacatgcatacgtaataatctacgcacacatcacagtttttaacgttagtta

SEQ ID NO: 259 - DNA, 109 bp, rat
  misc_feature (1)..(109): Clone #131  (PLSSH)
accataagcaktttccatggggtcagagaatggctggakagttgtggaaaccakaggwggtattgttttggagatcaggacctcwctgtgtagccctggcctcaaactc

SEQ ID NO: 260 - DNA, 164 bp, rat
  misc_feature (1)..(164): Clone #132  (PLSSH)
gggtgcttccagttggaggggccctcagctatgaatgttccagatgaatgtcttgttctgggagcatggctctttccggattcaggcacccccgttcctgggtagaggcgttcagttcgacgtcgttgtgaacaagcctccagcctttctagttctgtgctacg

SEQ ID NO: 261 - DNA, 108 bp, rat
  misc_feature (1)..(108): Clone #133  (PLSSH)
actgcagcatcatgttggamagtmgcttggmggctgcagacacggagatgcgttccccattccgcagatagtmtagcctgcvttccttggccaacagcctctcccagt

SEQ ID NO: 262 - DNA, 745 bp, rat
  misc_feature (1)..(745): Clone #135  (PLSSH)
acatgtgatttgcactagaatgtgaaggcacactcacaagtcagtctgagagagcacttccttttcaagctgaggtgtttcgtgttgcagagaaccagtcgagttgctgctccttgaaaattaacggaatcatgaagatggagtttgaagatggtgaagtttcatagggtgcagcaagaggatcacggaagcctttctatactgcacctaaaagtctggcttccttcataaggtgcccgggatacggccaggtcacgtcttccttccttctcttcttcctcttctctccctctgtatgaaaccagcatgtcctgtgctcgtatcagcttctttgtaagacaatcggcagcctctgtttgagatggtgagaggttttaagacttaaaacgggggctatcaacttacaattggcagtcacgatgggaactgacttagatccgcatgcggttatgcctcagaarggctgaagatccatagcatacagtcgttgatgagaaccgcccagggtcagttgaggcaaaaagtacggctcggracaagccctaagcgawtctgcagaatcatcaaactggggcgtccggatgatctaagggccattcgcccaaagggggcgataaaataactggcgcgtttaaacgcggactggaaaccgggtwccaataaacgctgagaaacccnttgcaggggaaaagraaggccgacraggcttccaaagtggccgaaaggaatgacgccctagggctaa

SEQ ID NO: 263 - DNA, 153 bp, rat
  misc_feature (1)..(153): Clone #136  (PLSSH)
acacagataactacaggctggctggccggggggctaacctaacgttccacccggagcacagaacccacctctgctaatgttgcatgttcagtaacgccactgagaatactgcattgttagaagtcagttctgcaccctttgacagctgctgga

SEQ ID NO: 264 - DNA, 288 bp, rat
  misc_feature (1)..(288): Clone #137  (PLSSH)
acgaaaaaagacataaaaatattccctgcagtgaaaaaaaatacatgtaaacatctcctccaagtcactggagaacccttgasssygtccattatgtcttcccaatgtcccacaattctcttggtcatcttttctctgtgggtatcagtctgccttcttctttacagaatttggactctgagactgcctttgaggctggcgtggtttgactggcaggagaaggatgatcagcagacataggatgtgcaagaaaaagaacatggacttgtataaactgatggttggctc

SEQ ID NO: 265 - DNA, 100 bp, rat
  misc_feature (1)..(100): Clone #139  (PLSSH)
accattggccctgcttcccgatctgtggagatgctgaaggagatgattaagtctgggatgaatgtggctcggctgaattcctctcatggdacccatgagt

SEQ ID NO: 266 - DNA, 136 bp, rat
  misc_feature (1)..(136): Clone #140  (PLSSH)
gcggtagctttccatacaattagatcagacaggaagtcaataagacagacgaaacagggcctgttgcatttcatagccgctatttagggctgcagtggtttcatgtggatttacccactcagtatccacaagctgt

SEQ ID NO: 267 - DNA, 183 bp, rat
  misc_feature (1)..(183): Clone #141  (PLSSH)
accgccagtcagggttcttgatgtgctctttaataaagggaaggacatgtggtacaatgtcatcttcacagcaggtagacaggagcatgaggcacaccccagctgctttgcaagggttccagtcatcgtcgtcatcgttttcatcctgtttagttagtgtctgtgttwggatgggcaccaggt

SEQ ID NO: 268 - DNA, 268 bp, rat
  misc_feature (1)..(268): Clone #145  (PLSSH)
acataagtaactggtgtgtgtgcacaagcatattgctctttttatcttttccttttttaaactaaatggccgatgttctgttttggttacatcagatggagacagtctggggacagttctggttctgtgacaatatcccccttctccactagtggcatgctcagtcagctcswatctctctatccagtaagttattttgctctcattgttttaacaaaaggantcbaacaacacaaaatcctcgcataccttgttcgattggagaatt

SEQ ID NO: 269 - DNA, 175 bp, rat
  misc_feature (1)..(175): Clone #147  (PLSSH)
ctgaaatggtttgtatggcctttcagcttgttttaagataaccgcttacttgtgctttaaatggcattttgcacaacgcatgcttaatatgatggaaagtttctgcttttcgtttctaaaatgaacactttaagtcgtcagagcatcagttaaagacatatgtttgacattttaa

SEQ ID NO: 270 - DNA, 107 bp, rat
  misc_feature (1)..(107): Clone #151  (PLSSH)
gctgtggagaagggagcagtatgaggggtgttctgtaggaacagaaccggcggcttatgacttgaggcttcctgactgtgttttcagatttcaggcaacttggctgt

SEQ ID NO: 271 - DNA, 348 bp, rat
  misc_feature (1)..(348): Clone #152  (PLSSH)
awgcmtgacatctcttgcrgmcgaccacatcwcactgacagtgccgcwcnattcaggcwtmkaggmgtggtcmagktcggcctgatacattaatcagcattaccattacmaytggccnnttnnnnnnnnsgtgtccctcttgagaacaaggtctggtagatctgcacttggtcagcagcctgggctgtcytgtcagaggtgataaaaaatraacccctcttcaagatggtctacttggctgtaaatattyngagctttggcatgcctggtctctttcttttttctttttctttctttttttttccctttttctactaatgatgcccaaataaccaaaaaacacactga

SEQ ID NO: 272 - DNA, 193 bp, rat
  misc_feature (1)..(193): Clone #154  (PLSSH)
acacgttttgtgtttcttagaggtttaaaatggcatgctttgcatgcaaaggaaagccattgcaattagtctttkattatacctgtatcttgggttttataaactgctggcttgagttttgttgctcttaaaccacagtacctgkcccgggngggccgctcgaaagccghattccagcacactggcggccgtt

SEQ ID NO: 273 - DNA, 134 bp, rat
  misc_feature (1)..(134): Clone #163  (PLSSH)
actgtggaggtgcttcaaaggcaggatacacggcaatctctggcacatttctattgttgatgtatttktdgcagttccaaacacaattgattaggtaagccttaadddtgatgaataccacaaagaacacaagt

SEQ ID NO: 274 - DNA, 250 bp, rat
  misc_feature (1)..(250): Clone #169  (PLSSH)
acttcccacacaccgggaatatgttgccagttatgaaaaacctaatgcacttttgtttcccttttttcttcttcattttaaaagaaggatattattttttgattgcatttaaaatctagacagggggacatgcaaaaacaatcatcatccacttggatgtcattttataaattaacactgtgtgcttttgtatggaaacatatataatttaatagtataaganaagatatatattcactggcattcatgc

SEQ ID NO: 275 - DNA, 315 bp, rat
  misc_feature (1)..(315): Clone #170  (PLSSH)
acaagcwttttttttttttcttttttttttttttaagtcattgtatctttaataaccgggbwgtaaatcaaaacatggaaaaaagtcgttccttttacgggttgatgcccatcaccacagactttagtgagtgagaggggattctaaacagcatgggaaggcttccaggctcacgggatgaagtgtttggttgctgataatcggccgggggacgagtaacacaagagattaacaagccctcagaggwcaggtcgcggttkctgggtcatgagcaccttgaagcgtttcttgatggtgattcggtgtcttgagtat

SEQ ID NO: 276 - DNA, 138 bp, rat
  misc_feature (1)..(138): Clone #171  (PLSSH)
gtacacttctgggtggccgaagaatcagaataggtgttgataaagaattgggtctccacctccagcggggtcgmagaaagtagtatttmnatttcggtctgtaaggngtatagtgatacctgccgtaatcatggtcat

SEQ ID NO: 277 - DNA, 173 bp, rat
  misc_feature (1)..(173): Clone #176  (PLSSH)
gggcctctagatgcatgctcgagcggccgccagtgytgcatggcatatctgccwgcacctcgkgctttcgcagcggtccgcccgcgagcagtgtacggtaacacaggtttacttgtcacacstggtctttagttcctcagaatttcaattttggagattaktgccgtgtaava

SEQ ID NO: 278 - DNA, 120 bp, rat
  misc_feature (1)..(120): Clone #179  (PLSSH)
ggcggttaacatgcagatgctccctaagaggaagcagccagtgcgncgnttcagagcgagcgagtgcgacagtacctgccctggtgcggccgctcnncagaccgaattccgagcacactg

SEQ ID NO: 279 - DNA, 190 bp, rat
  misc_feature (1)..(190): Clone #181  (PLSSH)
acatttgccagggcagatcgatacttcagaatcagctcatgcagcagatgcctggcacctgctcagtccatctagaagcatttgcggtggacaatggaggggcctgactcgtcatactcctgcttgctgatccacatctgctggaaggtggacagtgaggccgggatggagccgccaatccagactgagt

SEQ ID NO: 280 - DNA, 174 bp, rat
  misc_feature (1)..(174): Clone #185  (PLSSH)
acaagchtttttytttttyyttyycttttttttttttttgacgtaaaatagaaacagactttatttctctggaagaagcagatatccatggctgggacagcagctttggcaacagaggcgatgggaacacatcaaatggacacaggggaggaacaggcatcaaacwggacaagt

SEQ ID NO: 281 - DNA, 153 bp, rat
  misc_feature (1)..(153): Clone #192  (PLSSH)
tacatgtctggatatcttaggtccaacagactgaactagtcttgttgacaagggttcccaagcaatggatggtttgtctgtctgattcttccacatttacaggaatcacatttgggcgcgtgtgcaagctatgctgtagctagttcaggtacc

SEQ ID NO: 282 - DNA, 160 bp, rat
  misc_feature (1)..(160): Clone #196  (PLSSH)
attgtgsycctctakatgcatgtctcgwagcggccgtcctavtgctgtatgcgtatgatcctgtcgaggacatttcgtgcctttcaggtgctgtgttgsctgtcagrgaagtgtgycgrgtatctttatactgtatgtgttttctaggttataacatatt

SEQ ID NO: 283 - DNA, 177 bp, rat
  misc_feature (1)..(177): Clone #200  (PLSSH)
cgggcgaattgggccctchagatgcatgctcgagckngbcgccaatgctgmayggcmtcatcygccgcagmttcggtctttcgcagcggsccgtcccgggccagggtactccccacacaaatacactctakgggarakggmghkatactggtgytgccagcagyctagctggcctac

SEQ ID NO: 284 - DNA, 197 bp, rat
  misc_feature (1)..(197): Clone #201  (PLSSH)
acttgggaagggcgaggtgtctttgtccattcgacttggacagttcctaggccaggtaatcaggtggcaaccggagaaaaggggacgccaggctgtctgtsctcttccagcctagcaagcagactgcgtggcgtctgtgvccatctggnaggtggggtggtctctgaggcgaggctgcacgctggacgggtggdkta

SEQ ID NO: 285 - DNA, 183 bp, rat
  misc_feature (1)..(183): Clone #206  (PLSSH)
ctgagcaggattaccatggcaacaacacatcatcagtagggtggggctaacctgtctcacgacggtctaaaacccagctcacgttccctattagtgggtgaacatctaacgcttggtgattctgctcacaatgataggaagagcgacatcgaaggatcaaagcgacgtccgtatgagcctgcg

SEQ ID NO: 286 - DNA, 205 bp, rat
  misc_feature (1)..(205): Clone #207  (PLSSH)
agctcggtaccaagcttggcgtaatcatggtcatagctgtttcctgtgtganattgtntccgctcabaattccacacaacatacgagccggaagcataaagtgtaaagcctggggtgcctaatgagtgagctaactcacattaattgcbttgcgctcactgcccgctttccagtcgggaaacctgtcgtgccagctgcattantg

SEQ ID NO: 287 - DNA, 219 bp, rat
  misc_feature (1)..(219): Clone #209  (PLSSH)
gsssbbbnnttttttttttttttttttttttttttttttttggctttgaaacttagtttattcaaagttgtgaacaacgtcaagaacacrtttgggctacttaattactattttaaggatggsgkgcngattcatagagtcagcattgtggttttcggggggagtataaagattctgcctctactttycytttacagtaaaactgtgacaaattwattg

SEQ ID NO: 288 - DNA, 243 bp, rat
  misc_feature (1)..(243): Clone #210  (PLSSH)
acagtgtggagaggctgtcggttccttatgtgccacaggtgacagatgaagatcgcctttctagaagaaaaagtattgtggatactgtatctattcaggtggatattttatccaacagtgtgccttctgatgatgtggttagcaacactgaagaaatcacttttgaagcactgaagaaagcaattgataccaatggaatggaagaacaggaaaaggaaaagagacgtcttgtgatagagaagt

SEQ ID NO: 289 - DNA, 105 bp, rat
  misc_feature (1)..(105): Clone #222  (PLSSH)
cgtgtacgcgcgagtggaagcgcgctgctcgagtcaggtaacggtagttcgtatcgtgtatgtatcgctacttcttgcgcagacctattggatataggtcaacta

(19) United States
(12) Patent Application Publication
(10) Pub. No.: US 2004/0265804 A1
(43) Pub. Date: Dec. 30, 2004

(54) Method for identifying metastatic tumor cells

(76) Inventors: Oliver von Stein, Karlsruhe (DE); Andrea Nestl, Karlsruhe (DE); Martin Hofmann, Heidelberg (DE); Jonathan Sleemann, Bruchsal (DE); Peter Herrlich, Karlsruhe (DE)

Correspondence Address: Friedrich Kueffner, 317 Madison Avenue, Suite 910, New York, NY, 10017, US

(21) Appl. No.: 10/333,872

(22) PCT Filed: Jul. 24, 2001
(86) PCT No.: PCT/EP01/08517

(30) Foreign Application Priority Data
Jul. 25, 2000 (DE) 100 36 126.9

(51) Int. Cl. C12Q 1/68

(52) U.S. Cl. 435/6

(57) ABSTRACT

The present invention relates to a method for identifying and treating metastasizing tumor cells and to a population of cDNA sequences of metastasis-specific genes for use as tumor markers for identifying human cells and/or tissues possessing metastasis potential and for the therapy of cancer diseases.

The present invention relates to a method for identifying metastasizing tumor cells and to the identification of compounds for treating tumors.

In higher organisms, differential gene expression regulates cell growth or organogenesis in a complex manner. In particular, because of its great relevance to medicine, the elucidation of the molecular mechanisms which are involved in the development of tumor cells is the subject of intensive investigations.

However, the processes which are involved in the development of an invasive tumor from primary tumor tissue (metastasis formation) are less well investigated. It is known that some basic conditions have to be created to enable tumors to invade. These conditions relate to changes in the microenvironment of the cells, i.e. proteolysis of the cells, and to changes in adhesion properties and migration. Thus far, it has only been possible to identify a small number of genes which are expressed differentially during progression of the tumor. Some protease genes, such as uPa or matrix metalloproteases (MMP) may be mentioned by way of example (Matrisian L. M. et al., 1990, Curr. Top. Dev. Biol., 24: 219-259 and Matrisian, L. M., Bioessays, 1992, 14: 455-463). Various integrins (Riuz P. et al., 1993, Cell. Adhes. Commun., 1: 67-81), or what is termed the lymphocyte-homing receptor CD44 (Ponta et al., Frontiers in Bioscience, 1998, 3: 650-656), are known with regard to the adhesive properties.

However, the number and identity of the genes which are involved in the metastatic process are to a large extent still unknown. However, in order to obtain an improved understanding of the processes involved in tumor progression, it is necessary to clearly identify as large a number of metastasis-specific genes as possible.

In addition, there has thus far not been any suitable system available which, with justifiable expense and adequate reliability, makes it possible to draw conclusions with regard to cancer cell status and the probable existence of a tumor metastasis.

The present invention relates to a method for identifying metastasizing tumor cells, with at least one cDNA sequence, which is selected from a population in accordance with Figures A and/or B, or corresponding complete gene sequences which are derived therefrom, or functional fragments thereof, or their homologs or alleles, being used as markers for hybridizing with tumor tissue.

The present invention also encompasses the gene products (polypeptides) which are derived from the cDNA sequences which are selected from a population in accordance with Figures A and/or B or from the corresponding complete gene sequences, or functional fragments thereof or their homologs or alleles. According to the invention, the gene products also include the isoforms of these polypeptides which, while having an amino acid sequence which is altered still have the same effect as far as their function is concerned.

The invention furthermore also relates to antibodies which bind specifically to the previously mentioned gene products and which are prepared using the abovementioned cDNA sequences and/or their gene products.

According to the invention, the complete gene sequences also include, in addition to the coding nucleotide sequences, the appurtenant regulatory regions upstream and downstream of the coding regions. The regulatory regions are to be understood as being functional structures such as promoters, terminators, target sequences, retention signals, translation enhancers, splicing elements and polyadenylation signals.

According to the invention, a functional fragment is a nucleotide sequence which encodes a corresponding polypeptide which, for its part, exhibits a specific activity of the corresponding whole-length protein. In addition, a functional fragment is to be understood, according to the invention, as being a nucleotide sequence which leads, under stringent conditions, to a specific hybridization signal. In this context, the length of the functional fragment can vary in a range from at least 10 up to several hundred nucleotides. The same applies to a functional fragment of an encoded protein, the length of which fragment can vary in a range from at least 10 to 250 amino acids.

According to the invention, homologs are to be understood as being complementary nucleotide sequences or nucleotide sequences which hybridize with the cDNA sequences which are selected from a population according to Fig. A and/or B or complete gene sequences which are derived therefrom or functional gene fragments. Hybridizing sequences include similar sequences which are selected from the group of DNA or RNA which interact specifically, under stringent conditions, with the cDNA sequences according to the invention. A preferred, but not limiting, example of stringent hybridization conditions is hybridization in 6× sodium chloride/sodium citrate buffer (SSC) at 45° C. followed by one or more washing steps in 0.2×SSC, 0.1% SDS at 65° C.

Alleles are sequences which, despite having a nucleotide sequence which differs, still possess the desired functions, that is are functionally equivalent. Functional equivalents consequently include naturally occurring variants of the sequences described herein and also artificial nucleotide sequences, e.g. nucleotide sequences which are obtained by chemical synthesis and which are matched to the codon usage of the organism.

A functional equivalent (allele) is also understood, in particular, as meaning natural or artificial mutations of the originally isolated gene sequences or of the corresponding complete gene sequences, or of their homologs which encode polypeptides involved in tumor metastasis, which still exhibit the desired function. Mutations comprise substitutions, additions, deletions, exchanges or insertions of one or more nucleotide residues. Mutations can give rise both to functional changes in the regulatory regions of a gene and to a protein whose activity is altered.

Consequently, the present invention also encompasses, for example, those nucleotide sequences which are obtained by modifying the cDNA sequences according to the invention or their complete gene sequences or functional fragments thereof, or their homologs. The aim of such a modification can, for example, be that of further delimiting the coding sequence or else, for example, be that of inserting further restriction enzyme cleavage sites. Functional equivalents are also those variants in which the regulatory regions are altered resulting, for example, in gene expression being attenuated or augmented as compared with the initial gene or initial gene fragment.

According to the invention, the nucleotide sequences encoding metastasis-specific polypeptides or their isoforms can be natural, chemically synthesized, modified or artificially produced nucleotide sequences. Furthermore, the previously mentioned nucleotide sequences can consist of heterologous nucleotide sequences and are mixtures of the previously described nucleotide sequences. In addition to this, functionally equivalent sequences also include those which possess a nucleotide sequence which has been altered and which confers an altered activity, which may be attenuated or augmented, on the gene product which is encoded by it.

In addition, the invention relates to artificial nucleotide sequences as long as they, as described above, impart the desired properties. These artificial nucleotide sequences can, for example, be obtained by “back-translating” proteins which have been constructed by molecular modeling, or by means of in-vitro selection. Coding nucleotide sequences which are obtained by back-translating a polypeptide sequence in accordance with the codon usage which is specific for humans are particularly suitable. A skilled person who is familiar with recombinant methods can readily determine the specific codon usage by carrying out computer analyses of other known genes.

According to the invention, the previously mentioned cDNA sequences are isolated from defined rat adenocarcinoma systems. In a preferred embodiment of the present invention, the adenocarcinoma system is the rat tumor system 13762NF (mammary carcinoma; Neri et al., JNCI, 1982, Vol. 68 (3), 507-517). The cell line MTPa was selected as a nonmetastisizing cell line. According to the invention, the metastasizing cell line is represented by the cell line MTLY. The rat pancreas carcinoma system Bsp73, together with the cell lines 1AS or 10AS and ASML represent another system which is preferred in accordance with the invention (Matzku et al., Invasion Metastasis, 1983, (3): 109-123). The cell lines G-subline and, respectively, AT-1, AT-3, MatLu and MatLyLu, as nonmetastasizing and, respectively, metastasizing cell lines, are mentioned as an example of a rat prostate carcinoma system (Isaacs et al., Prostate, 1986, (9): 261-281). However, this choice is not limiting for the present invention; on the contrary, it also includes the use of other suitable cell lines or carcinoma systems.

A crucial advantage of using rat cell lines is that, in vivo, the metastatic cells exhibit a metastasizing behavior which is analogous to that of human cancer cells (Neri et al., Int. J. Cancer, 1981, 28: 731-738; Matzku et al., Invasion Metastasis, 1983, 3: 109-123). In addition to this, the cell lines are simpler to manipulate than human tumor tissue. Furthermore, as compared with human tumor tissue, using defined rat cell lines offers the advantage that the results are highly reproducible. In particular, in this system, there is the possibility of genetically altering cell lines and investigating, in test systems, the properties which have been acquired or lost. Furthermore, simple transfer to syngenic animals is possible whereas, because of interfering immune responses, human tumor tissue would have to be transferred to an elaborate artificial system of an immunodeficient recipient. In addition to this, some hybridizations using human mammary carcinoma cell lines were also carried out. For example, the cell lines MDA-MB231 and MDA-MB-468 (Cailleau et al., J. Natl. Cancer Inst., 1974, 661-674 and Cailleau et al., In Vitro, 1978 (14): 911-915) which have been used were employed, inter alia, in order to demonstrate, for example by means of cross-hybridizations, that the “rat sample” employed is suitable for analyzing human tumor material.

Methods which are known per se are used for identifying the large number, according to the invention, of differentially expressed transcripts of metastasis-specific genes. Mention should be made here of SSH (subtractive suppression hybridization; Diatchenko et al., Proc. Natl. Acad. Sci., 1996, 93: 6025-6030) difference analysis, which is particularly suitable for identifying rare transcripts, and of an effective afterselection method, which follows it and which is characterized by high sample throughput and a high degree of reliability and which virtually rules out selecting falsely positive or falsely negative clones (von Stein et al., Nucleic Acids Research, 1997, 25 (13): 2598-2602). Northern blot analyses and RT-PCR analyses are other methods of choice which should be mentioned for determining, in accordance with the invention, the metastasis-specific expression of selected cDNA clones, for the purpose of identifying metastasizing tumor cells, which methods do not, however, have a limiting effect on the present invention.

In this connection, a special embodiment of the method according to the invention comprises the following steps:

A) making a PCR-based subtractive cDNA gene library proceeding from total mRNA populations derived from metastasizing and nonmetastasizing tumor cells,

B) identifying a population of different cDNA clones in which each cDNA sequence represents an individual (potential) metastasis-specific gene,

C) constructing the complete gene sequence on the basis of these cDNA clones,

D) preparing a sense RNA probe and an antisense RNA probe starting with at least one sequence which is selected from this cDNA population or the corresponding complete gene sequences which are derived therefrom, in particular from a population in accordance with Figures A and/or B,

E) making suitable thin layer preparations of the tumor material to be investigated,

F) hybridizing the tumor material to be investigated in situ with the previously mentioned sense and antisense RNA probes, and

G) identifying metastasizing tumor cells by detecting positive hybridization products.

In another embodiment of the present invention, marker genes which are involved in the metastasis of tumor cells are identified by the steps of

a) making a PCR-based subtractive cDNA gene library proceeding from total mRNA populations derived from metastasizing and nonmetastasizing tumor cells,

b) identifying a population of different cDNA clones in which each cDNA sequence represents an individual (potential) metastasis-specific gene,

c) constructing the complete gene sequence on the basis of these cDNA clones,

d) cloning the cDNA and/or complete gene sequence into an expression vector both in the sense reading direction and the antisense reading direction,

e) transferring and subsequently expressing this/these vector(s) into and in nonhuman mammalian cell lines, preferably rat cell lines,

f) validating the involvement in the metastasis of tumor cells of the genes identified in step b) by indentifying invasive tumors.

In addition, the method according to the invention for identifying metastasizing tumor cells is characterized by the fact that at least one cDNA sequence selected from a population in accordance with Fig. B is involved in the metastasis of pancreas tumor tissue while at least one cDNA sequence selected from a population in accordance with Fig. A is involved in the metastasis of breast tumor tissue.

An essential feature of the present invention is the provision of an exceptionally large population of different cDNA clones which are representative of a correspondingly large number of different, i.e. individual, metastasis-specific genes such that it is possible to carry out a molecular typing of the different clinical manifestations of a cancer disease with a high degree of reliability. In the same way, it is possible to draw conclusions with regard to case control, i.e. the cancer cell stage of the different manifestations, and, in particular, with regard to the potential of the tumor tissue being investigated for metastasizing.

The present invention consequently makes available a multiplicity of potential marker sequences, marker genes and/or corresponding proteins for identifying and/or treating invasive tumor cells and/or tumor tissues or cells and/or tissues which have a high potential for metastasizing.

In another embodiment of the present invention, the method according to the invention is characterized in that the cDNA sequences in accordance with Fig. C are preferably suitable for use as markers for identifying metastasizing human breast and/or colon tumor tissue. In this connection, reference may, by way of example, be made, in particular, to the sequences which are designated CD 24 and S7 in Fig. C and which exhibited the best crosshybridization results with human tumor tissue. CD 24 is a differentiation-specific protein which appears during the maturation of B lymphocytes and T lymphocytes (Fischer G. F. et al., 1990, J. Immunol., 144: 638-641). It is possible to use CD 24 mRNA to detect metastasizing human colon tissue. The main task of the ribosomal protein S7 is that of ensuring that 16S RNA folds correctly (Wimberley, B. T. et al., 1997, Structure, 5: 1187-1198). It has not previously been known that S7 is involved in tumor progression. Consequently, the present invention is making available, in the form of S7 mRNA, a novel tumor marker for metastasizing breast cell tissue. Lymph node tissue metastases which originate from an invasive breast cell carcinoma are indicated by an increase in the expression of both these tumor markers.

Fig. D presents a comparison of the expression of genes and tumor-associated molecules in cell lines which differ in their metastatic potential. Fig. E shows the results of the in-situ hybridizations.

The unambiguous identification, according to the invention, of cDNA clones which are involved in the metastasis of tumor tissue makes it possible, in a simple manner, both to carry out an extensive cloning of the complete genes and to carry out a functional analysis of the genes which are involved, in humans, in the expression of a metastatic phenotype. At the same time, this opens up the possibility of investigating a large number of metastasis-specific regulatory sequences. The insights which are obtained will be very helpful, particularly in connection with the further elucidation of molecular mechanisms for regulating the formation of metastases by tumors.

The findings of the present invention are furthermore suitable for improving the diagnosis of cancer diseases, in particular the typing of the cancer cell stage, and, following on from that, developing more efficient options for therapy. In order to treat tumor diseases, it is possible to conceive of deriving complementary (antisense) mRNA sequences, in particular short oligonucleotides, from the previously mentioned cDNA sequences and then posttranscriptionally decreasing or preventing the expression of metastasis-specific genes by introducing these antisense mRNA sequences into tumor cells. It is furthermore possible to conceive of a tumor disease treatment in which the function of metastasis-specific polypeptides is impeded or blocked, at the posttranslational level, by substances or ligands being specifically bound to the polypeptides, thereby decreasing or preventing metastasis of the tumor.

The present invention also relates to a method for identifying compounds for treating tumors, with a metastasis-specific gene sequence, which is derived from at least one of the cDNA sequences selected from a population in accordance with Fig. A and/or B, being expressed in nonhuman mammalian cells, for example rat cell lines, chemically, biologically and/or pharmaceutically active compounds, such as proteins, peptides or low molecular weight compounds, being incubated with the previously mentioned cells, and those compounds which exhibit a modulating effect on the expression of the genes which are associated with the tumor metastasis, or on the activity of the proteins which are encoded by the genes, being subsequently selected. It is then possible to conceive of using these compounds to produce compositions which can be used for treating tumors.

According to the invention, a modulating effect is to be understood as meaning an augmentation or an attenuation. According to the invention, this encompasses an interactive inhibition within the cells as well as regulatory interaction on the cell surface.

The metastasis-specific gene sequences according to the invention can, for example, be introduced into, and expressed in, the nonhuman mammalian cells using a suitable recombinant vector which contains at least one gene sequence according to the invention which is derived from a cDNA sequence selected from a population in accordance with Fig. A and/or B, or complete gene sequences or functional fragments thereof, for example coding sequences, and, in addition, regulatory nucleotide sequences, in particular from the group comprising promoters, terminators, target sequences, retention signals and translation enhancers, splicing elements, polyadenylation signals or resistance-mediating nucleotide sequences and nucleotide sequences for replication in the relevant target cell or integration into its genome.

The present invention further relates to a measuring system for identifying compounds for treating tumors, which system contains at least one gene which is associated with tumor metastasis and which is obtained using a cDNA sequence in accordance with Fig. A and/or B, or alleles or homologs thereof, and at least one chemically, biologically and/or pharmaceutically active compound.

This measuring system according to the invention provides the possibility of carrying extensive regulatory studies and binding studies with the primary aim of establishing whether and, where appropriate, in which ranges, the compound exerts an effect on expression of the gene, i.e. attenuates or augments this expression. The measuring system which is employed in accordance with the invention can, for example, be a nonhuman mammalian cell, preferably a defined rat cell line.

Another variant of the present invention relates to a measuring system which contains at least one tumor metastasis-related protein, or isoforms thereof, which is/are encoded by a gene sequence, or its homologs, which is/are obtained from a cDNA sequence in accordance with Fig. A and/or B, and also at least one chemically, biologically and/or pharmaceutically active compound. In this case, too, the measuring system according to the invention provides the possibility of carrying out extensive regulatory studies and binding studies with the primary aim of establishing whether and, where appropriate, in what ranges, the compound binds to a tumor metastasis-associated protein and alters, i.e. attenuates or augments, the activity of this protein.

The present invention furthermore relates to the compounds themselves and/or their use for producing compositions which are intended for treating tumor diseases and which comprise compounds which have been identified by a previously described method or using a previously described measuring system.

In addition, the present invention relates to cDNA sequences which are selected from a population in accordance with Fig. A and/or B or complete gene sequences which are derived therefrom, or their homologs, or functionally equivalent sequences, for use in one of the previously described methods or measuring systems. This also includes the use of mixtures of the cDNA sequences according to the invention. The present invention also relates to the gene products (polypeptides) which can be prepared, or derived therefrom, using the cDNA sequences according to the invention.

In the same way, the present invention relates to genetically altered nonhuman mammalian cells which contain at least one cDNA sequence according to the invention which is selected from a population in accordance with Fig. A and/or B, or complete gene sequences which are derived therefrom, or functional fragments, or the homologs or alleles, or a recombinant vector of the previously described type, or gene products of the abovementioned type.

The present invention furthermore encompasses antibodies which bind specifically to said gene products according to the invention, with it being possible to prepare these antibodies using the cDNA sequences and/or gene products according to the invention.

The invention furthermore uses a probe for specifically hybridizing with tumor tissue, with this probe being prepared from a cDNA sequence which is selected from a population in accordance with Fig. A and/or B, or from complete gene sequences which are derived therefrom, or their homologs or functionally equivalent sequences, and with the probe containing a label which is suitable for detection purposes and which is preferably not radioactive, and/or the probe being 30 nucleotides, preferably 15-20 nucleotides, particularly preferably 10 nucleotides, in length.

The present invention furthermore relates to a test kit which contains at least one cDNA sequence which is selected from a population accordance with Fig. A and/or B, or complete gene sequences which are derived therefrom, or functional gene fragments thereof, or their homologs or alleles, an instruction for preparing a previously described probe and also instructions for hybridizing and detecting nucleotide sequences in tumor tissue. Preference is given to using a mixture of several relevant cDNA sequences.

The present invention furthermore relates to the use of compounds, which have been identified by a method according to the invention and/or using a measuring system according to the invention, for producing compositions which can be used for treating tumors. In the same way, the present invention encompasses the use of the antibodies according to the invention for producing compositions which can be used for treating tumors. In addition, the cDNA sequences according to the invention, gene products, antibodies and/or parts thereof, and/or the compounds according to the invention which are involved in the development of tumor metastases, are suitable for being used in areas of tumor metastasis diagnosis and/or tumor metastasis therapy. In areas of tumor diagnosis, for example, this also includes identifying further tumor metastasis markers of human origin.

The present invention is characterized in more detail by the following implementation examples which do not, however, have a limiting effect on the invention:

General Methods

The isolation and analysis of nucleic acids, general cloning techniques, DNA sequencing, RNA techniques, and methods for transferring and hybridizing nucleic acids, are described in Sambrook et al., Molecular cloning: A laboratory manual, 1989, Cold Spring Harbour Laboratory Press, as are general procedures for culturing cells and histological techniques.

Subtractive Cloning of Differentially Expressed Genes (SSH)

The preparation of a differential cDNA library (subtraction library) in accordance with the principle of what is termed suppressive subtractive hybridization is described in Diatchenko L. et al., 1996, Proc Natl Acad Sci USA, 93: 6025-6030. The subtraction reaction was effected using the CLONTECH PCR-Select cDNA Subtraction kit from Clontech Laboratories, Palo Alto, USA, in accordance with the manufacturer's instructions.

Analysis of the cDNA Clones of the Subtraction Library, Taking as an Example the Rat Cell Line 13762

In order to use a southern blot analysis to test the efficiency of the subtraction, it is necessary to prepare amplified RsaI fragments of the driver, MTPa, since the subtracted library, ML, also consists of RsaI-digested cDNA fragments. After the MTPa cDNA had been digested with RsaI, adaptors (Eco linkers) were ligated to the fragments, which were then amplified using the corresponding PCR primers (Eco linker primers) in accordance with the conditions for the 2nd SSH PCR. MTLY was used in the form of the 1c tester fraction (see SSH manual).

Ligating into a TA Vector

The PCR products from the 2nd SSH PCR round were ligated into a TA vector (pCRII.1, Invitrogen). Cloning an insert into the pCR.II.1 vector interrupts the coding sequence of the β-galactosidase gene, which means that it is possible to distinguish recombinant clones (white) from empty vectors (blue). However, since the Taq polymerase which is used possesses proofreading activity, it is necessary to incubate the PCR products in a further step, after a preceding phenol/chloroform extraction, with DATP and another Taq DNA polymerase (Eurobio Taq polymerase) at 72° C. for 15 min and then perform another phenol chloroform extraction. The ligation was effected using the T4 DNA ligase (Invitrogen), which is enclosed with the vector, in a ratio of 1:1 (for each 25 ng of vector and subtracted library).

Transforming into Electrocompetent Bacteria and Blue/White Sorting

The library which was ligated in the pCR11.1 vector was transformed into electrocompetent bacteria (ELEKTROMAX, strain DH10B, Invitrogen) and the bacteria were plated out on 15 cm bacterial dishes. In addition to LB agar, the dishes contained the selection antibiotic ampicillin (100 μg/ml) and also 100 μM IPTG and X-Gal (50 μg/ml) for blue/white sorting. The bacterial dishes were incubated at 37° C. until small colonies were visible. In order to make it easier to distinguish between white and blue colonies, the plates were subsequently incubated at 4° C.

Picking Clones and Colony PCR

A total of 1985 clones were picked by the blue/white selection procedure and transferred to sterile 96-well microtiter plates which contained LB medium and ampicillin (100 μg/μl). The bacteria were allowed to grow in these plates at RT for 14 hours while being shaken gently. In order to implement the colony PCR, 10 μl of each bacterial culture were removed, using a multichannel pipette, and transferred to a 96-well PCR plate and mixed, on this latter plate, with in each case 90 μl of sterile water. For denaturing, the plate was incubated at 95° C. for 5 min. in the PCR machine (Perkin-Elmer 9600 thermal cycler). A multichannel pipette was then used to transfer 5 μl of each lysate into a second 96-well PCR plate into which in each case 90 μl of the PCR mixture had been introduced. The cDNA fragments were amplified using the subtraction nested primers 1 and 2.

PCR Mixture (50 μl Assay):

0.5 U of Taq polymerase (Promega), 5 μl of 10×PCR buffer (Promega), 200 μM dNTPs, in each case 10 μmol of the nested primers 1 and 2 of the subtraction reaction+5 μl of the bacterial lysate

PCR Conditions:

94° C., 30 seconds −68° C., 30 seconds −72° C., 30 seconds

Reverse Northern Blot Analysis

A reverse northern blot analysis was used to test which clone did indeed contain differentially expressed cDNA fragments. For this, the amplified cDNA fragments (colony PCR) were loaded in identical order onto 2 high-density agarose gels (Centipede™ gel electrophoresis chambers, Owl Scientific, Woburn, USA). 2×96 samples (2 microtiter plates) were loaded per gel. In this connection, it is of the greatest importance that both the gels are loaded exactly equally. In each case, a GAPDH control was loaded onto the last position for the purpose of subsequently quantifying the signal strength. After alkaline blotting (see southern blot analysis), the membrane duplicates were hybridized with ³²P-labeled tester (MTLY) cDNA or driver (MTPa) cDNA (in each case digested with RsaI) and analyzed by autoradiography.

Preparing Hybridization Probes from the Isolated cDNA Fragments

In order to prepare the probes, the cDNA fragments were amplified from the corresponding bacterial clones, following the principle of colony PCR, using the subtraction nested primers 1 and 2.

PCR Mixture (50 μl Assay):

0.5 U of Taq polymerase (Promega), 5 μl of 10×PCR buffer (Promega), 200 μM dNTPs, in each case 10 μmol of the nested primers 1 and 2 of the subtraction reaction+5 μl of the bacterial lysate

PCR Conditions:

94° C., 30 seconds −68° C., 30 seconds −72° C., 30 seconds

The cDNA fragments which were obtained, and which were differentially expressed, are collected in Fig. A and Fig. B, with Fig. A representing cDNA fragments obtained from a mammary carcinoma-specific library (MLSSH) and Fig. B representing cDNA fragments obtained from a pancreas-specific library (PLSSH).

### DESCRIPTION OF THE FIGURES

Fig. A:

List of the cDNA sequences contained in the mammary carcinoma-specific library which were obtained by suppressive substractive hybridization (MLSSH).

Fig. B:

List of the cDNA sequences contained in the pancreas-specific library which were obtained by suppressive substractive hybridization (PLSSH).

Fig. C:

cDNA sequences of CD 24 and S7, as tumor markers for invasive human breast tumor tissue and/or colon tumor tissue.

Fig. D:

Comparison of the expression of novel genes and tumor-associated molecules in cell lines having different metastatic potential. The figure consists of northern blots from four different rat tumor systems and two human breast cancer cell lines. Apart from the MN081/MT450 couple, all the tumor systems employed consist of clonal cell lines which are derived from a primary tumor and which only differ in their capacity for metastasis. This metastatic potential is indicated. Each lane in the northern blots was loaded with in each case 2 μg of poly-A⁺ RNA. The blots were hybridized with various differentially expressed cDNAs which were isolated in the MLSSH and PLSSH subtraction libraries (screens). The hybridization probes which were used were selected randomly from the metastasis-specific genes which were isolated in the MLSSH and PLSSH screen. In each case, they represent 10% of the isolated genes. A rough classification (correlated index) is intended to describe the correlation between the expression of the genes and the metastatic potential of the cell lines being investigated. Genes whose expression is upregulated in all the metastatic cell lines and is not present, or is significantly reduced, in all the nonmetastatic cells were indicated by +++. Genes which are clearly expressed differentially, but not exclusively, in metastatic cells were indicated by ++ or + depending on the strength with which expression was correlated with the metastatic potential. Expression of some clones exhibited a strong association with metastatic potential in only one tumor progression model.

Fig. E:

In-situ hybridization. Sections (6 μm) of formaldehyde-fixed and paraffin-embedded human carcinomas were hybridized with ³⁵S-UTP or fluorescence-labeled sense and antisense RNA and subsequently counterstained with haematoxylin and eosin (H and E). The carcinomas were graded in accordance with the UICC guidelines. The scales represent 100 μm (a, b, e and f), 40 μm (c and d) and 20 μm (g and h).

a-b (a, sense control; b, radioactively labeled antisense CD24 probe): CD24 expression is a moderately differentiated adenocarcinoma of the intestine, graded as pT3C; p21; pN0; pM1. The section shows significant overexpression of CD24 in intraductal carcinoma cells (located in the cytoplasm) whereas the normeoplastic mucosa is only weakly positive (constant basal expression).

c-d (c, sense control; d, radioactively labeled antisense CD24 probe): CD24 expression in a moderately invasive ductal, moderately differentiated breast carcinoma, graded as pT2; pN1bii. It was possible to detect a positive signal in the carcinoma but not in the surrounding stroma.

e-f (e, sense control; f, radioactively labeled antisense S7 probe): S7 expression in a moderately differentiated adenocarcinoma of the intestine, graded as pT3C; p21; pN0; pM1. The section shows significant overexpression of S7 (predominantly located in the nucleus) in the carcinoma, but only very weak expression in the normeoplastic mucosa.

h-g (h, sense control; 9, fluorescence-labeled antisense S7 probe): expression of S7 in an invasive ductal breast carcinoma which is not differentiated to any extent and which is graded PT-1C; G3, N-1B1 or I, R—O, L-1. While it was possible to detect a strong nuclear signal in the intraductal carcinoma, hardly any signal was observed in the stroma.

1. A method for identifying metastasizing tumor cells, characterized in that at least one cDNA sequence which is selected from a population in accordance with Fig. A and/or B, or complete gene sequences which are derived therefrom, or functional gene fragments thereof, or their homologs or alleles, is/are used for hybridizing with tumor tissue.

2. The method as claimed in claim 1, characterized in that the cDNA sequences which are selected from a population in accordance with Fig. B are involved in the metastasis of pancreas tumor tissue and/or the cDNA sequences which are selected from a population in accordance with Fig. A are involved in the metastasis of breast tumor tissue.

3. The method as claimed in claim 1 or 2, characterized in that the cDNA sequences in accordance with Fig. C are involved in the metastasis of human breast tumor tissue and/or colon tumor tissue.

4. A method for identifying compounds for treating tumors, characterized in that
a) a metastasis-specific gene sequence which is derived from at least one of the cDNA sequences which are selected from a population in accordance with Fig. A and/or B is expressed in nonhuman mammalian cells,
b) chemically, biologically and/or pharmaceutically active compounds are incubated with the abovementioned cells,
c) those compounds which exhibit a modulating effect on the expression of the genes associated with the tumor metastasis, or on the activity of the proteins which are encoded by the genes, are selected.

5. A measuring system for identifying compounds for treating a tumor, which comprises at least one gene, which is associated with the tumor metastasis and which is obtained from a cDNA sequence in accordance with Fig. A and/or B, and also at least one chemically, biologically and/or pharmaceutically active compound.

6. A measuring system as claimed in claim 5, which comprises at least one protein, which is associated with the tumor metastasis and which is encoded by a gene which is obtained from a cDNA sequence in accordance with Fig. A and/or B, and also at least one chemically, biologically and/or pharmaceutically active compound.

7. A compound which is identified by a method as claimed in claim 4 or using a measuring system as claimed in claim 5 or 6.

8. A cDNA sequence which is selected from a population in accordance with Fig. A and/or B, or a complete gene sequence which is derived therefrom, or a functional gene fragment, or its homolog of allele, for use in a method as claimed in one of claims 1 to 4 or in a measuring system as claimed in claim 5 or 6.

9. A gene product which is prepared using at least one cDNA sequence as claimed in claim 8.

10. A genetically altered nonhuman mammalian cell which contains at least one cDNA sequence as claimed in claim 8 or at least one gene product as claimed in claim 9.

11. An antibody for specifically binding to a gene product as claimed in claim 9, which is prepared using at least one cDNA sequence as claimed in claim 8 or one gene product as claimed in claim 9.

12. A probe for specifically hybridizing with tumor tissue, characterized in that it is prepared from at least one cDNA sequence as claimed in claim 8, it contains a label which is suitable for detection purposes, and/or it is 30 nucleotides, preferably 15-20 nucleotides, particularly preferably 10 nucleotides, in length.

13. A test kit which comprises at least one cDNA sequence as claimed in claim 8, an instruction for preparing a probe as claimed in claim 12 and instructions for hybridizing and detecting nucleotide sequences in tumor tissue.

14. The use of compounds as claimed in claim 7 for producing compositions for treating cancer diseases.

15. The use of antibodies as claimed in claim 11 for producing compositions for treating cancer diseases.
